(19) United States
(12) Patent Application Publication
(10) Pub. No.: US 2006/0084086 A1
(43) Pub. Date: Apr. 20, 2006

(54) Proteins, polynucleotides encoding them and methods of using the same

(76) Inventors: Kimberly A. Spytek, Ellington, CT (US); Corine A.M. Vernet, Chernex (CH); Xiaojia (Sasha) Guo, Woodbridge, CT (US); Raymond J. Taupier JR., East Haven, CT (US)

Correspondence Address: CURAGEN CORPORATION, 322 EAST MAIN STREET, BRANFORD, CT, 06405, US

(21) Appl. No.: 11/156,300

(22) Filed: Jun. 16, 2005

Related U.S. Application Data
(63) Continuation-in-part of application No. 10/080,334, filed on Feb. 21, 2002.
(60) Provisional application No. 60/270,523, filed on Feb. 21, 2001.
(60) Provisional application No. 60/322,712, filed on Sep. 17, 2001.
(60) Provisional application No. 60/311,980, filed on Aug. 13, 2001.
(60) Provisional application No. 60/330,307, filed on Oct. 18, 2001.
(60) Provisional application No. 60/278,796, filed on Mar. 26, 2001.
(60) Provisional application No. 60/281,521, filed on Apr. 4, 2001.
(60) Provisional application No. 60/276,677, filed on Mar. 16, 2001.
(60) Provisional application No. 60/311,595, filed on Aug. 10, 2001.
(60) Provisional application No. 60/270,220, filed on Feb. 21, 2001.
(60) Provisional application No. 60/274,295, filed on Mar. 8, 2001.
(60) Provisional application No. 60/318,526, filed on Sep. 10, 2001.
(60) Provisional application No. 60/286,548, filed on Apr. 25, 2001.
(60) Provisional application No. 60/291,765, filed on May 17, 2001.
(60) Provisional application No. 60/270,797, filed on Feb. 23, 2001.
(60) Provisional application No. 60/276,400, filed on Mar. 16, 2001.
(60) Provisional application No. 60/270,810, filed on Feb. 23, 2001.

(51) Int. Cl. C12Q 1/68; C07H 21/04; C12P 21/06; C12N 9/02

(52) U.S. Cl. 435/6; 435/69.1; 435/189; 435/320.1; 435/325; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. patent application Ser. No. 10/080,334, filed Feb. 21, 2002, which claims priority to U.S. Ser. No. 60/270,523, filed Feb. 21, 2001; U.S. Ser. No. 60/322,712, filed Sep. 17, 2001; U.S. Ser. No. 60/311,980, filed Aug. 13, 2001; U.S. Ser. No. 60/330,307, filed Oct. 18, 2001; U.S. Ser. No. 60/278,796, filed Mar. 26, 2001; U.S. Ser. No. 60/281,521, filed Apr. 4, 2001; U.S. Ser. No. 60/276,677, filed Mar. 16, 2001; U.S. Ser. No. 60/311,595, filed Aug. 10, 2001, U.S. Ser. No. 60/270,220, filed Feb. 21, 2001; U.S. Ser. No. 60/274,295, filed Mar. 8, 2001; U.S. Ser. No. 60/318,526, filed Sep. 10, 2001; U.S. Ser. No. 60/286,548, filed Apr. 25, 2001; U.S. Ser. No. 60/291,765, filed May 17, 2001; U.S. Ser. No. 60/270,797, filed Feb. 23, 2001; U.S. Ser. No. 60/276,400, filed Mar. 16, 2001; and U.S. Ser. No. 60/270,810, filed Feb. 23, 2001, each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: Androgen-Regulated Short-Chain Dehydrogenase/Reductase-like, Insulysin-like, BCSC-1-like, Lymphocyte Antigen Precursor-like, Lymphocyte Antigen LY-6F-like, TASK 4-like, Copper Transporter-like, Cytokeratin-like, Protocadherin-like, Protocadherin Beta-like, and Keratin 18-like. More particularly, the invention relates to nucleic acids encoding novel polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOs:1, 3, 5, 7, 9, 11, 13, 15, 17, 19, 21, 23, 25, 27, 29, and 31. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOs:2, 4, 6, 8, 10, 12, 14, 16, 18, 20, 22, 24, 26, 28, 30, and 32. The nucleic acid can be, for example, a genomic DNA fragment or a cDNA molecule that includes the nucleic acid sequence of any of SEQ ID NOs:1, 3, 5, 7, 9, 11, 13, 15, 17, 19, 21, 23, 25, 27, 29, and 31.

Also included in the invention is an oligonucleotide, e.g., an oligonucleotide which includes at least 6 contiguous nucleotides of a NOVX nucleic acid (e.g., SEQ ID NOs:1, 3, 5, 7, 9, 11, 13, 15, 17, 19, 21, 23, 25, 27, 29, and 31) or a complement of said oligonucleotide. Also included in the invention are substantially purified NOVX polypeptides (SEQ ID NOs:2, 4, 6, 8, 10, 12, 14, 16, 18, 20, 22, 24, 26, 28, 30, and 32). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., trauma, regeneration (in vitro and in vivo), viral/bacterial/parasitic infections, Von Hippel-Lindau (VHL) syndrome, Alzheimer's disease, stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, behavioral disorders, addiction, anxiety, pain, actinic keratosis, acne, hair growth diseases, allopecia, pigmentation disorders, endocrine disorders, connective tissue disorders, such as severe neonatal Marfan syndrome, dominant ectopia lentis, familial ascending aortic aneurysm, isolated skeletal features of Marfan syndrome, Shprintzen-Goldberg syndrome, genodermatoses, contractural arachnodactyly, inflammatory disorders such as osteo- and rheumatoid-arthritis, inflammatory bowel disease, Crohn's disease; immunological disorders, AIDS; cancers including but not limited to lung cancer, colon cancer, Neoplasm; adenocarcinoma; lymphoma; prostate cancer; uterus cancer, leukemia or pancreatic cancer; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, Neurologic diseases, Brain and/or autoimmune disorders like encephalomyelitis, neurodegenerative disorders, immune disorders, hematopoietic disorders, muscle disorders, inflammation and wound repair, bacterial, fungal, protozoal and viral infections (particularly infections caused by HIV-1 or HIV-2), pain, acute heart failure, hypotension, hypertension, urinary retention, osteoporosis, Treatment of Albright Hereditary Ostoeodystrophy, angina pectoris, myocardial infarction, ulcers, benign prostatic hypertrophy, arthrogryposis multiplex congenita, osteogenesis imperfecta, keratoconus, scoliosis, duodenal atresia, esophageal atresia, intestinal malrotation, Pancreatitis, Obesity Systemic lupus erythematosus, Autoimmune disease, Emphysema, Scleroderma, allergy, ARDS, Neuroprotection, Fertility Myasthenia gravis, Diabetes, obesity, Growth and reproductive disorders Hemophilia, Hypercoagulation, Idiopathic thrombocytopenic purpura, Immunodeficiencies, Graft vesus host, Adrenoleukodystrophy, Congenital Adrenal Hyperplasia, Endometriosis, Xerostomia, Ulcers, Cirrhosis, Transplantation, Diverticular disease, Hirschsprung's disease, Appendicitis, Arthritis, Ankylosing spondylitis, Tendinitis, Renal artery stenosis, Interstitial nephritis, Glomerulonephritis, Polycystic kidney disease, erythematosus, Renal tubular acidosis, IgA nephropathy, anorexia, bulimia, psychotic disorders, including anxiety, schizophrenia, manic depression, delirium, dementia, severe mental retardation and dyskinesias, such as Huntington's disease and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed herein and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, and antibodies that selectively bind to such polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1 | CG57413-01 | 1 | 2 | Androgen-Regulated Short-Chain |
| | | | | Dehydrogenase/Reductase-like |
| 2a | CG57433-01 | 3 | 4 | Insulysin-like |
| 2b | CG57433-02 | 5 | 6 | Insulysin-like |
| 3 | CG57362-01 | 7 | 8 | BCSC-1 like |
| 4a | CG56417-01 | 9 | 10 | Lymphocyte Antigen Precursor-like |
| 4b | CG56417-02 | 11 | 12 | Lymphocyte Antigen Precursor-like |
| 4c | CG56417-03 | 13 | 14 | Lymphocyte Antigen Precursor-like |
| 4d | CG56417-04 | 15 | 16 | Lymphocyte Antigen LY-6F-like |
| 4e | 172885384 | 17 | 18 | Lymphocyte Antigen LY-6F-like |
| 5 | CG57220-04 | 19 | 20 | TASK 4-like |
| 6 | CG57458-01 | 21 | 22 | Copper Transporter-like |
| 7 | CG57454-01 | 23 | 24 | Cytokeratin-like |
| 8a | CG57446-01 | 25 | 26 | Protocadherin-like |
| 8b | CG57444-01 | 27 | 28 | Protocadherin-like |
| 8c | CG57442-01 | 29 | 30 | Protocadherin Beta-like |
| 9 | CG57484-01 | 31 | 32 | Keratin 18-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A disclosed NOV1 nucleic acid of 872 nucleotides (also referred to as CG57413-01) encoding a novel Androgen-Regulated Short-Chain Dehydrogenase/Reductase-like protein is shown in Table 1A. An open reading frame was identified beginning with an ATG initiation codon at nucleotides 11-13 and ending with a TAA codon at nucleotides 845-847. Putative untranslated regions, if any, upstream from the initiation codon and downstream from the termination codon are underlined in Table 1A, and the start and stop codons are in bold letters.

TABLE 1A
NOV1 Nucleotide Sequence (SEQ ID NO:1).

CTCGTGAAGGATGGTACGTGATGCTCTTGTTTCCCTTGCCGATAGGAAGT
TCTTTGCTGGTGGAGTGTGTAGAACAAATGTGCAGCTTCCTGGCAAGGTA
GTGGTGATCACTGGCGCCAACACGGGCATTGGCAAGGAGACGGCCAGAGA
GCTCGCTAGCCGAGGAGCCCGAGTCTATATTGCCTGCAGAGATGTACTGA
AGGGGGAGTCTGCTGCCAGTGAAATCCGAGTGGATACAAAGAACTCCCAG
GTGCTGGTGCGGAAATTGGACCTATCCGACACCAAATCTATCCGAGCCTT
TGCTGAGGGCTTTCTGGCAGAGGAAAAGCAGCTCCATATTCTGATCAACA
ATGCGGGAGTAATGATGTGTCCATATTCCAAGACAGCTGATGGCTTTGAA
ACCCACCTGGGAGTCAACCACCTGGGCCACTTCCTCCTCACCTACCTGCT
CCTGGAGCGGCTAAAGGTGTCTGCCCCTGCACGGGTGGTTAATGTGTCCT
CGGTGGCTCACCACATTGGCAAGATTCCCTTCCACGACCTCCAGAGCGAG
AAGCGCTACAGCAGGGGTTTTGCCTATTGCCACAGCAAGCTGGCCAATGT
GCTTTTTACTCGTGAGCTGGCCAAGAGGCTCCAAGGCACCGGGGTCACCA
CCTACGCAGTGCACCCAGGCGTCGTCCGCTCTGAGCTGGTCCGGCACTCC
TCCCTGCTCTGCCTGCTCTGGCGGCTCTTCTCCCCCTTTGTCAAGACGGC
ACGGGAGGGGGCGCAGACCAGCCTGCACTGCGCCCTGGCTGAGGGCCTGG
AGCCCCTGAGTGGCAAGTACTTCGGTGTCTCCAAGGGCCCGAAATAACAA
AACAGCTGAGCGCCTATGGAAT

The NOV1 nucleic acid sequence maps to chromosome 14 and has 584 of 797 bases (73%) identical to a gb:GENBANK-ID:AX041971|acc:AX041971.1 mRNA from Homo sapiens (Sequence 1 from Patent WO0065067) (E=2.1 e⁻⁸⁴). Similiarity information was assessed using public nucleotide databases including all GenBank databases and the GeneSeq patent database. Chromosome information was assigned using OMIM and the electronic northern tool from Curatools to derive the the chromosomal mapping of the SeqCalling assemblies, Genomic clones, and/or EST sequences that were included in the invention.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., mRNA from Homo sapiens (Sequence 1 from Patent WO0065067), matched the Query NOV1 sequence purely by chance is 2.1e⁻⁸⁴. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., ncbi website. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, 1996.

The disclosed NOV1 polypeptide (SEQ ID NO:2) encoded by SEQ ID NO:1 has 278 amino acid residues and is presented in Table 1B using the one-letter amino acid code. Signal P, Psort and/or Hydropathy results predict that NOV1 does not contain a signal peptide and is likely to be localized to the mitochondrial matrix space with a certainty of 0.4843 and to the microbody (peroxisome) with a certainty of 0.3713.

TABLE 1B
Encoded NOV1 protein sequence (SEQ ID NO:2).

MVRDALVSLADRKFFAGGVCRTNVQLPGKVVVITGANTGIGKETARELAS
RGARVYIACRDVLKGESAASEIRVDTKNSQVLVRKLDLSDTKSIRAFAEG
FLAEEKQLHILINNAGVMMCPYSKTADGFETHLGVNHLGHFLLTYLLLER
LKVSAPARVVNVSSVAHHIGKIPFHDLQSEKRYSRGFAYCHSKLANVLFT
RELAKRLQGTGVTTYAVHPGVVRSELVRHSSLLCLLWRLFSPFVKTAREG
AQTSLHCALAEGLEPLSGKYFGVSKGPK

The NOV1 amino acid sequence has 197 of 260 amino acid residues (75%) identical to, and 228 of 260 amino acid residues (87%) similar to, the 318 amino acid residue ptnr:SPTREMBL-ACC:Q9NRW0 protein from Homo sapiens (Androgen-Regulated Short-Chain Dehydrogenase/Reductase 1) (E=4.6e⁻¹⁰⁵).

NOV1 is expressed in at least the following tissues: kidney, pancreas, retina and vulva. This information was derived by determining the tissue sources of the sequences that were included in the invention including but not limited to SeqCalling sources, public EST sources, and/or RACE sources.

Possible small nucleotide polymorphisms (SNPs) found for NOV1 are listed in Table 1C. Depth, when shown, represents the number of clones covering the region of the SNP. The putative allele frequence (PAF), when shown, is the fraction of these clones containing the SNP. A dash, when shown, means that a base is not present. The sign “>” means “is changed to.” Silent, when indicated, means that the SNP did not result in an amino acid change.

TABLE 1C
SNPs

| | | | Amino | |
|---|---|---|---|---|
| | Nucleotide | Base | Acid | Base |
| Variant | Position | Change | Position | Change |
| 13375950 | 327 | A > G | 106 | Lys > Arg |

NOV1 has homology to the amino acid sequences shown in the BLASTP data listed in Table 1D.

TABLE 1D
BLAST results for NOV1

| Gene Index/ | | Length | Identity | Positives | |
|---|---|---|---|---|---|
| Identifier | Protein/Organism | (aa) | (%) | (%) | Expect |
| gi\|12861668\|dbj\|BAB32258.1\| | data source: SPTR, | 316 | 217/264 | 227/264 | e−117 |
| (AK020927) | source key: Q9NRW0, | | (82%) | (85%) | |
| | evidence: ISS˜homolog | | | | |
| | to ANDROGEN- | | | | |
| | REGULATED SHORT- | | | | |
| | CHAIN | | | | |
| | DEHYDROGENASE/REDUCTASE | | | | |
| | 1˜putative | | | | |
| | [Mus musculus] | | | | |
| gi\|16740649\|gb\|AAH16204.1\| | Similar to RIKEN | 304 | 207/264 | 216/264 | e−109 |
| AAH16204 | cDNA A930033N07 | | (78%) | (81%) | |
| (BC016204) | gene [Mus | | | | |
| | musculus] | | | | |
| gi\|9622124\|gb\|AAF89632.1\| | androgen-regulated | 318 | 187/260 | 217/260 | e−105 |
| AF167438_1 | short-chain | | (71%) | (82%) | |
| (AF167438) | dehydrogenase/reductase | | | | |
| | 1 [Homo | | | | |
| | sapiens] | | | | |
| gi\|7705791\|ref\|NP_057110.1\| | CGI-82 protein; | 318 | 187/260 | 217/260 | e−105 |
| (NM_016026) | likely ortholog of | | (71%) | (82%) | |
| | mouse cell line | | | | |
| | MC/9.IL4 derived | | | | |
| | transcript 1 [Homo | | | | |
| | sapiens] | | | | |
| gi\|12835589\|dbj\|BAB23296.1\| | cell line MC/9.IL4 | 316 | 184/266 | 218/266 | e−103 |
| (AK004413) | derived transcript | | (69%) | (81%) | |
| | 1˜data source: MGD, | | | | |
| | source key: MGI: | | | | |
| | 102581, evidence: | | | | |
| | ISS˜putative [Mus | | | | |
| | musculus] | | | | |

The homology between these and other sequences is determined by rountine techniques known in the art (e.g., ClustalW analysis). The highly conserved sequence regions may be required to preserve structural or functional properties, whereas less conserved sequence regions can potentially be altered to a much broader extent without altering protein structure or function.

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1, as disclosed in Table 1F, were collected from the Conserved Domain Database (CDD) with Reverse Position Specific BLAST analyses. This BLAST analysis software samples domains found in the Smart and Pfam collections. For Table 1F and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Table 1F lists the domain description from DOMAIN analysis results against NOV1. This indicates that the NOV1 sequence has properties similar to those of other proteins known to contain these domains.

TABLE 1F

Domain Analysis of NOV1
gnl|Pfam|pfam00106, adh_short, short chain dehydrogenase. This family
contains a wide variety of dehydrogenases. (SEQ ID NO:66)
CD-Length = 249 residues, 99.6% aligned
Score = 124 bits (310), Expect = 9e−30

| | |
|---|---|
| Query: 27 | PGKVVVITGANTGIGKETARELASRGARVYIACRDVLKGESAASEIRVDTKNSQVLVRKL 86 |
| | \|\|\| ++\|\|\|++\|\|\| \|+ \|\| \|\|+\| + \| \| \|+\|\| + \| +\| |
| Sbjct: 1 | TGKVALVTGASSGIGLAIAKRLAEEGAKVVVVDRREEKAEAAAELKA--ELGDPALFIQL 58 |
| Query: 87 | DLSDTKSIRAFAEGFLAEEKQLHILINNAGVM--MCPYSKTADGFETHLGVNHLGHFLLT 144 |
| | \|++\| +\|\|+\| + \| +\| +\|+\|\|\|\|++ \|+ + \| +\| +\|\| \| \|\|\|\| |
| Sbjct: 59 | DVTDEESIKAAVAQAVEELGRLDVLVNNAGILGPGEPFELSEDDWERVIDVNLTGVFLLT 118 |
| Query: 145 | YLLLERLKVSAPARVVNVSSVAHHIGKIPFHDLQSEKRYSRGFAYCHSKLANVLFTRELA 204 |
| | +\| + + \|+\|\|+\|\|\|\| + \|\| \|\| \|\| \| \| \|\|\| \|\| |
| Sbjct: 119 | QAVLPHMLKRSGGRIVNISSVAGLVP------------SPGLSAYSASKAAVVGFTRSLA 166 |
| Query: 205 | KRLQGTGVTTYAVHPGVVRSELV-RHSSLLCLLWRLFSPFVK---------TAREGAQTS 254 |
| | \| \|+ \|+ \|\| \| \| +++ \|+ \| \| \| \| |
| Sbjct: 167 | LELAPHGIRVNAIAPGGVDTDMTKALRSIAPADPELVERITSALVPLGRYGTPEEVANAV 226 |
| Query: 255 | LHCALAEGLEPLSGKYFGVSKG 276 |
| | \| \| ++\|+ \| \| |
| Sbjct: 227 | LFLASDOASYSVTGQTLNVDGG 248 |

Most mammalian hydroxysteroid dehydrogenases known thus far belong to the protein superfamilies of short-chain dehydrogenases/reductases (SDR) and aldo-keto reductases (AKR). Whereas members of the AKR family are soluble, cytoplasmic enzymes, SDR-type hydroxysteroid dehydrogenases are also located to other subcellular compartments, i.e. endoplasmic reticulum, mitochondria or peroxisomes. Differential localization might play an important role in influencing the reaction direction of hydroxy dehydrogenase/oxo reductase pathways by determining the available nucleotide cofactor pool. Targeting signals for different subcellular organelles in human hydroxysteroid dehydrogenases have been identified, however, in several enzymes localization signals remain to be determined. The short-chain dehydrogenases/reductases family (SDR) is a very large family of enzymes, most of which are known to be NAD- or NADP-dependent oxidoreductases. As the first member of this family to be characterized was Drosophila alcohol dehydrogenase, this family used to be called ‘insect-type’, or ‘short-chain’ alcohol dehydrogenases. Most members of this family are proteins of about 250 to 300 amino acid residues. Most dehydrogenases possess at least 2 domains, the first binding the coenzyme, often NAD, and the second binding the substrate. This latter domain determines the substrate specificity and contains amino acids involved in catalysis. Little sequence similarity has been found in the coenzyme binding domain although there is a large degree of structural similarity, and it has therefore been suggested that the structure of dehydrogenases has arisen through gene fusion of a common ancestral coenzyme nucleotide sequence with various substrate specific domains.

The NOV1 nucleic acid of the invention encoding an Androgen-Regulated Short-Chain Dehydrogenase/Reductase-like protein includes the nucleic acid whose sequence is provided in Table 1A, or a fragment thereof. The invention also includes a mutant or variant nucleic acid any of whose bases may be changed from the corresponding base shown in Table 1A while still encoding a protein that maintains its Androgen-Regulated Short-Chain Dehydrogenase/Reductase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 27% of the residues may be so changed.

The NOV1 protein of the invention includes the Androgen-Regulated Short-Chain Dehydrogenase/Reductase-like protein whose sequence is provided in Table 1B. The invention also includes a mutant or variant protein any of whose residues may be changed from the corresponding residue shown in Table 1B while still encoding a protein that maintains its Androgen-Regulated Short-Chain Dehydrogenase/Reductase-like activities and physiological functions, or a functional fragment thereof. In the mutant or variant protein, up to about 25% of the bases may be so changed.

The NOV1 nucleic acids and proteins of the invention are useful in potential diagnostic and therapeutic applications implicated in various diseases and disorders described below and/or other pathologies. For example, the compositions of the present invention will have efficacy for treatment of patients suffering from: diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, Von Hippel-Lindau (VHL) syndrome, pancreatitis, obesity, tuberous sclerosis, fertility and other diseases, disorders and conditions of the like.

NOV1 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV1 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV2

NOV2 includes two novel Insulysin-like proteins disclosed below. The disclosed proteins have been named NOV2a and NOV2b.

NOV2a

A disclosed NOV2a nucleic acid of 4321 nucleotides (also referred to as CG57433-01) encoding a novel Insulysin-like protein is shown in Table 2A. An open reading frame was identified beginning with an ATG initiation codon at nucleotides 58-60 and ending with a TGA codon at nucleotides 3115-3117. Putative untranslated regions, if any, upstream from the initiation codon and downstream from the termination codon are underlined in Table 2A, and the start and stop codons are in bold letters.

TABLE 2A
NOV2a nucleotide sequence (SEQ ID NO:3).

CCGGCTCGAAGCGCAACGAGGAAGCGTTTGCGGTGATCCCGGCGACTGCG
CTGGCTAATGCGGTACCGGCTAGCGTGGCTTCTGCACCCCGCACTGCCCA
GCACCTTCCGCTCAGTCCTCGGCGCCCGCCTGCCGCCTCCGGAGCGCCTG
TGTGGTTTCCAAAAAAAGACTTACAGCAAAATGAATAATCCAGCCATCAA
GAGAATAGGAAATCACATTACCAAGTCTCCTGAAGACAAGCGAGAATATC
GAGGGCTAGAGCTGGCCAATGGTATCAAAGTACTTCTTATCAGTGATCCC
ACCACGGATAAGTCATCAGCAGCACTTGATGTGCACATAGGTTCATTGTC
GGATCCTCCAAATATTGCTGGCTTAAGTCATTTTTGTGAACATATGCTTT
TTTTGGGAACAAAGAAATACCCTAAAGAAAATGAATACAGCCAGTTTCTC
AGTGAGCATGCAGGAAGTTCAAATGCCTTTACTAGTGGAGAGCATACCAA
TTACTATTTTGATGTTTCTCATGAACACCTAGAAGGTGCCCTAGACAGGT
TTGCACAGTTTTTTCTGTGCCCCTTGTTCGATGAAAGTTGCAAAGACAGA
GAGGTGAATGCAGTTGATTCAGAACATGAGAAGAATGTGATGAATGATGC
CTGGAGACTCTTTCAATTGGAAAAAGCTACAGGGAATCCTAAACACCCCT
TCAGTAAATTTGGGACAGGTAACAAATATACTCTGGAGACTAGACCAAAC
CAAGAAGGCATTGATGTAAGACAAGAGCTACTGAAATTCCATTCTGCTTA
CTATTCATCCAACTTAATGGCTGTTTGTGTTTTAGGTCGAGAATCTTTAG
ATGACTTGACTAATCTGGTGGTAAAGTTATTTTCTGAAGTAGAGAACAAA
AATGTTCCATTGCCAGAATTTCCTGAACACCCTTTCCAAGAAGAACATCT
TAAACAACTTTACAAAATAGTACCCATTAAAGATATTAGGAATCTCTATG
TGACATTTCCCATACCTGACCTTCAGAAATACTACAAATCAAATCCTGGT
CATTATCTTGGTCATCTCATTGGGCATGAAGGTCCTGGAAGTCTGTTATC
AGAACTTAAGTCAAAGGGCTGGGTTAATACTCTTGTTGGTGGGCAGAAGG
AAGGAGCCCGAGGTTTTATGTTTTTTATCATTAATGTGGACTTGACCGAG
GAAGGATTATTACATGTTGAAGATATAATTTTGCACATGTTTCAATACAT
TCAGAAGTTACGTGCAGAAGGACCTCAAGAATGGGTTTTCCAAGAGTGCA
AGGACTTGAATGCTGTTGCTTTTAGGTTTAAAGACAAAGAGAGGCCACGG
GGCTATACATCTAAGATTGCAGGAATATTGCATTATTATCCCCTAGAAGA
GGTGCTCACAGCGGAATATTTACTGGAAGAATTTAGACCTGACTTAATAG
AGATGGTTCTCGATAAACTCAGACCACAAAATGTCCGGGTTGCCATAGTT
TCTAAATCTTTTGAAGGAAAAACTGATCGCACAGAAGAGTGGTATGGAAC
CCAGTACAAACAAGAAGCTATACCGGATGAAGTCATCAAGAAATGGCAAA
ATGCTGACCTGAATGGGAAATTTAAACTTCCTACAAAGAATGAATTTATT
CCTACGAATTTTGAGATTTTACCGTTAGAAAAAGAGGCGACACCATACCC
TGCTCTTATTAAGGATACAGCTATGAGCAAACTTTGGTTCAAACAAGATG
ATAAGTTTTTTTTGCCGAAGGCTTGTCTCAACTTTGAATTTTTCAGTCGC
TACATTTATGCTGATCCTCTCCATTGCAACATGACATACCTGTTTATCAG
GTTATTGAAGGATGATTTAAAAGAGTATACATATGCAGCACGCCTCTCAG
GTTTGAGCTATGGCATTGCATCAGGAATGAATGCAATACTTCTTTCAGTG
AAAGGTTACAATGACAAGCAGCCAATTTTACTAAAGAAGATTATTGAGAA
AATGGCTACCTTTGAGATTGATGAAAAAAGATTTGAAATTATCAAAGAAG
CATATATGCGATCTCTTAACAATTTCCGGGCTGAACAGCCTCACCAGCAT
GCCATGTACTACCTCCGCTTGCTGATGACTGAAGTGGCCTGGACTAAAGA
TGAGTTAAAAGAAGCTCTGGATGATGTAACCCTTCCTCGCCTTAAGGCCT
TCATACCTCAGCTCCTGTCACGGCTGCACATTGAAGCCCTTCTCCATGGA
AACATAACAAAGCAGGCTGCATTAGGAATTATGCAGATGGTTGAAGACAC
CCTCATTGAACATGCTCATACCAAACCTCTCCTTCCAAGTCAGCTGGTTC
GGTATAGAGAAGTTCAGCTCCCTGACAGAGGATGGTTTGTTTATCAGCAG
AGAGATGAAGTTCACAATAACTGTGGCATCGAGATATACTACCAAACAGA
CATGCAAAGCACCTCAGAGAATATGTTTCTGGAGCTCTTCTGTCAGATTA
TCTCGGAACCTTGCTTCAACACCCTGCGCACCAAGGAGCAGTTGGGCTAT
ATCGTCTTCAGCGGGCCACGTCGAGCTAATGGCATACAGGGCTTGAGATT
CATCATCCAGTCAGAAAAGCCACCTCACTACCTAGAAAGCAGAGTGGAAG
CTTTCTTAATTACCATGGAAAAGTCCATAGAGGACATGACAGAAGAGGCC
TTCCAAAAACACATTCAGGCATTAGCAATTCGTCGACTAGACAAACCAAA
GAAGCTATCTGCTGAGTGTGCTAAATACTGGGGAGAAATCATCTCCCAGC
AATATAATTTTGACAGAGATAACACTGAGGTTGCATATTTAAAGACACTT
ACCAAGGAAGATATCATCAAATTCTACAAGCAAATGTTGGCAGTAGATGC
TCCAAGGAGACATAAGGTATCCGTCCATGTTCTTGCCAGGGAAATGGATT
CTTGTCCTGTTGTTGGAGAGTTCCCATGTCAAAATGACATAAATTTGTCA
CAAGCACCAGCCTTGCCACAACCTGAAGTGATTCAGAACATGACCGAATT
CAAGCGTGGTCTGCCACTGTTTCCCCTTGTGAAACCACATATTAACTTCA
TGGCTGCAAAACTCTGAAGATTCCCCATGCATGGGAAACTGCAAGTGGAT
GCATTCCTGAGTCTTCCAGAGCCTAAGAAAATCATCTTGGCCACTTTAAT
AGTTTCTGATTCACTATTAGAGAAACAAACAAAAAATTGTCAAATGTCAT
TATGTAGAAATATTATAAATCCAAAGTAAATTACAAAATCTTATAGATGT
AGAATATTTTTTAAATACATGCCTCTTAAATATTTTAAAATTTTTCTTTT
GATTACTGAGAGAAATTTCCCCAATATAACAATGCTTAAAATGAATGATA
TTCCTATAGAATCTTCCTTCCCTATTCTGTAAAATAGTCACTTGTCCGAA
GAAAGTTAAAAGTTAGCTCTTTTCTAAAGCCTCCTAGCTTGACATAGAAG
GCTTCACAACATTTAGAAAGGTAATAAACTTTTTAAAAAATTGATCCTCA
ATTTGCTTTCTACTTGATGGTTTCATGTAAATCAGTGGAAAACATTACAT
TTGGCAGATGATAAAGCAATGTCATCTTTTATTAGTGAAATGCTGGTTAT
ATAAGGCATGGTTTTAATCTTTTTATAAAATTTGAACATGTTTTTTATGC
CAACTCGTAAAATGCTAGAAAACCCTACTTATTTACAATGCTAGAAATAC
AGACTTACCTTACATCAATTTTGTCCTAAACCGAATTTCTCAGGATTACT
GTGGTTTCTTTCATTCTGATTGAATTATATTGACCTACTTCTTCATAGTT
GGTTTGCAGTGTTCCATGAGTTTTACTTTTCCTCATCAACATATTGCTTT
AACACAACATATTTATTTAACACGTACAAATAGGGTCAACTTCAGATCCT
ACTGAGTGTGTGACATGCTTTTCCAACATCAGCTTTTTGTAACCACCTGT
ATAACTTTTTATTACAGTGAAATTGCAGTCAGTATGTGAACCAAAATATC
TTGCCCCTTTATGAATTTAAAGGCAGCCAATACAAAGCCACCTTTTTGGA
AAATATAAAAAGTAAAGCCTTGCATTCTTATATAGCAGGTCTTCATAAAA
CTCTAAAATCCCTTGTTGCTACCAGTCTAATCTTGCCTTAAATGTTAAGT
TATTTTTTGAATATATAAATATAAACATATAAACACAGATGATGACTGGA
GTAGACTTTTAAAAAAATATTTTTTTCATGAGATACTATTTTAGGTGAAA
TTGTACTGTAGATTTACAGCT

The disclosed NOV2a nucleic acid sequence, localized to chromsome 10, has 3265 of 3336 bases (97%) identical to a gb:GENBANK-ID:HUMIDE|acc:M21188.1 mRNA from Homo sapiens (Human insulin-degrading enzyme (IDE) mRNA, complete cds) (E=0.0).

A NOV2a polypeptide (SEQ ID NO:4) encoded by SEQ ID NO:3 has 1019 amino acid residues and is presented using the one-letter-code in Table 2B. Signal P, Psort and/or Hydropathy results predict that NOV2a contains a signal peptide and is likely to be localized to the peroxisome (microbody) with a certainty of 0.8347 or the mitochondrial matrix space with a certainty of 0.6517. The most likely cleavage site for a NOV2a peptide is between amino acids 22 and 23: VLG-AR.

TABLE 2B
Encoded NOV2a protein sequence (SEQ ID NO:4).

MRYRLAWLLHPALPSTFRSVLGARLPPPERLCGFQKKTYSKMNNPAIKRI
GNHITKSPEDKREYRGLELANGIKVLLISDPTTDKSSAALDVHIGSLSDP
PNIAGLSHFCEHMLFLGTKKYPKENEYSQFLSEHACSSNAFTSGEHTNYY
FDVSHEHLEGALDRFAQFFLCPLFDESCKDREVNAVDSEHEKNVMNDAWR
LFQLEKATGNPKHPFSKFGTGNKYTLETRPNQEGIDVRQELLKFHSAYYS
SNLMAVCVLGRESLDDLTNLVVKLFSEVENKNVPLPEFPEHPFQEEHLKQ
LYKTVPIKDIRNLYVTFPIPDLQKYYKSNPGHYLGHLIGHEGPGSLLSEL
KSKGWVNTLVGGQKEGARCFMFFIINVDLTEECLLHVEDIILHMFQYIQK
LRAEGPQEWVFQECKDLNAVAFRFKDKERPRGYTSKIAGILHYYPLEEVL
TAEYLLEEFRPDLIEMVLDKLRPENVRVAIVSKSFEGKTDRTEEWYGTQY
KQEAIPDEVIKKWQNADLNGKFKLPTKNEFIPTNFEILPLEKEATPYPAL
IKDTAMSKLWFKQDDKFFLPKACLNFEFFSRYTYADPLHCNMTYLFIEUL
KDDLKEYTYAARLSGLSYGIASGMNAILLSVKGYNDKQPILLKKIIEKMA
TFEIDEKRFEIIKEAYMRSLNNFRAEQPHQHAMYYLRLLMTEVAWTKDEL
KEALDDVTLPRUKAFTPQLLSRLHIEALLHGNITKQAALGIMQMVEDTLI
EHAHTKPLLPSQLVRYREVQLPDRGWFVYQQRDEVHNNCGIEIYYQTDMQ
STSENMFLELFCQIISEFCFNTLRTKEQLGYIVFSGPRRANGIQCLRFII
QSEKPPHYLESRVEAFLITMEKSIEDMTEEAFQKHIQALAIRRLDKPKKL
SAECAKYWGETISQQYNFDRDNTEVAYLKTLTKEDIIKFYKEMUAVDAPR
RHKVSVHVLAREMDSCPVVGEFPCQNDINLSQAPALPQPEVIQNMTEFKR
GLPLFPLVKPHINFMAAKL

The NOV2a amino acid sequence has 989 of 1019 amino acid residues (97%) identical to, and 999 of 1019 amino acid residues (98%) similar to, the 1019 amino acid residue ptnr:pir-id:SNHUIN protein from human (insulysin (EC 3.4.24.56)) (E=0.0).

The disclosed NOV2a is expressed in at least the following tissues: Cervix, Liver, Spleen, Testis, Melanocyte, Heart and Uterus. This information was derived by determining the tissue sources of the sequences that were included in the invention including but not limited to SeqCalling sources, Public EST sources, Literature sources, and/or RACE sources.

NOV2b

A disclosed NOV2b nucleic acid of 3153 nucleotides (also referred to as CG57433-02) encoding a novel Insulysin-like protein is shown in Table 2C. An open reading frame was identified beginning with an ATG initiation codon at nucleotides 37-39 and ending with a TGA codon at nucleotides 3094-3096. Putative untranslated regions, if any, upstream from the initiation codon and downstream from the termination codon are underlined in Table 2C, and the start and stop codons are in bold letters.

TABLE 2C
NOV2b nucleotide sequence (SEQ ID NO:5).

AAGCGTTTGCGGTGATCCCGGCGACTGCCCTGGCTAATGCGGTACCGGCT
AGCGTGGCTTCTGCACCCCGCACTGCCCAGCACCTTCCGCTCAGTCCTCG
GCGCCCGCCTGCCGCCTCCGGAGCGCCTCTGTGGTTTCCAAAAAAAGACT
TACAGCAAAATGAATAATCCAGCCATCAAGAGAATAGCAAATCACATTAC
CAAGCCTCCTGAAGACAAGCGAGAATATCGAGGGCTAGAGCTGGCCAATG
GTATCAAACTACTTCTTATCAGTGATCCCACCACGGATAAGTCATCAGCA
GCACTTGATGTGCACATAGGTTCATTCTCCGATCCTCCAAATATTGCTGG
CTTAAGTCATTTTTGTGAACATATCCTTTTTTTGGGAACAAAGAAATACC
CTAAAGAAAATGAATACAGCCAGTTTCTCAGTGAGCATGCAGGAAGTTCA
AATGCCTTTACTAOTGGAGAGCATACCAATTACTATTTTGATGTTTCTCA
TGAACACCTAGAAGGTGCCCTAGACAGCTTTGCACAGTTTTTTCTGTGCC
CCTTGTTCGATGAAAGTTGCAAAGACAGACAGGTGAATGCAGTTGATTCA
GAACATGAGAAGAATCTCATCAATCATGCCTGGAGACTCCTTCAATTGCA
AAAAGCTACAGCGAATCCTAAACACCCCTTCAGTAAATTTGGCACAGGTA
ACAAATATACTCTGGAGACTAGACCAAACCAAGAAGGCATTGATGTAAGA
CAAGACCTACTGAAATTCCATTCTGCTTACTATTCATCCAACTTAATCGC
TCTTTGTGTTTTAGGTCGACAATCTTTAGATGACTTGACTAATCTGGTGG
TAAAGTTATTTTCTGAAGTAGAGAACAAAAATGTTCCATTGCCAGAATTT
CCTGAACACCCTTTCCAAGAAGAACATCTTAAACAACTTTACAAAATAGT
ACCCATTAAAGATATTAGGAATCTCTATGTGACATTTCCCATACCTGACC
TTCAGAAATACTACAAATCAAATCCTGGTCATTATCTTGCTCATCTCATT
GGGCATGAAGGTCCTGGAAGTCTGTTATCAGAACTTAAGTCAAAGGGCTG
GGTTAATACTCTTGTTGGTGGGCAGAAGGAAGGAGCCCGAGGTTTTATGT
TTTTTATCATTAATGTGGACTTGACCGAGGAAGGATTATTACATGTTGAA
GATATAATTTTGCACATGTTTCAATACATTCAGAGGTTACGTGCAGAAGG
ACCTCAAGAATGGGTTTTCCAAGAGTGCAAGGACTTGAATGCTGTTACTT
TTAGGTTTAAAGACAAAGAGAGGCCACGGGGCTATACATCTAAGATTGCA
GGAATATTGCATTATTATCCCCTAGAAGAGGTGCTCACAGCGGAATATTT
ACTGGAAGAATTTAGACCTGACTTAATAGAGATGGTTCTCGATAAACTCA
GACCAGAAAATGTCCGGGTTGCCATAGTTTCTAAATCTTTTGAAGGAAAA
ACTGATCGCACAGAAGAGTGGTATGGAACCCAGTACAAACAAGAAGCTAT
ACCGGATGAAGTCATCAAGAAATGGCAAAATGCTGACCTGAATGGGAAAT
TTAAACTTCCTACAAAGAATGAATTTATTCCTACGAATTTTGAGATTTTA
CCGTTAGAAAAAGAGGCGACACCATACCCTGCTCTTATTAAGGATACAGC
TATGAGCAAACTTTGGTTCAAACAAGATGATAAGTTTTTTTTGCCGAAGG
CTTGTCTCAACTTTGAATTTTTCAGTCGCTACATTTATGCTGATCCTCTC
CATTGCAACATGACATACCTGTTTATCAGGTTATTGAAGGATGATTTAAA
AGAGTATACATATGCAGCACGCCTCTCAGGTTTGAGCTATGGCATTGCAT
CAGGAATGAATGCAATACTTCTTTCAGTGAAAGGTTACAATGACAAGCAG
CCAATTTTACTAAAGAAGATTATTGAGAAAATGGCTACCTTTGACATTGA
TGAAAAAAGATTTGAAATTATCAAGAAGCATATATGCGATCTCTTAACAA
TTTCCGGGCTGAACAGCCTCACCAAGCATGCCATGTACTACCTCCGCTTG
CTGATGACTGAAGTGGCCTGGACTAAAOATGAGTTAAAAGAGGCTCTGGA
TGATGTAACCCTTCCTCGCCTTAAGGCCTTCATACCTCAGCTCCTGTCAC
GGCTGCACATTGAAGCCCTTCTCCATGGAAACATAACAAAGCAGGCTGCA
TTAGGAATTATGCAGATGGTTGAAGACACCCTCATTGAACATGCTCATAC
CAAACCTCTCCTTCCAAGTCAGCTGGTTCGGTATAGAGAAGTTCAGCTCC
CTGACAGAGGATGGTTTGTTTATCAGCAGAGAAATGAAGTTCACAATAAC
TGTGGCATCGAGATATACTACCAAACAGACATGCAAAGCACCTCAGAGAA
TATGTTTCTGGAGCTCTTCTGTCAGATTATCTCGGAACCTTGCTTCAACA
CCCTGCGCACCAAGGAGCAGTTGGGCTATATCGTCTTCAGCGGGCCACGT
CGAGCTAATGGCATACAGGGCTTGAGATTCATCATCCAGTCAGAAAAGCC
ACCTCACTACCTAGAAAGCAGAGTGGAAGCTTTCTTAATTACCATGGAAA
AGTCCATAGAGGACATGACAGAAGAGGCCTTCCAAAAACACATTCAGGCA
TTAGCAATTCGTCGACTACACAAACCAAAGAAGCTATCTGCTGAGTGTGC
TAATACTGGGGAGAATCATCTCCCAGCAATATAATTTTTTGACAGAGATA
ACACTGAGGTAGCATATTTAAAGACACTTACCAAGGAAGATATCATCAAA
TTCTACAAGGAAATGTTGGCAGTAGATGCTCCAAGCAGACATAAGGTATC
CGTCCATGTTCTTGCCAGGGAAATGGATTCTTGTCCTGTTGTTGGAGAGT
TCCCATGTCAAATGACATAAATTTGTCACAAGCACCAGCCTTGCCACAAC
CTGAAAGTGATTCAGAACATGACCGAATTCAAGCGTGGTCTGCCACTGTT
TCCCCTTGTGAAACCACATATTAACTTCATGGCTGCAAAACTCTGAAGAT
TCCCCATGCATGGGAAAGTGCAAGTGGATGCATTCCTGAGTCTTCCAGAG
CCT

The disclosed NOV2b nucleic acid sequence, localized to chromsome 10q23-q25, 3078 of 3153 bases (97%) identical to a gb:GENBANK-ID:HUMIDE|acc:M21188.1 mRNA from Homo sapiens (Human insulin-degrading enzyme (IDE) mRNA, complete cds) (E=0.0).

A NOV2b polypeptide (SEQ ID NO:6) encoded by SEQ ID NO:5 has 1019 amino acid residues and is presented using the one-letter code in Table 2D. Signal P, Psort and/or Hydropathy results predict that NOV2b contains a signal peptide and is likely to be localized to the peroxisome (microbody) with a certainty of 0.8347 or the mitochondrial matrix space with a certainty of 0.6517. The most likely cleavage site for a NOV2b peptide is between amino acids 22 and 23: VLG-AR.

TABLE 2D
Encoded NOV2b protein sequence (SEQ ID NO:6).

MRYRLAWLLHPALPSTFRSVLGARLPPPERLCGFQKKTYSKThNPAIKRI
GNHITKPPEDKREYRGLELANGIKVLLISDPTTDKSSAALDVHIGSLSDP
PNIAGLSHFCEHMLFLGTKKYPKENEYSQFLSEHAGSSNAFTSGEHTNYY
FDVSHEHLECALDRFAQFFLCPLFDESCKDREVNAVDSEHEKNVMNDAWR
LLQLEKATGNPKHPFSKFGTGNKYTLETRPNQEGIDVRQELLKFHSAYYS
SNLMAVCVLGRESLDDLTNLVVKLFSEVENKNVPLPEFPEHPFQEEHLKQ
LYKIVPIKDIRNLYVTFPIPDLQKYYKSNPGHYLGHLIGHEGPGSLLSEL
KSKGWVNTLVGGQKEGARGEMFFIINVDLTEEGLLHVEDIILHMFQYIQR
LRAEGPQEWVFQECKDLNAVTFRFKDKERPRGYTSKIAGILHYYPLEEVL
TAEYLLEETRPDLIEMVLDKLRPENVRVAIVSKSFEGKTDRTEEWYGTQY
KQEAIPDEVIKKWQNADLNGKFKLPTKNEFIPTNFEILPLEKEATPYPAL
IKDTAMSKLWFKQDDKFFLPKACLNFEFFSRYIYADPLHCNMTYLFIRLL
KDDLKEYTYAARLSGLSYGIASGMNAILLSVKGYNDKQPILLKKITEKMA
TFEIDEERFEIIKEAYMRSLNNFRAEQPHQHAMYYLRLLMTEVAWTKDEL
KEALDDVTLPRLKAFIPQLLSRLHIEALLHGNITKQALGIMQMVEDTLIE
HAHTKPLALPSQLVRYREVQLPDRGWFVYQQRNEVHNNCGIEIYYQTDMQ
STSENMFLELFCQIISEPCFNTLRTKEQLGYIVFSGPRRANGIQGLRFII
QSEKPPHYLESRVEAFLITMEKSIEDMTEEAFQKHIQALAIRRLDKPKKL
SAECAKYWGEIISQQYNFDRDNTEVAYLKTLTKEDIIKFYKEMLAVDAPR
RHKVSVHVLAREMDSCPVVGEFPCQNDINLSQAPALPQPEVIQNMTEFKR
GLPLFPLVKPHINFMAAKL

The NOV2b amino acid sequence has 986 of 1019 amino acid residues (96%) identical to, and 996 of 1019 amino acid residues (97%) similar to, the 1019 amino acid residue ptnr:pir-id:SNHUIN protein from human (insulysin (EC 3.4.24.56)) (E=0.0).

The disclosed NOV2b is expressed in at least the following tissues: Lung, skin, uterus, colon, breast, liver, spleen, testis, and pediatric pre-B cell acute lymphoblastic leukemia. This information was derived by determining the tissue sources of the sequences that were included in the invention including but not limited to SeqCalling sources, Public EST sources, Literature sources, and/or RACE sources.

Possible SNPs found for NOV2a are listed in Table 2E and possible SNPs found for NOV2b are limited in Table 2F.

TABLE 2E
SNPs

| | | | Amino | |
|---|---|---|---|---|
| | Nucleotide | Base | Acid | Base |
| Variant | Position | Change | Position | Change |
| 13375689 | 177 | C > T | Silent | N/A |
| 13375688 | 184 | A > G | 43 | Asn > Asp |
| 13375672 | 223 | A > T | 56 | Lys > End |
| 13375687 | 226 | T > C | 57 | Ser > Pro |
| 13375686 | 270 | T > C | Silent | N/A |
| 13375685 | 322 | G > T | 89 | Ala > Ser |
| 13375684 | 330 | T > C | Silent | N/A |
| 13375683 | 355 | C > T | 100 | Pro > Ser |
| 13375682 | 435 | A > G | Silent | N/A |
| 13375681 | 450 | C > T | Silent | N/A |
| 13375326 | 661 | T > C | 202 | Phe > Leu |
| 13375680 | 685 | A > G | 210 | Asn > Asp |
| 13375679 | 905 | T > C | 283 | Val > Ala |
| 13375327 | 1256 | A > G | 400 | Lys > Arg |
| 13375328 | 1318 | G > A | 421 | Ala > Thr |
| 13375329 | 1412 | C > T | 452 | Ala > Val |
| 13375678 | 2686 | A > G | 877 | Met > Val |
| 13375677 | 2751 | G > A | Silent | N/A |
| 13375676 | 2815 | A > G | 920 | Arg > Gly |
| 13375675 | 2895 | A > G | Silent | N/A |
| 13375674 | 2910 | A > G | Silent | N/A |
| 13375673 | 2984 | A > G | 976 | Asn > Ser |
| 13377049 | 3077 | T > A | 1007 | Leu > His |

TABLE 2F
SNPs

| Consensus | | Base | |
|---|---|---|---|
| Position | Depth | Change | PAF |
| 656 | 7 | C > T | 0.429 |

NOV2a and NOV2b share high degree of homology. Homologies to any of the above NOV2 proteins will be shared by the other NOV2 proteins insofar as they are homologous to each other as shown above. Any reference to NOV2 is assumed to refer to both of the NOV2 proteins in general, unless otherwise noted.

NOV2a has homology to the amino acid sequences shown in the BLASTP data listed in Table 2H.

TABLE 2H
BLAST results for NOV2a

| Gene Index/ | Protein/ | Length | Identity | Positives | |
|---|---|---|---|---|---|
| Identifier | Organism | (aa) | (%) | (%) | Expect |
| gi\|4826770\|ref\|NP_004960.1\| | insulysin; | 1019 | 974/1019 | 984/1019 | 0.0 |
| (NM_004969) | insulinase | | (95%) | (95%) | |
| | [Homo | | | | |
| | sapiens] | | | | |
| gi\|6981076\|ref\|NP_037291.1\| | insulin | 1019 | 934/1019 | 965/1019 | 0.0 |
| (NM_013159) | degrading | | (91%) | (94%) | |
| | enzyme | | | | |
| | [Rattus | | | | |
| | norvegicus] | | | | |
| gi\|13621162\|ref\|NP_112419.1\| | insulin | 1019 | 929/1019 | 963/1019 | 0.0 |
| (NM_031156) | degrading | | (91%) | (94%) | |
| | enzyme [Mus | | | | |
| | musculus] | | | | |
| gi\|18576366\|ref\|XP_051153.2\| | insulysin | 554 | 530/554 | 539/554 | 0.0 |
| (XM_051153) | [Homo | | (95%) | (96%) | |
| | sapiens] | | | | |
| gi\|7296294\|gb\|AAF51584.1\| | Ide gene | 990 | 439/966 | 634/966 | 0.0 |
| (AE003591) | product | | (45%) | (65%) | |
| | [Drosophila | | | | |
| | melanogaster] | | | | |

Table 2J lists the domain description from DOMAIN analysis results against NOV2a. This indicates that the NOV2a sequence has properties similar to those of other proteins known to contain these domains.

TABLE 2J

Domain Analysis of NOV2a
gnl|Pfam|pfam00675, Peptidase_M16, Insulinase (Peptidase family M16).
(SEQ ID NO:67)
CD-Length = 149 residues, 99.3% aligned
Score = 157 bits (396), Expect = 4e−39

| | |
|---|---|
| Query: 74 | KVLLISDPTTDKSSAALDVHIGSLSDPPNIAGLSHFCEHMLFLGTKKYPKENEYSQFLSE 133 |
| | +\| \| \|\| \| \|+ \| \| \|\| +\| + \|\|+\|\| \|\|\| \| \|\|\|\|\|\| \|\| + \| + |
| Sbjct: 1 | RVALEHDPPADTSAVGLWVDAGSRYEPDDNNGLAHFLEHMAFKGTKKYP-SNELEEELEK 59 |
| Query: 134 | HACSSNAFTSGEHTNYYFDVSHEHLEGALDRFAQFFLCPLFDESCKDR-------EVNAV 186 |
| | \|\| \|\|+\|\| \|\|\| \|\| +\| ++ \| \|+\|\| \| \|\|\| \|\|\| \| +\| \|\| \|\| |
| Sbjct: 60 | LGGSLNAYTSREHTAYYVEVLNDDLPKAVDRLADFFLNPLFSPSEVERERLVVLYEVEAV 119 |
| Query: 187 | DSEHEKNVMN----DAWRLPQLEKATGNP 211 |
| | \|+\| + +++ \|+\| \| ++ \| |
| Sbjct: 120 | DAEPQAVLLDNLHAAAYRGTPLGRSLLGP 148 |

After binding to its receptor on the cell surface, insulin is internalized by receptor-mediated endocytosis and degraded within components of the endosomal apparatus. Degradation of insulin is important in the termination of signaling and clearance of the circulating hormone. It has been proposed that insulin-degrading enzyme (IDE), an evolutionarily conserved, neutral thiol-metalloendopeptidase, plays a crucial role in the degradation of internalized insulin in many types of cells. Despite the substantial evidence supporting the importance of IDE in cellular insulin degradation, there is controversy over its mode and site of action, mainly because of its cytosolic location. Its physiological location in cells has recently been elucidated through subcellular fractionation of liver parenchyma and through immunofluorescence microscopy of stably transfected Chinese hamster ovary cells that overexpress IDE. These experiments have excluded the presence of the enzyme in endosomes and have defined a peroxisomal location, consistent with the presence of a peroxisomal targeting sequence at the carboxyl terminus of the protein. Recently, researchers have demonstrated the functional significance of peroxisome-associated IDE (type I peroxisomal enzyme) in degrading cleaved leader peptides of peroxisomal proteins targeted by the type II motif. IDE is the first cloned and characterized proteinase to be localized to peroxisomes. Moreover, IDE appears to be a member of a newly identified superfamily of metalloendopeptidases that has an HXXEH active-site motif. Although fundamental questions concerning the biological role of IDE remain, its high degree of evolutionary conservation suggests that it must have important functions and multifaceted biological significance (Authier et al., Clin Invest Med 19 (3):149-60, 1996)

Insulin degradation is a regulated process that plays a role in controlling insulin action by removing and inactivating the hormone. Abnormalities in insulin clearance and degradation are present in various pathological conditions including type 2 diabetes and obesity and may be important in producing clinical problems. The uptake, processing, and degradation of insulin by cells is a complex process with multiple intracellular pathways. Most evidence supports IDE as the primary degradative mechanism, but other systems (PDI, lysosomes, and other enzymes) undoubtedly contribute to insulin metabolism. Recent studies support a multifunctional role for IDE, as an intracellular binding, regulatory, and degradative protein. IDE increases proteasome and steroid hormone receptor activity, and this activation is reversed by insulin. This raises the possibility of a direct intracellular interaction of insulin with IDE that could modulate protein and fat metabolism. The recent findings would place intracellular insulin-IDE interaction into the insulin signal transduction pathway for mediating the intermediate effects of insulin on fat and protein turnover (Duckworth et al., Endocr Rev 19 (5):608-24, 1998).

A number of proteases dependent on divalent cations for their activity have been shown to belong to one family, on the basis of sequence similarity. These enzymes include those listed below.

Insulinase (EC 3.4.24.56) (also known as insulysin or insulin-degrading enzyme or IDE), a cytoplasmic enzyme which seems to be involved in the cellular processing of insulin, glucagon and other small polypeptides.

Escherichia coli protease III (EC 3.4.24.55) (pitrilysin) (gene ptr), a periplasmic enzyme that degrades small peptides.

Mitochondrial processing peptidase (EC 3.4.24.64) (MPP). This enzyme removes the transit peptide from the precursor form of proteins imported from the cytoplasm across the mitochondrial inner membrane. It is composed of two nonidentical homologous subunits termed alpha and beta. The beta subunit seems to be catalytically active while the alpha subunit has probably lost its activity.

Nardilysin (EC 3.4.24.61) (N-arginine dibasic convertase or NRD convertase) this mammalian enzyme cleaves peptide substrates on the N-terminus of Arg residues in dibasic stretches.

Klebsiella pneumoniae protein pqqF. This protein is required for the biosynthesis of the coenzyme pyrrolo-quinoline-quinone (PQQ). It is thought to be protease that cleaves peptide bonds in a small peptide (gene pqqA) thus providing the glutamate and tyrosine residues necessary for the synthesis of PQQ.

Yeast protein AXL1, which is involved in axial budding.

Eimeria bovis sporozoite developmental protein.

Escherichia coli hypothetical protein yddC and HI1368, the corresponding Haemophilus influenzae protein.

Bacillus subtilis hypothetical protein ymxG.—Caenorhabditis elegans hypothetical proteins C28F5.4 and F56D2.1.

It should be noted that in addition to the above enzymes, this family also includes the core proteins I and II of the mitochondrial bc1 complex (also called cytochrome c reductase or complex III), but the situation as to the activity or lack of activity of these subunits is quite complex. In mammals and yeast, core proteins I and II lack enzymatic activity. In Neurospora crassa and in potato core protein I is equivalent to the beta subunit of MPP. In Euglena gracilis, core protein I seems to be active, while subunit II is inactive.

These proteins do not share many regions of sequence similarity; the most noticeable is in the N-terminal section. This region includes a conserved histidine followed, two residues later by a glutamate and another histidine. In pitrilysin, it has been shown that this H-x-x-E-H motif is involved in enzyme activity; the two histidines bind zinc and the glutamate is necessary for catalytic activity. Non active members of this family have lost from one to three of these active site residues. A signature pattern has been developed that detect active members of this family as well as some inactive members.

The NOV2 nucleic acid of the invention encoding an Insulysin-like protein includes the nucleic acid whose sequence is provided in Tables 2A and 2C, or a fragment thereof. The invention also includes a mutant or variant nucleic acid any of whose bases may be changed from the corresponding base shown in Tables 2A and 2C while still encoding a protein that maintains its Insulysin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3% of the residues may be so changed.

The NOV2 protein of the invention includes the Insulysin-like protein whose sequence is provided in Tables 2B and 2D. The invention also includes a mutant or variant protein any of whose residues may be changed from the corresponding residue shown in Tables 2B and 2D while still encoding a protein that maintains its Insulysin-like activities and physiological functions, or a functional fragment thereof. In the mutant or variant protein, up to about 4% of the bases may be so changed.

The NOV2 nucleic acids and proteins of the invention are useful in potential diagnostic and therapeutic applications implicated in various diseases and disorders described below and/or other pathologies. For example, the compositions of the present invention will have efficacy for treatment of patients suffering from: Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Endometriosis, Fertility, Von Hippel-Lindau (VHL) syndrome, Cirrhosis, Transplantation, Hemophilia, Hypercoagulation, Idiopathic thrombocytopenic purpura, Immunodeficiencies, Retinitis pigmentosa, autosomal dominant; Retinitis pigmentosa, autosomal recessive; SEMD, Pakistani type; Urofacial syndrome; Alzheimer disease 6; Cholesteryl ester storage disease; Corneal dystrophy, Thiel-Behnke type; Dubin-Johnson syndrome; Leukemia, T-cell acute lymphocytic; Leukemia, T-cell acute lymphocytic; Spinocerebellar ataxia, infantile-onset, with sensory neuropathy; Split hand/foot malformation, type 3; Tolbutamide poor metabolizer; Warfarin sensitivity; Wolman disease; Anterior segment mesenchymal dysgenesis and cataract; Cataract, congenital; Neurofibrosarcoma; Diabetes mellitus, insulin-dependent, 17; Diabetes mellitus, insulin-dependent, 17; obesity, insulin resistance, Graft vesus host and other diseases, disorders and conditions of the like.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV2 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV3

A disclosed NOV3 nucleic acid of 3509 nucleotides (also referred to as CG57362-01) encoding a novel BCSC-1-like protein is shown in Table 3A. An open reading frame was identified beginning with an ATG initiation codon at nucleotides 155-157 and ending with a TGA codon at nucleotides 3206-3208. Putative untranslated regions upstream from the intitation codon and downstream from the termination codon are underlined in Table 3A, and the start and stop codons are in bold letters.

TABLE 3A
NOV3 Nucleotide Sequence (SEQ ID NO:7)

GGTCCGAGCGGGGCTCTGTGACGACAGCCCCACCAGCTGCTTCGGGGTGG
GCAGCCTTCAGGAGGAAGGGCTGGCCTGGGAGGAGCTGGCTGCCCCTCGG
GACGTGTTCTCAGGCCCTGCCCGCTGCCCTGCCCCATATACCTTCTCCTT
CGAGATGCTGGTGACTGGGCCATGCCTGCTTGCAGGCCTGGAGAGCCCCT
CTCATGCTCTGCGGCCAGATGCCCCCCCTCATGCCAGCTCTGCAGCCACC
ATCTGTGTCACACTCGCAGAGGGCCACCACTGTGACCGGGCCTTGGAGAT
CCTGCTGCACCCCAGTGAGCCCCATCAGCCACACCTGATGCTGGAGGGCG
GCAGCCTGAGCTCAGCAGAATATGAGGCCCGGCTGAGGGCCCGCCGAGAT
TTTCAGAGGCTACAGCGAGGGGACAGTGATGGGGACCCGCAGGTGTGGTT
CCTGCAGCGACGCTTCCACAAGGACATCCTCCTGAACCCCGTGCTGGCGC
TGAGCTTCTGCCCAGACCTGAGCTCCAAGCCCGGACACCTGGGGACAGCT
ACTCGGGAGCTACTCTTCCTTTTGGATAGCAGCAGCGTGGCACACAAGGA
TGCCATTGTTTTGGCTGTOAAGTCCCTCCCACCCCAGACGCTTATCAACC
TGGCCGTGTTTGGGACGTTGGTGCAGCCACTCTTCCCAGAGAGCCGGCCT
TGCAGTGATGATGCTGTGCAGCTGATCTGCGAGAGCATTGAGACCCTGCA
GGTTCCGAGTGGGCCCCCAGACGTGCTGGCTGCTCTGGACTGGGCCGTGG
GGCAGCCCCAGCACAGGGCCTACCCTCGGCAGCTGTTCCTGCTCACTGCT
GCCTCACCCATGGCCGCCACTACCCACCGAACCCTGGAGCTCATGAGGTG
GCACAGGGGGACAGCCAGGTGCTTCTCCTTTGGGCTGGGGCCCACCTGCC
ACCAGCTGCTCCAGGGTTTATCTGCCCTCAGCAGCCGAGGTCGCGCCACT
GCACTCCAGCCTGGGCGACAGAGCAAGCCACAGCTGGTACAGGCTCTGCG
GAAGGCACTGGAGCCTGCTTTGAGTGACATCTCTGTGGACTGGTTTGTGC
CCGACACTGTGGAGGCACTGCTGACCCCTCGGGAGATCCCAGCACTCTAC
CCTGGGGACCAGCTGCTCGGTTACTGCTCACTCTTCAGGGTGGATGGCTT
CCGGTCCCGCCCACCAGGGGGCCAAGAGCCTGGCTGGCAGAGCTCGGGTG
GGTCCGTGTTTCCATCCCCAGAAGAGGCCCCGTCTGCTGCCAGCCCTGGC
ACTGAGCCCACTGGCACCTCAGAGCCACTGGGAACAGGCACTGTCTCAGC
AGAACTGTCCAGCCCATGGGCTGCCAGGGACTCGGAGCAGAGTGGTACTG
ATGCTCTGACAGACCCAGTCACGGATCCTGGACCCAACCCCTCTGACACA
GCCATATGGCGCCGCATCTTTCAGTCCTCGTACATTCGGGAGCAGTATGT
GCTCACCCACTGCTCTGCCAGCCCCGAGCCAGGCCCAGGCTCCACAGGCA
GCAGTGAGTCCCCAGGCTCACAGGGCCCTGGCTCCCCCGAAGGTAGTGCT
CCCTTGGAGCCCCCTTCTCAGCAGGGCTGCCGCAGTCTGGCCTGGGGAGA
ACCTGCAGGCTCCCGCTCCTGTCCCCTGCCTGCACCCACACCAGCTCCAT
TCAAGGTGGGGGCCTTGAGTACTGAGGTGCTGGGCCGTCAGCACAGAGCG
GCTCTGGCTGGCCGAAGCCTCTCATCCCCTCCAGGCCGGGCAAACCAAGT
CCCCGGCCGACCCCGGAAACCCTCTTTGGGTGCAATACTAGATGGCCCAA
GTCCTGAGCCAGGCCAACAGTTGGGACAAGGCCTGGATGACTCAGGTAAC
CTGCTCTCCCCAGCCCCTATGGACTGGGACATGCTGATGGAACCACCCTT
CTTATTCACGGCTGTCCCTCCTAGTGGGGAGTTGCCCCCTCCAGCAGTGC
CTCCCCAGGCTCCACGCTGCCATGTGGTGATCCGGGGCCTGTGTGGGGAG
CAGCCCATGTGCTGGGAGGTGGGTGTTGGGCTGGAGACACTGTGGGGACC
TGGAGATGGCTCACAGCCTCCCTCACCTCCTGTAAGAGAAGCTGCTTGGG
ACCAAGCACTCCATCGGCTGACAGCAGCCTCTGTGGTCCGGGACAATGAG
CAGCTGGCCCTCCGAGGAGGGGCAGAGACCACAGCTGACCGGGGCCATGC
CCGGAGGTGCTGGCTTCGAGCCCTTCAAACAAGTAAGGTCAGCTCTGCCC
CCTCCTGCTTCACTTGCCCTGTAGCTGTGGATGCTACTACTAGGGAGGTC
CTGCCTGGGGCCCTGCAGGTGTGCAGCTCAGAGCCCGCTGAGCCCCCAGG
AACCCCTCCTGCCTCTCACAGCCATCTAGATGCAGCTCCTCTGCCCACTG
TTGTCTACTCTAAAGCTGCCTGGGACTCGGACCAAAATGGCAACTCCAAG
CGTGCTTTGGGGGACCCTGCCACTCCCACGGAAGGTCCTCGCCGCCCACC
TCCCCGTCCTCCCTGTCGGCTCAGCATGGGCCGCCGTCACAAACTCTGTA
GCCCTGACCCGGGCCAGGCCAACAACAGTGAAGCCAGCGACCATGACTAC
CTGCCCTTGCTGCGGCTGCAGGAGGCACCAGGCTCCTTCCGCCTGGACGC
GCCCTTCTGCGCCGCTGTGCGCATCTCGCAGGAGCGCCTCTGCCGTGCCT
CGCCCTTTGCCGTGCACCGCGCCAGCCTCAGCCCCACCTCGGCCTCATTG
CCCTGGGCACTTCTGGGCCCTGGTGTTGGCCAGGGTGACAGTGCCACGGC
CTCCTGCAGCCCGTCCCCCAGCTCGGGCTCTGAGGGGCCAGGCCAGGTGG
ACAGTGGGCGGGGCTCAGACACCGAGGCCTCCGAGGGGGCGGAAGGGCTG
GGCGGCACCGACCTGCGGGGCCGGACCTGGGCCACTGCCCTAGCACTCGC
CTGGCTGGAGCACCGATGCGCCGCTGCCTTCGACGAGTGGGAACTGACAG
CGGCCAAGGCTGATTGCTGGCTGCGGGCCCAGCACTTGCCTGACGGCCTT
GACCTGGCCGCCCTCAAGGCCGCAGCCCGAGGGCTCTTCCTGCTACTGCG
CCACTGGGACCAAAACCTGCAGCTACACCTGCTGTGCTACAGCCCAGCGA
ACGTGTGAAGGCTGCCCCCTGCTGCTTGGGCTGGCGCCCCACCCAACACA
CTCAAGTCACTGCCGCCCAGGGCTGGCCTCTTGGTGCTCGGAAAGTGTAG
GCTGGTTCCAGCCTGTCCCCCACTGCTTCTTACTCCCTCCCTAGAGCCCT
CTTGCCCCCACAAAAAGTGCCTGCCTGTGCTCTCTCCCTCTCCTCCCACC
CCACTCACACTCCCCTCCATCCTTTGAGCTCCCTGCAACACAGTGGAAGG
GTAGAGAGCCACAGTCCCCAAATCCTATGCAATAAAGTGCCTCTTAGGGA
AAAA

The NOV3 nucleic acid was identified on chromosome 3 and has 2669 of 2799 bases (95%) identical to a gb:GENBANK-ID:AB047829|acc:AB047829.1 mRNA from Macaca fascicularis (Macaca fascicularis brain cDNA, clone:QccE-10361) (E=0.0).

A disclosed NOV3 polypeptide (SEQ ID NO:8) encoded by SEQ ID NO:7 is 1017 amino acid residues and is presented using the one-letter code in Table 3B. Signal P, Psort and/or Hydropathy results predict that NOV3 contains a signal peptide and is likely to be localized to the nucleus with a certainty of 0.6000. The most likely cleavage site is between amino acids 18 and 19: SHA-LR.

TABLE 3B
Encoded NOV3 protein sequence (SEQ ID NO:8)

MLVTGPCLLAGLESPSHALRADAPPHASSAATICVTLAEGHHCDRALEIL
LHPSEPHQPHLMLEGGSLSSAEYEARVRARRDFQRLQRGDSDGDRQVWFL
QRRFHKDILLNPVLALSFCPDLSSKPGHLGTATRELLFLLDSSSVAHKDA
IVLAVKSLPPQTLINLAVFGTLVQPLFPESRPCSDDAVQLICESIETLQV
PSGPPDVLAALDWAVGQPQHRAYPRQLFLLTAASPMAATTHRTLELMRWH
RGTARCFSFGLGPTCHQLLQGLSALSSRGRATALQPGRQSKPQLVQALRK
ALEPALSDISVDWFVPDTVEALLTPREIPALYPGDQLLGYCSLFRVDGFR
SRPPGGQEPGWQSSGGSVFPSPEEAPSAASPGTEPTGTSEPLGTGTVSAE
LSSPWAARDSEQSGTDALTDPVTDPGPNPSDTAIWRRIFQSSYIREQYVL
THCSASPEPGPGSTGSSESPGSQGPGSPEGSAPLEPPSQQGCRSLAWGEP
AGSRSCPLPAPTPAPFKVGALSTEVLGRQHRAALAGRSLSSPPGRANQVP
GRPRKPSLGATLDGPSPEPGQQLGQGLDDSGNLLSPAPMDWDMLMEPPEL
FTAVPPSGELAPPAVPPQAPRCHVVIRGLCGEQPMCWEVGVGLETLWGPG
DGSQPPSPPXTREAWDQALHRLTAASVVRLNEQLALRGGAETTADRGHAR
RCWLRALQTSKVSSAPSCFTCPVAVDATTREVLPGAALQVCSSEPAEPPG
TPPASHSHLDAAPLPTVYSKGAWDSDQNGNSKRALGDPATPTEGPRRPPP
RPPCRLSMGRRIKICSPDPGQANNSEGSDHDYLPLVRLQEAPGSFRLDAP
FCAAVRISQERLCRASPFAVHRASLSPTSASLPWALLGPGVGQGDSATAS
CSPSPSSGSEGPGQVDSGRGSDTEASEGAEGLGGTDLRGRTWATAVALAW
LEHRCAAAFDEWELTAAKADCWLRAQHLPDGLDLAALKAAARGLFLLLRH
WDQNLQLHLLCYSPANV

The NOV3 amino acid sequence has 767 of 803 amino acid residues (95%) identical to, and 779 of 803 amino acid residues (97%) similar to, the 803 amino acid residue ptnr:TREMBLNEW-ACC:BAB12255 protein from Macaca fascicularis (Crab eating macaque) (Cynomolgus monkey) (Hypothetical 84.7 Kda Protein) (E=0.0).

NOV3 is expressed in at least the following tissues: lung. This information was derived by determining the tissue sources of the sequences that were included in the invention including but not limited to SeqCalling sources, Public EST sources, genomic clone sources, literature sources, and/or RACE sources. The NOV3 sequence is also predicted to be expressed in the following tissues because of the expression pattern of (GENBANK-ID: gb:GENBANK-ID:AB047829|acc:AB047829.1) a closely related Macaca fascicularis brain cDNA, clone:QccE-10361 homolog in species Macaca fascicularis: brain.

Possible small nucleotide polymorphisms (SNPs) found for NOV3 are listed in Table 3C.

TABLE 3C
SNPs

| | | | Amino | |
|---|---|---|---|---|
| | Nucleotide | Base | Acid | Base |
| Variant | Position | Change | Position | Change |
| 13377063 | 3196 | A > G | Silent | N/A |

NOV3 has homology to the amino acid sequences shown in the BLASTP data listed in Table 3D.

TABLE 3D
BLAST results for NOV3

| Gene Index/ | | Length | Identity | Positives | |
|---|---|---|---|---|---|
| Identifier | Protein/Organism | (aa) | (%) | (%) | Expect |
| gi\|12654717\|gb\|AAH01200.1\| | Unknown (protein | 1001 | 826/972 | 831/972 | 0.0 |
| AAH01200 | for | | (84%) | (84%) | |
| (BC001200) | IMAGE: 3356192) | | | | |
| | [Homo sapiens] | | | | |
| gi\|9967105\|dbj\|BAB12255.1\| | hypothetical | 803 | 646/804 | 657/804 | 0.0 |
| (AB047829) | protein [Macaca | | (80%) | (81%) | |
| | fascicularis] | | | | |
| gi\|15295425\|ref\|XP_029078.2\| | similar to | 620 | 494/611 | 494/611 | 0.0 |
| (XM_029078) | Unknown (protein | | (80%) | (80%) | |
| | for | | | | |
| | IMAGE: 3356192) | | | | |
| | [Homo sapiens] | | | | |
| gi\|11231113\|dbj\|BAB18151.1\| | hypothetical | 260 | 192/247 | 194/247 | 5e−97 |
| (AB051125) | protein [Macaca | | (77%) | (77%) | |
| | fascicularis] | | | | |
| gi\|12855146\|dbj\|BAB30227.1\| | data source: SPTR, | 1209 | 142/346 | 229/346 | 2e−72 |
| (AK016425) | source | | (41%) | (66%) | |
| | key: 075668, | | | | |
| | evidence: ISS˜homolog | | | | |
| | to DJ745E8.1 | | | | |
| | (BREAST CANCER | | | | |
| | SUPPRESSOR | | | | |
| | CANDIDATE 1 | | | | |
| | (BCSC-1) LIKE) | | | | |
| | (FRAGMENT)˜putative | | | | |
| | [Mus musculus | | | | |

Table 3F list the domain description from DOMAIN analysis results against NOV3. This indicates that the NOV3 sequence has properties similar to those of other proteins known to contain these domains.

TABLE 3F

Domain Analysis of NOV3
gnl|Smart|smart00327, VWA, von Willebrand factor (vWF) type A domain;
VWA domains in extracellular eukaryotic proteins mediate adhesion via
metal ion-dependent adhesion sites (MIDAS). Intracellular VWA domains
and homologues in prokaryotes have recently been identified. The
proposed VWA domains in integrin beta subunits have recently been
substantiated using sequence-based methods (Pointing et al. Adv Prot
Chem (2000) in press). (SEQ ID NO:68)
CD-Length = 180 residues, 97.2% aligned
Score = 42.0 bits (97), Expect = 2e−04

| | |
|---|---|
| Query: 134 | RELLFLLDSS-SVAH------KDAIVLAVKSL---PPQTLINLAVFGTLVQPLFPESRPC 183 |
| | +++\|\|\|\| \| \|+ \|+ ++ \|+ \| \| + \| \| + + \|\|\| + |
| Sbjct: 2 | LDVVFLLDGSGSMGGNRFELAKEFVLKLVEQLDIGPDGDRVGLVTFSSDARVLFPLNDSQ 61 |
| Query: 184 | SDDAVQLICESIETLQV-PSGPPDVLAALDWAVGQPQH------RAYPRQLFLLTAASPM 236 |
| | \| \|\|+ \|++ +\| \| ++ \|\|\|++\|+ \| \|+ \| \|+\| |
| Sbjct: 62 | SKDALL---EALASLSYSLGGGTALGAALEYALENLFSESAGSRRGAPKVLILITDGESN 118 |
| Query: 237 | AATTHRTLELMRWHRGTARCFSFGLGPTCHQLLQGLSALSSRGPATALQPGRQSKPQLVQ 296 |
| | \| + \| \|+\| + \| \|+\| + \| \|+ |
| Sbjct: 119 | DGGEDILKAAKELKRSGVKVFVVGVGN--DVDEEELKKLASAPCGVFVVEDLPSLLDLLI 176 |

NOV3 described herein bears resemblance to BCSC-1 a molecule that is a candidate for suppression of breast cancer and this molecule may play a similar role in the lung and brain.

The NOV3 nucleic acid of the invention encoding a BCSC-1-like protein includes the nucleic acid whose sequence is provided in Table 3A, or a fragment thereof. The invention also includes a mutant or variant nucleic acid any of whose bases may be changed from the corresponding base shown in Table 3A while still encoding a protein that maintains its BCSC-1-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 5% of the residues may be so changed.

The NOV3 protein of the invention includes the BCSC-1-like protein whose sequence is provided in Table 3B. The invention also includes a mutant or variant protein any of whose residues may be changed from the corresponding residue shown in Table 3B while still encoding a protein that maintains its BCSC-1-like activities and physiological functions, or a functional fragment thereof. In the mutant or variant protein, up to about 5% of the bases may be so changed.

The NOV3 nucleic acids and proteins of the invention are useful in potential diagnostic and therapeutic applications implicated in various diseases and disorders described below and/or other pathologies. For example, the compositions of the present invention will have efficacy for treatment of patients suffering from: cancer, trauma, regeneration (in vitro and in vivo), viral/bacterial/parasitic infections, systemic lupus erythematosus, autoimmune disease, asthma, emphysema, scleroderma, allergy, ARDS and other diseases, disorders and conditions of the like.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV4

NOV4 includes five novel Lymphocyte antigen precursor-like or Lymphocyte antigen LY-6F-like proteins disclosed below. The disclosed proteins have been named NOV4a-NOV4e.

NOV4a

A disclosed NOV4a nucleic acid of 468 nucleotides (also referred to as CG56417-01) encoding a novel Lymphocyte antigen precursor-like protein is shown in Table 4A. An open reading frame was identified beginning with an ATG initiation codon at nucleotides 31-33 and ending with a TGA codon at nucleotides 439-441. Putative untranslated regions, if any, upstream from the initiation codon and downstream from the termination codon are underlined in Table 4A, and the start and stop codons are in bold letters.

TABLE 4A
NOV4a nucleotide sequence (SEQ ID NO:9).

TGAAGTTTGTCTGTGCACTAGCACCCTGGAATGAGCAGTCTCCAGGCCAT
GAAGACCTTGTCCCTGGTCCTGCTGGTGGCCCTGCTGAGCATGGAGAGAG
CTCAGGGTCTGCGCTGCTACAGATGCTTGGCGGTCTTGGAAGGGGCCTCC
TGCAGCGTGGTCTCGTGCCCCTTCCTGGATGGGGTCTGTGTCTCCCAGAA
AGTGAGCTTAAGTCTGAGCAAGAAAAGAAGAAAAGAAAAAAACAAGCTCT
CCCTCCTCTCCTGCCAGAAGGACGTCGGATTCCCCCTGCTGAAACTTACA
AGTGCCGTTGTGGACTCCCAGATCTCTTGCTGCAAGGCAGACCTCTGCAA
TGCGGTGGTCCTGGCAGCCAGCAGCCCCTGGGCCCTGTGCGTACAGCTCC
TGCTCAGCCTGGGGTCAGTCTTCCTCTGGGCCCTGCTGTGAGGGCCCTTT
CCCGCCCTCTCCCCCGCA

A NOV4a polypeptide (SEQ ID NO:10) encoded by SEQ ID NO:9 has 136 amino acid residues and is presented using the one-letter code in Table 4B.

TABLE 4B
Encoded NOV4a protein sequence (SEQ ID NO:10).

MSSLQAMKTLSLVLLVALLSMERAQGLRCYRCLAVLEGASCSVVSCPFLD
CVCVSQKVSLSLSKKRRKEKNKLSLLSCQKDVGFPLLKLTSAVVDSQISC
CKGDLCNAVVLAASSPWALCVQLLLSLGSVFLWALL

NOV4b

A disclosed NOV4b nucleic acid of 610 nucleotides (also referred to as CG56417-02) encoding a novel Lymphocyte antigen precursor-like protein is shown in Table 4C. An open reading frame was identified beginning with an ATG initiation codon at nucleotides 47-49 and ending with a TAG codon at nucleotides 515-517. Putative untranslated regions, if any, upstream from the initiation codon and downstream from the termination codon are underlined in Table 4C, and the start and stop codons are in bold letters.

TABLE 4C
NOV4b nucleotide sequence (SEQ ID NO:11).

CTTGTAAGGGCGAGACAGCAGAGACTGGCACCAGGGAGGCTCCTCCATGG
GAGTCTTCCATGATTACTCACAGCGGGTGGGCAGAGGTGTTGCTAGGAAG
CATGTTCTGGGGGGGTCCTCTGGGTGCACACGTGCAGTAGCTGCACCTGC
TTGCTCATACGTCGCATGTGTCATTAGCATCTTAAATCTCCACCAGGGGT
GTGTTTTCTTGCCCTCTCTCCCAGCTCAGGGTCTGCGCTGCTACAGATGC
TTGGCGGTCTTGGAAGGGGCCTCCTGCAGCGTGGTCTCGTGCCCCTTCCT
GGATGGGGTCTGTGTCTCCCAGAAAGTGAGCGTTTTGGCAGTGAGTCCCT
GGGGTGCCAGGGCAGAGGGCAGGTTAAGTGCCCTTGTGGACTCCCAGATC
TCTTGCTGCAAGGGAGACCTCTGCAATGCGGTGGTCCTGGCAGCCGGCAG
CCCCTGGGCCCTGTGCGTACAGCTCCTGCTCAGCCTGGGGTCAGTCTTCC
TCTGGGCCCTGCTGTGAGGGCCTTTCCCGCCCTCTCCCCCGCAGGCCTAC
CCTCTGTCCCTGTGCGTCACCAGCTGCTTGGTTTTGAAGAGCTGCCTCAC
TGAATTGCAG

The disclosed NOV4b nucleic acid sequence, localized to chromsome 8, has 192 of 249 bases (77%) identical to a gb:GENBANK-ID:HSU85047|acc:U85047.1 mRNA from Homo sapiens (Human FRA3B region corresponding to the breakpoint junction in lung tumor cell line NCIH211) (E=1.1e⁻²²).

A NOV4b polypeptide (SEQ ID NO:12) encoded by SEQ ID NO:11 has 156 amino acid residues and is presented using the one-letter code in Table 4D. Signal P, Psort and/or Hydropathy results predict that NOV4b does not contain a signal peptide and is likely to be localized to the plasma membrane with a certainty of 0.6000.

TABLE 4D
Encoded NOV4b protein sequence (SEQ ID NO:12).

MGVFHDYSQRVGRGVARKHVLGGSSGCTRAVAAPACSYVACVISILNLHQ
GCVFLPSLPAQGLRCYRCLAVLEGASCSVVSCPFLDGVCVSQKVSVLAVS
PWGARAEGRLSAVVDSQISCCKGDLCNAVVLAAGSPWALCVQLLLSLGSV
FLWALL

NOV4c

A disclosed NOV4c nucleic acid of 406 nucleotides (also referred to as CG56417-03) encoding a novel Lymphocyte antigen precursor-like protein is shown in Table 4E. An open reading frame was identified beginning with an ATG initiation codon at nucleotides 2-4 and ending with a TGA codon at nucleotides 404-406. Putative untranslated regions, if any, upstream from the initiation codon and downstream from the termination codon are underlined in Table 4E, and the start and stop codons are in bold letters.

TABLE 4E
NOV4c nucleotide sequence (SEQ ID NO:13).

AATGAGCAGTCTCCAGGCCATGAAGACCTTGTCCCTGGTCCTGCTGGTGG
CCCTGCTGAGCATGGAGAGAGCTCAGGGTCTGCGCTGCTACAGATGCTTG
GCGGTCTTGGAAGGGGCCTCCTGCAGCGTGGTCTCGTGCCCCTTCCTGGA
TGGGGTCTGTGTCTCCCAGAAAGTGAGCGTCTTTGGCAGTAAAGTGAGAG
GGGAGAACAAGCTCTCCCTCCTCTCCTGCCAGAAGGACGTCGGATTCCCC
CTGCTGAAACTTACAAGTGCCGTTGTGGACTCCCAGATCTCTTGCTGCAA
GGGAGACCTCTGCAATGCGGTGGTCCTGGCAGCCGGCAGCCCCCGGGCCC
TGTGCGTACAGCTCCTGCTCAGCCTGGGGTCAGTCTTCCTCTGGGCCCTG
CTGTGA

The disclosed NOV4c nucleic acid sequence, localized to chromsome 8, has 260 of 403 bases (64%) identical to a gb:GENBANK-ID:RATLY6CA|acc:M30691.1 mRNA from Rattus norvegicus (Rat Ly6-C antigen mRNA, exon 2) (E=1.2e⁻¹⁶).

A NOV4c polypeptide (SEQ ID NO:14) encoded by SEQ ID NO:13 has 134 amino acid residues and is presented using the one-letter code in Table 4F. Signal P, Psort and/or Hydropathy results predict that NOV4c contains a signal peptide and is likely to be localized to the plasma membrane with a certainty of 0.9190. The most likely cleavage site for a NOV4c polypeptide is between amino acids 26 and 27: AQG-LR

TABLE 4F
Encoded NOV4c protein sequence (SEQ ID NO:14).

MSSLQANKTLSLVLLVALLSMERAQGLRCYRCLAVLEGASCSVVSCPFLD
GVCVSQKVSVTGSKVRGENKLSLLSCQKDVGFPLLKLTSAVVDSQISCCK
GDLCNAVVLAAGSPRALCVQLLLSLGSVFLWALL

The NOV4c amino acid sequence has 61 of 134 amino acid residues (45%) identical to, and 79 of 134 amino acid residues (58%) similar to, the 134 amino acid residue ptnr:SPTREMBL-ACC:Q63318 protein from Rattus norvegicus (Rat) (RAT LY6-C ANTIGEN) (E=4.4e⁻²²).

NOV4d

A disclosed NOV4d nucleic acid of 611 nucleotides (also referred to as CG56417-04) encoding a novel Lymphocyte antigen LY-6F-like protein is shown in Table 4G. An open reading frame was identified beginning with an ATG initiation codon at nucleotides 47-49 and ending with a TGA codon at nucleotides 602-604. Putative untranslated regions, if any, upstream from the initiation codon and downstream from the termination codon are underlined in Table 4G, and the start and stop codons are in bold letters.

TABLE 4G
NOV4d nucleotide sequence (SEQ ID NO:15).

CTTGTAAGGGCGAGACAGCAGAGACTGGCACCAGGGAGGCTCCTCCATGG
GAGTCTTCCATGATTACTCACAGCGGGTGGGCAGAGGTGTTGCTAGGAAG
CATGTTCTGGGGGGGTCCTCTGGGTGCACACGTGCAGTAGCTGCACCTGC
TTGCTCATACGTCGCATGTGTCATTAGCATCTTAAATCTCCACCAGGGGT
GTGTTTTCTTGCCCTCTCTCCCAGCTCAGGGTCTGCGCTGCTACAGATGC
TTGGCGGTCTTGGAAGGGGCCTCCTGCAGCGTGGTCTCGTGCCCCTTCCT
GCATGGGGTCTGTGTCTCCCAGAAAGTGAGCGTCTTTGGCAGTGAGTCCC
TGGGGTGCCAGGGCAGAGGGCAGGTTAAGTGCCGTTGTGGACTCCCAGAT
CTCTTGCTGCAAAGGGAGACCTCTGCAATGCGGTGGTCCTGGCAGCCGGC
AGCCCCTGGGCCCTGTGCGTACAGCTCCTGCTCAGCCTGGGGTCAGTCTT
CCTCTGGGCCCTGCTGTGAGGGCCTTTCCCGCCCTCTCCCCCGCAGGCCT
ACCCTCTGTCCCTGTGCGTCACCAGCTGCTTGGTTTTGAAGAGCTGCCTC
ACTGAATTGCAG

The disclosed NOV4d nucleic acid sequence, localized to chromsome 8, has 198 of 256 bases (77%) identical to a gb:GENBANK-ID:AP000509|acc:AP000509.1 mRNA from Homo sapiens (Homo sapiens genomic DNA, chromosome 6p21.3, HLA Class I region, section 8/20) (E=5.4e ⁻²³).

A NOV4d polypeptide (SEQ ID NO:16) encoded by SEQ ID NO:15 has 185 amino acid residues and is presented using the one-letter code in Table 4H. Signal P, Psort and/or Hydropathy results predict that NOV4d does not contain a signal peptide and is likely to be localized to the plasma membrane with a certainty of 0.7900.

TABLE 4H
Encoded NOV4d protein sequence (SEQ ID NO:16).

MGVFHDYSQRVGRGVARKHVLGGSSGCTRAVAAPACSYVACVISILNLHQ
GCVFLPSLPAQGLRCYRCLAVLEGASCSVVSCPFLDGVCVSQKVSVFGSE
SLGCQGRGQVKCRCGLPDLLLQGRPLQCGGPGSRQPLGPVRTAPAQPGVS
LPLGPAVRAFPALSPAGLPSVPVRHQLLGFEELPH

The NOV4d amino acid sequence has 24 of 57 amino acid residues (42%) identical to, and 35 of 57 amino acid residues (61%) similar to, the 134 amino acid residue ptnr:SWISSNEW-ACC:P35460 protein from Mus musculus (Mouse) (Lymphocyte Antigen LY-6F.1 Precursor) (E=2.4e⁻⁰⁷).

The disclosed NOV4d is expressed in at least the following tissues: Brain, Pituitary Gland, Placenta, Lung, Trachea, Kidney, Colon and Whole Organism. This information was derived by determining the tissue sources of the sequences that were included in the invention including but not limited to SeqCalling sources, Public EST sources, Literature sources, and/or RACE sources.

NOV4e

A disclosed NOV4e nucleic acid assembly (also referred to as 172885384) of NOV4d (CG56417-04) encoding a novel Lymphocyte antigen LY-6F-like protein is shown in Table 4I. The cDNA coding for the mature form of the full length NOV4d from residue 49 to 183 was targeted for “in-frame” cloning by PCR. The insert 172885384 was found to encode an open reading frame between residues 49 and 183 of the target sequence of NOV4d.

TABLE 4I
NOV4e nucleotide sequence (SEQ ID NO:17).

GGATCCCACCAGGGGTGTGTTTTCTTGCCCTCTCTCCCAGCTCACGGTCT
GCGCTGCTACAGATGCTTGGCGGTCTTGGAAGGGGCCTCCTGCAGCGTGG
TCTCGTGCCCCTTCCTGGATGGGGTCTGTGTCTCCCAGAAAGTGACCGTC
TTTGGCAGTGAGTCCCTOGGGTGCCAGGGCAGAGGGCAGGTTAAGTGCCG
TTGTGGACTCCCAGATCTCTTGCTGCAAGGGAGACCTCTGCAATGCGGTG
GTCCTGGCAGCCGGCAGCCCCTGGGCCCTGTGCGTACAGCTCCTGCTCAG
CCTGGGGTCAGTCTTCCTCTGGGCCCTGCTGTGAGGGCCTTTCCCGCCCT
CTCCCCCGCAGGCCTACCCTCTGTCCCTGTGCCTCACCAGCTGCTTGGTT
TTGAAGAGCTGCCTCACCTCGAG

A NOV4e polypeptide (SEQ ID NO:18) encoded by SEQ ID NO:17 has 136 amino acid residues an is presented using the one-letter code in Table 4J.

TABLE 4J
Encoded NOV4e protein sequence (SEQ ID NO:18).

GSHQGCVFLPSLPAQGLRCYRCLAVLEGASCSVVSCPFLDGVCVSQKVSV
FGSESLGCQGRGQVKCRCGLPDLLLQGRPLQCOGPGSRQPLGPVRTAPAQ
PGVSLPLGPAXJRAFPALSPAGLPSVPVRHQLLGFEELPHLE

Possible SNPs found for NOV4a are listed in Table 4K and possible SNPs for NOV4d are listed in Table 4L.

TABLE 4K
SNPs

| | | | Amino | |
|---|---|---|---|---|
| | Nucleotide | Base | Acid | Base |
| Variant | Position | Change | Position | Change |
| 13377070 | 53 | A > G | 8 | Lys > Arg |
| 13377074 | 161 | T > C | 44 | Val > Ala |
| 13377075 | 166 | T > C | 46 | Cys > Arg |
| 13377076 | 194 | C > A | 55 | Ser > Tyr |
| 13377077 | 196 | C > T | 56 | Gln > End |
| 13377078 | 300 | A > G | Silent | N/A |
| 13377068 | 362 | T > C | 111 | Leu > Pro |
| 13377079 | 370 | A > G | 114 | Ser > Gly |
| 13377067 | 371 | G > A | 114 | Ser > Asn |
| 13377080 | 431 | C > T | 134 | Ala > Val |
| 13377081 | 468 | A > G | Silent | N/A |

TABLE 4L
SNPs

| Consensus | | Base | |
|---|---|---|---|
| Position | Depth | Change | PAF |
| 61 | 31 | G > — | 0.065 |
| 79 | 32 | A > G | 0.062 |
| 89 | 33 | A > G | 0.061 |
| 105 | 48 | G > A | 0.042 |
| 166 | 51 | C > T | 0.157 |
| 174 | 51 | A > G | 0.039 |
| 192 | 51 | A > C | 0.039 |
| 207 | 51 | C > T | 0.039 |
| 236 | 51 | T > C | 0.059 |
| 237 | 51 | A > G | 0.039 |
| 265 | 52 | T > A | 0.038 |
| 294 | 49 | G > T | 0.327 |
| 322 | 49 | A > G | 0.041 |
| 327 | 49 | A > G | 0.061 |
| 360 | 48 | A > C | 0.042 |
| 373 | 48 | A > G | 0.042 |
| 419 | 47 | G > A | 0.043 |
| 448 | 47 | G > A | 0.064 |

NOV4a-NOV4e shares high degree of homology. Homologies to any of the above NOV4 protein will be shared by the other NOV4 proteins insofar as they are homologous to each other as shown above. Any reference to NOV4 is assumed to refer to the NOV4 proteins in general, unless otherwise noted.

NOV4a has homology to the amino acid sequences shown in the BLASTP data listed in Table 4N.

TABLE 4N
BLAST results for NOV4a

| Gene Index/ | Protein/ | Length | Identity | Positives | |
|---|---|---|---|---|---|
| Identifier | Organism | (aa) | (%) | (%) | Expect |
| gi\|17505223\|ref\|NP_510968.1\| | lysosomal | 434 | 405/483 | 409/483 | 0.0 |
| (NM_078483) | amino acid | | (83%) | (83%) | |
| | transporter 1 | | | | |
| | [Homo | | | | |
| | sapiens] | | | | |
| gi\|18426842\|ref\|NP_569099.1\| | lysosomal | 475 | 371/484 | 406/484 | 0.0 |
| (NM_130415) | amino acid | | (76%) | (83%) | |
| | transporter 1 | | | | |
| | [Rattus | | | | |
| | norvegicus] | | | | |
| gi\|17473038\|ref\|XP_058449.1\| | similar to | 504 | 231/514 | 307/514 | e−101 |
| (XM_058449) | lysosomal | | (44%) | (58%) | |
| | amino acid | | | | |
| | transporter 1 | | | | |
| | [Homo | | | | |
| | sapiens] | | | | |
| gi\|17449820\|ref\|XP_059717.1\| | similar to | 165 | 147/162 | 147/162 | 6e−76 |
| (XM_059717) | lysosomal | | (90%) | (90%) | |
| | amino acid | | | | |
| | transporter 1 | | | | |
| | (H. sapiens) | | | | |
| | [Homo | | | | |
| | sapiens] | | | | |
| gi\|18467570\|ref\|XP_079250.1\| | CG13384 gene | 504 | 169/433 | 234/433 | 9e−69 |
| (XM_079250) | product | | (39%) | (54%) | |
| | [Drosophila | | | | |
| | melanogaster] | | | | |

Table 4P lists the domain description from DOMAIN analysis results against NOV4a. This indicates that the NOV4a sequence has properties similar to those of other proteins known to contain these domains.

TABLE 4P

Domain Analysis of NOV4a
gnl|Pfam|pfam01490, Aa_trans, Transmembrane Lymphocyte antigen
precursor protein. This transmembrane region is found in many
Lymphocyte antigen precursors including UNC-47 and MTR. UNC-47 encodes
a vesicular amino butyric acid (GABA) transporter, (VGAT). UNC-47 is
predicted to have 10 transmembrane domains . MTR is a N system
Lymphocyte antigen precursor system protein involved in
methyltryptophan resistance. Other members of this family include
proline transporters and amino acid permeases. (SEQ ID NO:69)
CD-Length = 370 residues, 85.1% aligned
Score = 119 bits (299), Expect = 3e−28

| | |
|---|---|
| Query: 91 | AVKNAGIVMGPISLLIIGIVAVHCMGILVKCAHHFCRRLNKSFVDYGDTVMYGLESSPCS 150 |
| | \| \| \| + \| + \|\|+ \| + ++ +\| +\| + + \| \|++\| \| + \| \| |
| Sbjct: 1 | AFKQLGWIPGLVLLLLAGFITLYTGLLLSECYEYVPGKRNDSYLDLGRSAYGGKGLLLTS 60 |
| Query: 151 | WLRNHAHWGRRVVDFFLIVTQLGFCCVYFVPLADNFKQVIEAANGTTNNCHNNETVILTP 210 |
| | ++ \| \| \| + \| ++\| ++ \| +\| |
| Sbjct: 61 | FVG-------------QYVNLFGVNIGYLILAGDLLPKII------SSFCGDNCD----- 96 |
| Query: 211 | TMDSRLYNLSFLPFLVLLVFIPNLRALSI--FSLLANITMLVSLVMIYQFIVFRYNLSVF 268 |
| | +\| +++ \| ++ \| \|\| \| \|\|\| \| +++ \| + + \| + + |
| Sbjct: 97 | HLDGNSWIIIFAAIIITLSFIPNFNLLSISSLSAFSSLAYLSIISFLIIVAVIAGIFVLL 156 |
| Query: 269 | QRIPDPSHLPLVAPWKTYPLFFGTAIFSFEGIGMVLPLENKMKDPR--KFPLILYLGMVI 326 |
| | + \| \| \| +\|+\|\|\| ++\|\|++\| \|\| \| \|\| +\| + ++\| |
| Sbjct: 157 | GAVYGILWSPSETKLTGLFLAIGIIVFAFEGHAVLLPIQNTMKSPSAKKFKKVLNVAIII 216 |
| Query: 327 | VTILYISLGCLGYLQFGANIQGSITLNLP---LYQSVKLLYSIGIFFTYALQFYVPAEII 383 |
| | \|\|+\|\|\| +\| \|\|\| \|\| \|++\|+\| \|\|\|\| + \| \| + \| \|+ \|\| + \|\| |
| Sbjct: 217 | VTVLYILVGFFGYLTFGNNVKGNILLNLPNNPFWLIVNLNLVVAILLTFPLQAFPIVRII 276 |
| Query: 384 | IPFFV---SRAPEHCELVVDLFVRTVLVCLTILAILIPR 419 |
| | + \|\| +\|+ + ++\| ++\|\|\|+\| |
| Sbjct: 277 | ENLLTKKNNFAPNKSKLLRVVIRSGLVVFTLLIAILVPF 315 |

Murine Ly-6 molecules are a family of cell surface glycoproteins which have interesting patterns of tissue expression during haematopoiesis from multipotential stem cells to lineage committed precursor cells, and on specific leucocyte subpopulations in the peripheral lymphoid tissues. These interesting patterns of tissue expression suggest an intimate association between the regulation of Ly-6 expression and the development and homeostasis of the immune system. Ly-6 molecules are low molecular weight phosphatidyl inositol anchored glycoproteins with remarkable amino acid homology throughout a distinctive cysteine rich protein domain that is associated predominantly with O-linked carbohydrate. The in vivo functions of Ly-6 molecules are not known although in vitro studies suggest a role in cellular activation (Gumley et al., Immunol Cell Biol.; 73 (4):277-96, 1995).

The Ly-6 alloantigens have been shown to play a critical role in T lymphocyte activation. LeClair et al. (LeClair et al., EMBO J. 5 (12):3227-34, 1986) isolated a Ly-6 cDNA, synthetic oligonucleotides, based on the partial amino acid sequence of purified Ly-6E.1 protein, were used to probe a cDNA library. The synthetic oligonucleotides or the isolated cDNA detected a 1.1-kb RNA species. Sequence analysis of the cDNA clone revealed that the Ly-6E.1 protein consists of a 26-amino acid leader followed by a 108-residue, cysteine-rich, core protein with no N-linked glycosylation sites. Southern blot analysis of genomic DNAs revealed multiple bands indicating a family of related genes. Using recombinant inbred and Ly-6 congenic strains of mice, restriction fragment length polymorphisms were demonstrable, and correlated with the Ly-6 allotype of the DNA donors. This probe will enable further molecular genetic analysis of the role of Ly-6-linked proteins in the process of T lymphocyte activation. Isolation of Ly-6 genomic clones may promote a further understanding of the complex tissue-specific expression patterns characteristic of Ly-6-linked genes (LeClair et al., 1986).

NOV4 will have similar properties as other lymphocyte antigen precursor and Ly-6 proteins and may be involved in the regulation of T lymphocyte activation.

The NOV4 nucleic acid of the invention encoding a Lymphocyte antigen precursor-like protein includes the nucleic acid whose sequence is provided in Tables 4A, 4C, 4E, 4G and 4I or a fragment thereof. The invention also includes a mutant or variant nucleic acid any of whose bases may be changed from the corresponding base shown in Tables 4A, 4C, 4E, 4G and 4I while still encoding a protein that maintains its Lymphocyte antigen precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 23% of the NOV4b residues, about 36% of the NOV4c residues and about 23% of the NOV4d residues may be so changed.

The NOV4 protein of the invention includes the Lymphocyte antigen precursor-like protein whose sequence is provided in Tables 4B, 4D, 4F, 4H and 4J. The invention also includes a mutant or variant protein any of whose residues may be changed from the corresponding residue shown in Tables 4B, 4D, 4F, 4H and 4J while still encoding a protein that maintains its Lymphocyte antigen precursor-like activities and physiological functions, or a functional fragment thereof. In the mutant or variant protein, up to about 58% of the NOV4b bases, about 55% of the NOV4c bases and about 58% of the NOV4d bases may be so changed.

The NOV4 nucleic acids and proteins of the invention are useful in potential diagnostic and therapeutic applications implicated in various diseases and disorders described below and/or other pathologies. For example, the compositions of the present invention will have efficacy for treatment of patients suffering from: adrenoleukodystrophy, congenital adrenal hyperplasia, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, diabetes, autoimmune disease, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, growth and reproductive disorders, systemic lupus erythematosus, autoimmune disease, asthma, emphysema, scleroderma, allergy, ARDS and other diseases, disorders and conditions of the like.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV4 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV5

A disclosed NOV5 nucleic acid of 1260 nucleotides (also referred to as CG57220-04) encoding a novel TASK 4-like protein is shown in Table 5A. An open reading frame was identified beginning with an ATG initiation codon at nucleotides 107-109 and ending with a TAG codon at nucleotides 1163-1165. Putative untranslated regions upstream from the intitation codon and downstream from the termination codon are underlined in Table 5A, and the start and stop codons are in bold letters.

TABLE 5A
NOV5 Nucleotide Sequence (SEQ ID NO:19)

CTCAAGCAGGCGTTTGCGAGAGGAGATACGAGCTGGACGCCTGGCCCTTC
CCTCCCACCGGGTCCTAGTCCACCGCTCCCGGCGCCGGCTCCCCGCCTCT
CCCGCTATGTACCGACCGCGAGCCCGGGCGGCTCCCGAGGGCAGGGTCCG
GGGCTGCGCGGTGCCCGGCACCGTGCTCCTGCTGCTCGCCTACCTGGCTT
ACCTGGCGCTGGGCACCGGCGTGTTCTGGACGCTGGAGGGCCGCGCGGCG
CAGGACTCCAGCCGCAGCTTCCAGCGCGACAAGTGGGAGCTGTTGCAGAA
CTTCACGTGTCTGGACCGCCCGGCGCTGGACTCGCTGATCCGGGATGTCG
TCCAAGCATACAAAAACGGAGCCAGCCTCCTCAGCAACACCACCAGCATG
GGGCGCTGGGAGCTCGTGGGCTCCTTCTTCTTTTCTGTGTCCACCATCAC
CACCATTGGCTATGGCAACCTGAGCCCCAACACGATGGCTGCCCGCCTCT
TCTGCATCTTCTTTGCCCTTGTGGGGATCCCACTCAACCTCGTGGTGCTC
AACCGACTGGGGCATCTCATGCAGCAGGGAGTAAACCACTGGGCCAGCAG
GCTGGGGGGCACCTGGCAGGATCCTGACAAGGCGCGGTGGCTGGCGGGCT
CTGGCGCCCTCCTCTCGGGCCTCCTGCTCTTCCTGCTGCTGCCACCGCTG
CTCTTCTCCCACATGGAGGGCTGGAGCTACACAGAGGGCTTCTACTTCGC
CTTCATCACCCTCAGCACCGTGGGCTTCGGCGACTACGTGATTGGAATGA
ACCCCTCCCAGAGGTACCCACTGTGGTACAAGAACATGGTGTCCCTGTGG
ATCCTCTTTGGGATGGCATGGCTGGCCTTGATCATCAAACTCATCCTCTC
CCAGCTGGAGACAAACCCATCCTCTCCCAGCTGGACACAACAGGGAGGGT
ATGTTCCTGCTGCCACCACAGCTCTAAGGAAGACTTCAAGTCCCAAAGCT
GGAGACAGGGACCTGACCGGGAGCCAGAGTCCCACTCCCCACAGCAAGGA
TGCTATCCAGAGGGACCCATGGGAATCATACAGCATCTGGAACCTTCTGC
TCACGCTGCAGGCTGTGGCAAGGACAGCTAGTTATACTCCATTCTTTGGT
CGTCGTCCTCGGTAGCAAGACCCCTGATTTTAAGCTTTGCACATGTCCAC
CCAAACTAAAGACTACATTTTCCATCCACCCTAGAGGCTGGGTGCAGCTA
TATGATTAAT

The NOV5 nucleic acid was identified on chromosome 6 and has 508 of 827 bases (61%) identical to a gb:GENBANK-ID:AF006823|acc:AF006823.1 mRNA from Homo sapiens (Homo sapiens TWIK-related acid-sensitive K+ channel (TASK) mRNA, complete cds) (E=1.2e⁻²⁹).

A disclosed NOV5 polypeptide (SEQ ID NO:20) encoded by SEQ ID NO:19 is 352 amino acid residues and is presented using the one-letter code in Table 5B. Signal P, Psort and/or Hydropathy results predict that NOV5 contains a signal peptide and is likely to be localized to the extracellularly with a certainty of 0.6400. The most likely cleavage site for a NOV5 polypeptide is between amino acids 48 and 49: RAA-QD.

TABLE 5B
Encoded NOV5 protein sequence (SEQ ID NO:20)

MYRPRARAAPEGRVRGCAVPGTVLLLLAYLAYLALGTGVFWTLEGRAAQD
SSRSFQRDKWELLQNFTCLDRPALDSLIRDVVQAYKNGASLLSNTTSMGR
WELVGSFFFSVSTITTIGYGNLSPNTMAARLFCTFFALVGIPLNLVVLNR
LGHLMQQGVNHWASRLGGTWQDPDKARWLAGSGALLSGLLLFLLLPPLLF
SHMEGWSYTEGFYFAFITLSTVGFGDYVIGMNPSQRYPLWYKNMVSLWIL
FGMAWLALIIKLILSQLETNPSSPSWRQQGGYVPAATTALRKTSSPKAGD
RDLTGSQSPTPHSKDAIQRDPWESYSIWNLLLTLQAVARTASYTPFFGRR
PR

The NOV5 amino acid sequence has 340 of 352 amino acid residues (96%) identical to, and 342 of 352 amino acid residues (97%) similar to, the 343 amino acid residue ptnr: TREMBLNEW-ACC:AAK28551 protein from Homo sapiens (Human) (Potassium Channel TASK-4) (E=5.0e⁻¹⁸³).

NOV5 is expressed in at least the following tissues: liver, lung, pancreas, placenta, aorta and heart. This information was derived by determining the tissue sources of the sequences that were included in the invention including but not limited to SeqCalling sources, Public EST sources, genomic clone sources, literature sources, and/or RACE sources. The sequence is predicted to be expressed in the following tissues because of the expression pattern of (GENBANK-ID: gb:GENBANK-ID:AF006823|acc:AF006823.1) a closely related Homo sapiens TWIK-related acid-sensitive K+ channel (TASK) mRNA, complete cds homolog in species Homo sapiens: pancreas, placenta, brain, lung, prostate, heart, kidney, uterus, small intestine and colon.

NOV5 has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|13507377\|gb\|AAK28551.1\| | potassium channel | 343 | 305/352 | 307/352 | e−164 |
| AF339912_1 | TASK-4 [Homo | | (86%) | (86%) | |
| (AF339912) | sapiens] | | | | |
| gi\|17025230\|ref\|NP_113648.2\| | potassium | 332 | 233/269 | 233/269 | e−128 |
| (NM_031460) | channel, | | (86%) | (86%) | |
| | subfamily K, | | | | |
| | member 17; 2P | | | | |
| | domain potassium | | | | |
| | channel Talk-2; | | | | |
| | potassium channel | | | | |
| | TASK-4; potassium | | | | |
| | channel TALK-2 | | | | |
| | [Homo sapiens] | | | | |
| gi\|9988111\|emb\|CAC07335.1\| | dJ137F1.1 (novel | 229 | 193/229 | 193/229 | e−103 |
| (AL136087) | member of the | | (84%) | (84%) | |
| | potassium channel | | | | |
| | subfamily K) | | | | |
| | [Homo sapiens] | | | | |
| gi\|9988112\|emb\|CAC07336.1\| | dJ137F1.2 (novel | 294 | 87/226 | 136/226 | 1e−41 |
| (AL136087) | member of the | | (38%) | (59%) | |
| | potassium channel | | | | |
| | subfamily K) | | | | |
| | [Homo sapiens] | | | | |
| gi\|14149764\|ref\|NP_115491.1\| | pancreatic 2P | 309 | 87/226 | 136/226 | 1e−41 |
| (NM_032115) | domain potassium | | (38%) | (59%) | |
| | channel TALK-1; | | | | |
| | potassium family, | | | | |
| | subfamily K, | | | | |
| | member 16 [Homo | | | | |
| | sapiens] | | | | |

The NOV5 nucleic acid of the invention encoding a TASK 4-like protein includes the nucleic acid whose sequence is provided in Table 5A, or a fragment thereof. The invention also includes a mutant or variant nucleic acid any of whose bases may be changed from the corresponding base shown in Table 5A while still encoding a protein that maintains its TASK 4-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39% of the residues may be so changed.

The NOV5 protein of the invention includes the TASK 4-like protein whose sequence is provided in Table 5B. The invention also includes a mutant or variant protein any of whose residues may be changed from the corresponding residue shown in Table 5B while still encoding a protein that maintains its TASK 4-like activities and physiological functions, or a functional fragment thereof. In the mutant or variant protein, up to about 4% of the bases may be so changed.

The NOV5 nucleic acids and proteins of the invention are useful in potential diagnostic and therapeutic applications implicated in various diseases and disorders described below and/or other pathologies. For example, the compositions of the present invention will have efficacy for treatment of patients suffering from: cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, aneurysm, fibromuscular dysplasia, stroke, systemic lupus erythematosus, autoimmune disease, asthma, emphysema, allergy, ARDS, Von Hippel-Lindau (VHL) syndrome, cirrhosis, diabetes, pancreatitis, fertility, cancer, tissue degeneration, bacterial/viral/parasitic infections and other diseases, disorders and conditions of the like.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV6

A disclosed NOV6 nucleic acid of 627 nucleotides (also referred to as CG57458-01) encoding a novel Copper transporter-like protein is shown in Table 6A. An open reading frame was identified beginning with an ATG initiation codon at nucleotides 31-33 and ending with a TGA codon at nucleotides 598-600. Putative untranslated regions upstream from the intitation codon and downstream from the termination codon are underlined in Table 6A, and the start and stop codons are in bold letters.

TABLE 6A
NOV6 Nucleotide Sequence (SEQ ID NO:21)

TCTGCTGACTCTCAACTTTTCTTGGAAAAATGGATCATTCCCATCATACG
GGGATGAGCTGTATGGACTCCAACAGTACCATGCAACGTCCTCACCATCA
CCCACCACTTCAGCCTCACACTCCCGTGGTGGAGGAGACAGCAACATGAT
GATGATGATGCCTATGACCTTCTACTTTGGCTTTAAGAATGTGGAACTAC
TGTTTTCCAGTTTGTGATCAATACAGCTGGAGAAATGGCTGGAGCTTTTG
TGGCAGTGCTTTTGCTACATTCCATGCCTGTCCCATAGCCCAAGAGAGCC
TGCTGTGTAGTCACAAGTCAGCTTTTGCTACAATTCCATGCCTGTCCCAG
GACCAAATGGAACCATCCTTATGGAGACACACAAAACTGTTGGGCAGCAG
ATGCTGAGCTTTCCTCACCTCCTGCAACAGTGCTGCACATCATCCAGGTG
GTCATAAGCTACCTCCTCATGCTCATCTTCATGACCTACATGGGTACCTC
TGCATTGCAGTAACAGCAGGGGCCGGTACAAGATACTTCCTCTTCAGCTG
GAGAAGGCAGTGGTAGTGCACATCACAGAGTATTGCCATTGACGTCAAAC
TCTATGGCATGGCCTTATC

The NOV6 nucleic acid was identified on chromosome 3.

A disclosed NOV6 polypeptide (SEQ ID NO:22) encoded by SEQ ID NO:21 is 189 amino acid residues and is presented using the one-letter code in Table 6B. Signal P, Psort and/or Hydropathy results predict that NOV6 contains a signal peptide and is likely to be localized to the plasma membrane with a certainty of 0.6000. The most likely cleavage site for a NOV6 polypeptide is between amino acids 68 and 69: TAG-EM.

TABLE 6B
Encoded NOV6 protein sequence (SEQ ID NO:22)

MDHSHHTGMSCMDSNSTMQRPHHHPTTSASHSRGGGDSNMMMMMPMTFYF
GFKNVELLFSSLVINTAGEMAGAFVAVLLLQFHACPIAQESLLCKSQVSF
CYNSMPVPGPNGTILMETHKTVGQQMLSFPHLLQTVLHIIQVVISYLLML
IFMTYNGYLCIAVTAGAGTRYFLFSWKKAVVVDITEYCH

The NOV6 amino acid sequence has 166 of 190 amino acid residues (87%) identical to, and 170 of 190 amino acid residues (89%) similar to, the 190 amino acid residue ptnr:SWISSNEW-ACC:O15431 protein from Homo sapiens (Human) (High-Affinity Copper Uptake Protein 1 (HCTR1) (E=1.9e⁻⁸³).

NOV6 is expressed in at least the following tissues: brain. This information was derived by determining the tissue sources of the sequences that were included in the invention including but not limited to SeqCalling sources, Public EST sources, genomic clone sources, literature sources, and/or RACE sources.

NOV6 has homology to the amino acid sequences shown in the BLASTP data listed in Table 6C.

TABLE 6C
BLAST results for NOV6

| Gene Index/ | | Length | Identity | Positives | |
|---|---|---|---|---|---|
| Identifier | Protein/Organism | (aa) | (%) | (%) | Expect |
| gi\|4507015\|ref\|NP_001850.1\| | solute carrier | 190 | 149/191 | 153/191 | 2e−67 |
| (NM_001859) | family 31 (copper | | (78%) | (80%) | |
| | transporters), | | | | |
| | member 1; hCTR1; | | | | |
| | copper | | | | |
| | transporter 1 | | | | |
| | [Homo sapiens] | | | | |
| gi\|17940111\|gb\|AAL49494.1\| | high-affinity | 189 | 135/191 | 141/191 | 6e−59 |
| AF320815_1 | copper uptake | | (70%) | (73%) | |
| (AF320815) | protein [Sus | | | | |
| | scrofa] | | | | |
| gi\|12229733\|sp\|Q9JK41\| | High-affinity | 187 | 133/187 | 139/187 | 1e−58 |
| COP1_RAT | copper uptake | | (71%) | (74%) | |
| | protein 1 (rCTR1) | | | | |
| | (Copper | | | | |
| | transporter 1) | | | | |
| gi\|18652812\|gb\|AAL76092.1\| | high affinity | 188 | 113/189 | 130/189 | 3e−50 |
| (AY077715) | copper uptake | | (59%) | (67%) | |
| | protein [Danio | | | | |
| | rerio] | | | | |
| gi\|17462341\|ref\|XP_067707.1\| | similar to solute | 175 | 118/189 | 126/189 | 6e−50 |
| (XM_067707) | carrier family 31 | | (62%) | (66%) | |
| | (copper | | | | |
| | transporters), | | | | |
| | member 1; hCTR1; | | | | |
| | copper | | | | |
| | transporter 1 | | | | |
| | [Homo sapiens] | | | | |

Copper is an element essential for life, but excessive copper can be toxic or even lethal to the cell. Therefore, cells have developed sophisticated ways to maintain a critical copper balance, with the intake, export, and intracellular compartmentalization or buffering of copper strictly regulated. The two related genes ATP7A and ATP7B, responsible for the human diseases Menkes syndrome and Wilson disease (WND), respectively, are involved in copper export. In S. cerevisiae, three copper uptake genes CTR1, CTR2, and CTR3 have been identified.

In mammals, however, the molecular basis for copper uptake is unknown. Zhou and Gitschier (Proc Natl Acad Sci USA. 94 (14):7481-6, 1997) isolated a human cDNA encoding COPT1, which they called CTR1, by functional complementation of the yeast high-affinity copper uptake mutant ctr1. The deduced 190-amino acid human CTR1 protein is similar to yeast CTR1 and Arabidopsis COPT1, a copper transporter also isolated by functional complementation of yeast ctr1. All 3 predicted proteins have 3 transmembrane domains and an N terminus that is rich in methionine and serine residues; the N terminus of human CTR1 is also abundant in histidines. The authors proposed that human CTR1 is a high-affinity copper uptake gene because it can complement the yeast ctr1 mutation, it can rescue multiple defects in ctr1 yeast, its expression in ctr1 yeast increases the concentration of cellular copper, and its overexpression in yeast leads to a vulnerability to the toxicity of copper overload. Northern blot analysis detected 2 major CTR1 transcripts of approximately 2 kb and 5.5 kb and a less abundant transcript of about 8.5 kb in all human organs and tissues examined. Zhou and Gitschier (1997) found that the 3-prime untranslated region of the human CTR1 gene contains a CA repeat marker (D9S262) that had been previously mapped to 9q31-q32. By analysis of YAC clones, they showed that CTR1 and CTR2 (COPT2) which is also located in 9q31-q32, are not adjacent genes. The molecular mechanisms responsible for the cellular uptake of copper in mammalian cells are unknown. The isolation of a human gene involved in this process by complementation of the yeast high-affinity copper uptake mutant, ctr1 is described. Besides complementing ctr1 growth defect on nonfermentable media, the human gene also rescues iron transport and SOD1 defects in ctr1 yeast. Overexpression of the gene in yeast leads to vulnerability to the toxicity of copper overload. In addition, its expression in ctr1 yeast significantly increases the level of cellular copper, as demonstrated by atomic absorption. It is proposed that this gene is a candidate for high-affinity copper uptake in humans and by analogy have named it hCTR1. The hCTR1 and yeast CTR1 predicted transmembrane proteins are 29% identical, but the human protein is substantially smaller in both the extracellular metal-binding and intracellular domains. An additional human gene similar to hCTR1, here named hCTR2, was identified in a database search. Both hCTR1 and hCTR2 are expressed in all human tissues examined, and both genes are located in 9q31/32. These studies, together with the previously recognized functional and sequence similarity between the Menkes/Wilson copper export proteins and CCC2 in yeast, demonstrate that similar copper homeostatic mechanisms are used in these evolutionarily divergent organisms.

The NOV6 nucleic acid of the invention encoding a Copper transporter-like protein includes the nucleic acid whose sequence is provided in Table 6A, or a fragment thereof. The invention also includes a mutant or variant nucleic acid any of whose bases may be changed from the corresponding base shown in Table 6A while still encoding a protein that maintains its Copper transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 13% of the residues may be so changed.

The NOV6 protein of the invention includes the Copper transporter-like protein whose sequence is provided in Table 6B. The invention also includes a mutant or variant protein any of whose residues may be changed from the corresponding residue shown in Table 6B while still encoding a protein that maintains its Copper transporter-like activities and physiological functions, or a functional fragment thereof. In the mutant or variant protein, up to about 13% of the bases may be so changed.

The NOV6 nucleic acids and proteins of the invention are useful in potential diagnostic and therapeutic applications implicated in various diseases and disorders described below and/or other pathologies. For example, the compositions of the present invention will have efficacy for treatment of patients suffering from: Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection and other diseases, disorders and conditions of the like.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV7

A disclosed NOV7 nucleic acid of 1478 nucleotides (also referred to as CG57454-01) encoding a novel Cytokeratin-like protein is shown in Table 7A. An open reading frame was identified beginning with an ATG initiation codon at nucleotides 21-23 and ending with a TGA codon at nucleotides 1464-1466. Putative untranslated regions upstream from the intitation codon and downstream from the termination codon are underlined in Table 7A, and the start and stop codons are in bold letters.

TABLE 7A
NOV7 Nucleotide Sequence (SEQ ID NO:23)

ACTCCACTCCTGCCTCCACCATGTCCATCAGGGTGACCCAGAAGTCCTAC
AAGGTGTCCACCTCTGGCCCCCAGGCCTTTAGCAGCCGCTTTTACACGAA
TGGGCCTGGTGCCCACATCAGCTCTTCAAGCCTCTCCCGAGTGAGCAGCA
GCAGCTTCCGGGGTGGCCTGGGCAGAGGCTATGGTGGGGCCAGCGGCATA
GGAGGCATCACCACTGTCACGTTCAACCAGAGCCTGCTGAGCCCTGTTAA
CCTGGAGGTAGATCCCAATATCCAGGCCATGCACACCCAGGAGAAGGAGC
AGATCAAGACCCTCAACAACAAGTTTGCCTCCTTCATCGACAAGGTACGG
TTCCTGCAGCAGAAGAACAAGATGCTGGAGACCAAGTGGAGCCTCCTGCA
GCAGCAGAAGATGGCTCGGATCAACGTATTTGAGAGCTACATGAACAACC
TTAGGCGGCAGCTGGAGGCTCTGGGCCAGGAGAAGCTGAAGCTGGAGGCG
GAGCTTGGCAATATGCAGGGGCTGGTGGAGGACTTCAAGAACAAGTATGA
GGATGAGATCAATAAGCGTACAGAAACGGAGAATGAATTTGTCCTCATCA
AGAAGGACATGGATGAAGCTTACATGAACAAGGCAGAGCTGGAGTCTCGC
CTGGAAGGGCTGACTGACGAGATCAACTTCCTCAGGCAACTGCATGAAGA
GGAGATCCAGGAGCTGCAGTCCCAGATCTCGGGCACGTCTGCGGTGCTGT
CCATGGACAACAGCCTCTCCCTGGACATGGACAGCATCATCGCTGAGGTC
AAGGCACAGGAGGAGGAGATCGCCAACCGCAGCTGGGCTGAGGCTGAGAG
CATGTACCAGATCAAGTATGCAGAGCTGCAGACGCTGGCTGGCAAGCACG
GGGATGACCTGCGGTGTACAAAGACTAAGATCTCCGAGATGAACCGGAAC
ATCAGCCGGCTCCAGGCTGAGATTGAGGGCCTCAAAGGCCAGAGGGCTTC
CCTGGAGGCCCCCATCGCAGATACCGAGCAGCGTGGGGAGCTGGCCGTTA
AGGATGCCAGCGCCAAGCGGTCGGAGCTGGAGGCCGCCCTGCAGCGGGCC
ATGCAGGACATGGCGCAGCAGCTGCGTGAGTACCAGGAGCTGATGAACGT
CAAACTGGCCCTGGACATGGAGATCGCCACCTACAGGAAGCTGCTGGAGG
GCGAGGAGAGCGCCCGGCTGGAGTCTGGGATGCAGAACACGAGTATCCAT
AGGAAGACCACCAGCGGCTATGCAGGTGGTCTGAGTTCCGCCTATGGGGG
CCTCACAAGCCCTGGCCTCAGCTATGGCCTAAGCTCCAGCTTTGGCTCTG
TCGCCGGCTCCAGCTCATTCAGCCGCACCGGCTCCGCCAGGGCCATGGTT
GTGCAGAAGATTGAGACCCGCGATGGGAAGCTGGTATCCGAGTCCTCTGA
CGTCCTGCCCAAGTGAAGAGCTGCGGCA

The NOV7 nucleic acid was identified on chromosome 3 and has 1187 of 1344 bases (88%) identical to a gb:GENBANK-ID:AF213884S2|acc:AF224669.1 mRNA from Homo sapiens (Homo sapiens mannosidase, beta A, lysosomal (MANBA) gene, and ubiquitin-conjugating enzyme E2D 3 (UBE2D3) genes, complete cds) (E=7.3e⁻²²⁸).

A disclosed NOV7 polypeptide (SEQ ID NO:24) encoded by SEQ ID NO:23 is 481 amino acid residues and is presented using the one-letter code in Table 7B. Signal P, Psort and/or Hydropathy results predict that NOV7 contains a signal peptide and is likely to be localized to the plasma membrane with a certainty of 0.3600.

TABLE 7B
Encoded NOV7 protein sequence (SEQ ID NO:24)

MSIRVTQKSYKVSTSGPQAFSSRFYTNGPGAHISSSSLSRVSSSSFRGGL
GRGYGGASGIGGITTVTFNQSLLSPVNLEVDPNIQAMHTQEKEQIKTLNN
KFASFIDKVRFLQQKNKMLETKWSLLQQQKMARINVFESYMNNLRRQLEA
LGQEKLKLEAELGNMQGLVEDFKNKYEDEINKRTETENEFVLIKKDMDEA
YMNKAELESRLEGLTDEINFLRQLHEEEIQELQSQISGTSAXTLSMDNSL
SLDMDSIIAEVKAQEEIANRSWAEAESMYQIKYAELQTLAGKHGDDLRCT
KTKISENNRNISRLQAEIEGLKGQRASLEAPIADTEQRGELAVKDASAKR
SELEAALQPAKQDDAQQLREYQELMNVKLALDMEIATYRKLLEGEESARL
ESGMQNTSIHRKTTSGYAGGLSSAYGGLTSPGLSYGLSSSFGSVAGSSSF
SRTGSARAMVVQKIETRDGKLVSESSDVLPK

The NOV7 amino acid sequence has 424 of 482 amino acid residues (87%) identical to, and 448 of 482 amino acid residues (92%) similar to, the 482 amino acid residue ptnr:SWISSPROT-ACC:P05787 protein from Homo sapiens (Human) (Keratin, Type II Cytoskeletal 8 (Cytokeratin 8) (K8) (CK 8)) (E=4.9e⁻²¹³).

NOV7 is expressed in at least the following tissues: Heart, Kidney, Liver, Lung, Muscle, Nose, Ovary, Pancreas, Parathyroid, Placenta, Pooled, Prostate, Stomach, Synovial membrane, Testis, Thyroid, Tonsil and Uterus. This information was derived by determining the tissue sources of the sequences that were included in the invention including but not limited to SeqCalling sources, Public EST sources, genomic clone sources, literature sources, and/or RACE sources.

NOV7 has homology to the amino acid sequences shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|105815\|pir\|\|A34720 | keratin 8, type | 483 | 335/484 | 355/484 | e−145 |
| | II cytoskeletal | | (69%) | (73%) | |
| | [Homo sapiens] | | | | |
| gi\|2506774\|sp\|P05787\| | KERATIN, TYPE II | 483 | 335/484 | 354/484 | e−145 |
| K2C8_HUMAN | CYTOSKELETAL 8 | | (69%) | (72%) | |
| | (CYTOKERATIN 8) | | | | |
| | (K8) (CK 8) [Homo | | | | |
| | sapiens] | | | | |
| gi\|4504919\|ref\|NP_002264.1\| | keratin 8; | 483 | 335/484 | 354/484 | e−145 |
| (NM_002273) | Keratin-8 [Homo | | (69%) | (72%) | |
| | sapiens] | | | | |
| gi\|181400\|gb\|AAA35748.1\| | cytokeratin 8 | 483 | 333/484 | 354/484 | 1e−144 |
| (M34225) | [Homo sapiens] | | (68%) | (72%) | |
| gi\|87303\|pir\|\|JS0487 | cytokeratin 8 | 482 | 335/484 | 354/484 | 1e−143 |
| | (version 1) [Homo | | (69%) | (72%) | |
| | sapiens] | | | | |

Table 7E lists the domain description from DOMAIN analysis results against NOV7. This indicates that the NOV7 sequence has properties similar to those of other proteins known to contain these domains.

TABLE 7E
Domain Analysis of NOV7

gnl|Pfam|pfam00038, filament, Intermediate filament protein.
(SEQ ID NO:70)
CD-Length = 312 residues, 99.7% aligned
Score = 261 bits (668), Expect = 5e−71

| | |
|---|---|
| Query: 90 | QEKEQIKTLNNXFASFIDKVRFLQQKNKMLETKWSLLQQQKMARI----NVFESYMNNLR 145 |
| | \|\|\|\|++ \|\|++ \|\|+\|\|\|\|\|\|\|+\|+\|\| \|\| \| \|+\|++ + +++\|+ + \|\| |
| Sbjct: 1 | NEKEQMQNLNDRLASYIDKVRFLEQQNKELEVKIEELRQKQAPSVSRLYSLYETEIEELR 60 |
| Query: 146 | RQLEALGQEKLKLEAELGNMQGLVEDFKNKYEDEINKRTETENEPThIKKDMDEAYMNKA 205 |
| | \|\|++ \| \|+ +\|+ \|+ \|++ \|\|\|+\|\|\|\|\|\|\| \| \| \|\|+ \| ++\|\|+\|\|\| + + |
| Sbjct: 61 | RQIDQLTNERARLQLEIDNLREAAEDFRKKYEDEINLRQEAENDLVGLRKDLDEATLARV 120 |
| Query: 206 | ELESRLEGLTDEINFLRQLHEEEIQELQSQISGTSAVLSMDNSLSLDMDSIIAEVKAQEE 265 |
| | +\|\|+++\| \| +\|+ \|\|++\|\|\|\|++\|\|\|+\|\| \| \| + \|++\|\| \| |
| Sbjct: 121 | DLENKVESLQEELEFLKKNHEEEVKELQAQIQDTVNVEMDAARKLDLTK-ALREIRAQYE 179 |
| Query: 266 | EIANRSWAEAESDYQIKYAELQTLAGKHGDDLRCTKTKISEMNPNISRLQAEIEGLKGQR 325 |
| | \|\|\| ++ \|\|\| \|+ \| \|\|\|\| \| ++\|+ \|\| \| +\|+\|+ \| \| \|+ \|++ \|\| \| |
| Sbjct: 180 | EIAKKNRQEAEEWYKSKLEELQTAAARNGEALRSAKEEITELRRQIQSLEIELQSLKAQN 239 |
| Query: 326 | ASLEAPIADTEQRGELAVKDASAKRSELEAALQRAKQDMAQQLREYQELMNVKLALDMEI 385 |
| | \|\|\|\| +\|+ \|+\| \|\| ++ \| \|+\|\| \|\|+ +++\|\|+\|\|\|\|\|\|\|\|++\|\|\|\|\|\|+\|\| |
| Sbjct: 240 | ASLERQLAELEERYELELRQYQALISQLEEELQQLREEMARQLREYQELLDVKLALDIEI 299 |
| Query: 386 | ATYRKLLEGEES 397 |
| | \|\|\|\|\|\|\|\|\|\|\|\| |
| Sbjct: 300 | ATYRKLLEGEES 311 |

Keratin 8 is a type II keratin (Moll et al., 1982). Endo A is the mouse equivalent. Endo B, which is the equivalent of human keratin 18, a type I keratin, is coexpressed with Endo A; the 2 appear to be the first intermediate filament (IF) proteins expressed during murine development (Jackson et al., Differentiation. 17 (3):161-79, 1980). Yamamoto et al. (Mol Endocrinol. 4 (3):370-4, 1990) studied a full-length cDNA for cytokeratin 8 from placenta. They determined the distribution of cytokeratin 8 mRNA in various fetal tissues and in placentae of different gestational ages. Keratins 8 and 18 of simple epithelia differ from the keratins of stratified epithelium in tissue expression and regulation.

Using PCR to study DNAs from somatic cell hybrids, Waseem et al. (Genomics. 7 (2):188-94, 1990) located a single active gene for keratin 8 on chromosome 12. This chromosome contains several genes for type II keratins and also the gene for keratin 18, the type I keratin that is coexpressed with keratin 8. This location of both members of a keratin pair on a single chromosome is unique among keratin genes; it is consistent with the hypothesis that keratins 8 and 18 may be closer to an ancestral gene than the keratins of more highly differentiated epithelia. Casanova et al. (J Clin Invest. 103 (11):1587-95, 1999) generated mice expressing the human KRT8 gene, leading to a moderate increase in the content of keratin in simple epithelia. These mice displayed progressive exocrine pancreas alterations, including dysplasia and loss of acinar architecture, redifferentiation of acinar to ductal cells, inflammation, fibrosis, and substitution of exocrine by adipose tissue, as well as increased cell proliferation and apoptosis. The phenotype was very similar to that reported for transgenic mice expressing a dominant-negative mutant TGF-beta type II receptor. Casanova et al. (1999) showed that these Tgfbr2 mutant mice also had elevated KRT8/KRT18 levels. The results indicated that simple epithelial keratins play a relevant role in the regulation of exocrine pancreas homeostasis and supported the idea that disruption of mechanisms that normally regulate keratin expression in vivo could be related to inflammatory and neoplastic pancreatic disorders.

The NOV7 nucleic acid of the invention encoding a Cytokeratin-like protein includes the nucleic acid whose sequence is provided in Table 7A, or a fragment thereof. The invention also includes a mutant or variant nucleic acid any of whose bases may be changed from the corresponding base shown in Table 7A while still encoding a protein that maintains its Cytokeratin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 12% of the residues may be so changed.

The NOV7 protein of the invention includes the Cytokeratin-like protein whose sequence is provided in Table 7B. The invention also includes a mutant or variant protein any of whose residues may be changed from the corresponding residue shown in Table 7B while still encoding a protein that maintains its Cytokeratin-like activities and physiological functions, or a functional fragment thereof. In the mutant or variant protein, up to about 13% of the bases may be so changed.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from: Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection and other diseases, disorders and conditions of the like.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV8

NOV8 includes three novel Protocadherin-like proteins disclosed below. The disclosed proteins have been named NOV8a, NOV8b, and NOV8c.

NOV8a

A disclosed NOV8a nucleic acid of 2801 nucleotides (also referred to as CG57446-01) encoding a novel Protocadherin-like protein is shown in Table 8C. An open reading frame was identified beginning with an ATG initiation codon at nucleotides 6-8 and ending with a TAA codon at nucleotides 2724-2726. Putative untranslated regions, if any, upstream from the initiation codon and downstream from the termination codon are underlined in Table 8C, and the start and stop codons are in bold letters.

TABLE 8C
NOV8a nucleotide sequence (SEQ ID NO:25).

ACCGGATGCACCCGGAGATCTCCTACTCAATTCCTGAGGAAAGAGAGAAA
GGCTCTTTCGTGGGCAACATCTCCAAGGACTTGGGTCTGGCGCCCCGGGA
GCTGGCGGAGCGCGGAGTCCGCATACTCTCCAGAGGTAGGACGCAGCTTT
TCTCTCTGAACCCGCGCAGCGGCAGCTTGGTCACCGCGGGCAGGATAGAC
CGGGAGGAGCTCTGCGCTCAGAGCGCGCGGTGCGTGGTGAGTTTTAATAT
CCTTGTGGAAGACAGGGTGAAACTTTTTGGGATAGAAATAGAAGTAACTG
ATATCAATGACAATGCTCCAAAATTCCAAGCAGAAAATCTAGACGTAAAA
ATTAATGAAAATGTCGCTGCGGGAATGCGTTTTCCTCTCCCGGAAGCTAT
TGATCCGGATATAGGTGTAAACTCCCTGCAGGGTTACCAGCTCAACTCAA
ACGGTTACTTTTCCCTGGACGTGCAAAGTGGGGCCGATGGGATTAAGTAC
CCAGAGCTGGTGCTGGAACGCGCTCTAGATCGCGAGCAAGAGGCGGTTCA
CCACCTGGTCCTTACTGCCATGGATGGCGGCGACCCTCTCCGCTCAAGCG
TCGCCCAAATTCTGGTAACAGTTCTAGATGTGAATGACAACACTCCAATG
TTTACTCAGCCTGTCTACCGTGTAAGTGTTCCTGAAAACCTGCCAGCTCC
CGGAACTCGGGTGCTGATGGTTAATGCAACGGATCCAGATGAAGGAGTCA
ATGCGGAAGTAATGTATTCATTTCGGAAAGTCAGAGACGAAAGAGCACAG
CTATTACAGTTGTTTTATCTGAGTGCGGAGATAACGATAATGAGGGGTCT
GGAGGATGTGGACTATGGATACTATGACATAGATGACATAGACGATGAAG
GCCATGGTGTCCGTGCTAGAAGAGCGGTACGCAAGGTAGTGGTGGAAGTT
TTGGATGAAAATGACAACGCCCCAGAAATCACAGTCACCTCCGTCACCAC
CGCAGTCCCCGAAGCTGCTTCTGGAACTGCCATTATTTTCCTCAATGATA
GTGACCGAGAGGACGGGOGGAACAGTCCATTTATCAGTTCTGTCAATCCG
GGTCTTTCATTCAAAAAATTAGATAAAAAAGATGATTATTTCATTTTTAA
AACGACTCAAGACATAGACCGAAAAACTGTGTCCGAATACAACATCACCG
CAATAGCCCCAGAAACCCGAGCTCCTTCCCCTTCAACTCATATTACACTC
CTTGTGCTAGTGATCGACATCAATGAAAACCCTCCCCCTTATTCTCAATC
CTCCTACTACGTTTACGTAAACGAAAACAACGGCGCCGGCACTTCAATTA
TGACCGTTAATGACTCTGACCCCGATGACAATTCTAGTGTTATTTACTCC
TTGGCAGAGGCTACCCAAGGAGCTCCTCCCTCCTCCACCTATGCCTCTAT
CACATCAAACACTGGTGTGCTTGATGTGTTGTCCTTCTTCTACTATGAGT
ATTTTGATTTTCTGCAAATGCAGATGACGGCTAATGACAGTGGCAGCCCA
CCACTTAGCAGTAATGCGTCAATGAGATTGTTTGTGTTGGACAAGGATGA
CAATGCCCAAGACCTCCAGTACCCTGCCCCCCCCACTGGTGGTACTGCTG
TTGAGGTGCTGCCCCGCTCTGCAGCGCCTGGCTACCTGGTGACCAAGGTG
GTGGCAGTGGACGGAGACTCAGGCCAGAATGCTTGGCTCTCCTACCGCCT
ATTCGAGGCCAGTGAGCCGGGGCTCTTCTCGGTGGGGCTGCACACAGGTG
GAAAAGTGCGCACCGCTCGGGCCCTGCTAGATAGAGATGCGCTCAAACAG
AGCCTTGTGGTGGCTGTACAGGACCATGGCCAGCCCCCTCTCTCCGCCGC
CACCCTCACTGTGGCGGTTGCCGACGACTCCCTCCAAGACCTCGCGGATT
TCTTCGGCAGCCTCACGCCTTCAGAACACCAAGACGACTCCGGCCTCACA
CTCCTTCTTGTGGTAGTAGTGGCTGCAGTCTGCTTCGTCTTCCCGGTCTT
CGTCGTCGTGCTGCTAGTACTCAAGCTGAGGCGCTGGCACAAGTCCCGCC
TGCTTCACGCTGAAGGCAGCAGGTTGGCAGGGGTGGCTGCCTCCCACTTT
GGGGGCGTGGAGGGGGTTGGGGTTTTCCTGCCGAACTATTCCCACGAGGT
CTCCCTCACCGCGGACTCGCGGAAGAGCCCCCTGATCCCCACCCAACCCC
GCGCTGAAATTCCCCTCAGCAACCGGGAGAGTGGAGAGAGAAGCCGCCGT
CTGGTGATACTTAAGGATGTGCTTGAAACAGAGGGCGACCCTAGTGCACA
GCAAGCCCCGCCCAACACGGACTGGCGTTTCTCTCAGGCCCAGAGACCCG
GCACCAGCGGCTCCCAAAATGGCGATGACACCGGCACCTGGCCCAACAAC
CAGTTTGACACAGAGATGCTGCAAGCCATGATCTTGGCGTCCGCCAGTGA
AGCTGCTGATGGGAGCTCCACCCTGGGAGGGGGTGCCGGCACCATGGGAT
TGAGCGCCCGCTACGGACCCCAGTTCACCCTGCAGCACGTGCCCGACTAC
CGCCAGAATGTCTACATCCCAGGCAGCAATGCCACACTGACCAACGCAGC
TGGCAAGCGGGATGGCAAGGCCCCAGCAGGTGGCAATGGCAACAAGAAGA
AGTCGGGCAAGAAGGAGAAGAAGTAACATGGAGGCCAGGCCAAGAGCCAC
AGGGCGGCCTCTCCCCAACCAGCCCAGCTTCTCCTTACCTGCACCCAGGC
C

The disclosed NOV8a nucleic acid sequence, localized to chromsome 5, has 2214 of 2786 bases (79%) identical to a gb:GENBANK-ID:AF152324|acc:AF152324.1 mRNA from Homo sapiens (protocadherin gamma A4 (PCDH-gamma-A4) mRNA, complete cds) (E=0.0).

A NOV8a polypeptide (SEQ ID NO:26) encoded by SEQ ID NO:25 has 906 amino acid residues and is presented using the one-letter code in Table 8D. Signal P, Psort and/or Hydropathy results predict that NOV8a does not contain a signal peptide and is likely to be localized to the plasma membrane with a certainty of 0.7000 and to the nucleus with a certainty of 0.6000.

TABLE 8D
Encoded NOV8a protein sequence (SEQ ID NO:26).

MHPEISYSIPEEREKGSFVGNISKDLGLAPRELAERGVRIVSRGRTQLFS
LNPRSGSLVTAGRIDREELCAQSARCVVSFNILVEDRVKLFGIEIEVTDI
NDNAPKFQAENLDVKINENVAAGMRFPLPEAIDPDIGVNSLQGYQLNSNG
YFSLDVQSGADGIKYPELVLERALDREEEAVHHLVLTAMDGGDPLRSSVA
QILVTVLDVNDNTPMFTQPVYRVSVPENLPAPGTRVLMVNATDPDEGVNA
EVMYSTRKVRDERAQLLQLFYLSAETTIMRGLEDVDYGYYDIDDIDDEGH
GVRARRAVRKVVVEVLDENDNAPEITVTSVTTAVPEAASGTAIIFLNDSD
REDGGNSPFISSVNPGLSFKKLDKKDDYFIFKTTQDIDRKTVSEYNITAI
APETRAPSPSTHITLLVLVIDINENPPPYSQSSYYVYVNENNGAGTSIMT
VNDSDPDDNSSVIYSLAEATQGAPPSSTYASITSNTGVLDVLSFFYYEYF
DFLQMQMTANDSGSPPLSSNASMRLFVLDKDDNAQDLQYPAPPTGGTAVE
VLPRSAAPGYLVTKVVAVDGDSGQNAWLSYRLFEASEPGLFSVGLHTGGK
VRTARALLDRDALKQSLVVAVQDHGQPPLSAATLTVAVADDSLQDLADFF
GSLTPSEHQDDSGLTLLLVVVVAAVCFVFPVFVVVLLVLKLRRWHKSRLL
HAEGSRLAGVAASHFGGVEGVGVFLPNYSHEVSLTADSRKSPLIPTQPRA
EIPLSNRESGERSRRLVILKDVLETEGDPSGQQAPPNTDWRFSQAQRPGT
SGSQNGDDTGTWPNNQFDTEMLQAMILASASEAADGSSTLGGGAGTMGLS
ARYGPQFTLQHVPDYRQNVYIPGSNATLTNAAGKRDGKAPAGGNGNKKKS
GKKEKK

The NOV8a amino acid sequence has 635 of 903 amino acid residues (70%) identical to, and 721 of 903 amino acid residues (79%) similar to, the 931 amino acid residue ptnr:SPTREMBL-ACC:Q9Y5G9 protein from Homo sapiens (Human) (Protocadherin Gamma A4) (E=0.0).

The disclosed NOV8a is expressed in at least the following tissues: Uterus, colon, eye and retina. This information was derived by determining the tissue sources of the sequences that were included in the invention including but not limited to SeqCalling sources, Public EST sources, Literature sources, and/or RACE sources.

NOV8b

A disclosed NOV8b nucleic acid of 2836 nucleotides (also referred to as CG57444-01) encoding a novel Protocadherin-like protein is shown in Table 8E. An open reading frame was identified beginning with an ATG initiation codon at nucleotides 15-17 and ending with a TAA codon at nucleotides 2802-2804. Putative untranslated regions, if any, upstream from the initiation codon and downstream from the termination codon are underlined in Table 8E, and the start and stop codons are in bold letters.

TABLE 8E
NOV8b nucleotide sequence (SEQ ID NO:27).

AACCCGAGCGAACGATGGGAGGGAGCTGCGCGCAGAGGCGCCGGGCCGGC
CCGCGGCAGGTACTATTTGCTTTCCTGCTGCCTTTGTTCTACCCCACGCT
GTGTGAGCCGATCCGCTACTCGATTCCGGAGGAGCTGGCCAAGGGCTCGG
TGGTGGGGAACCTCGCTAAGGATCTAGGGCTTAGTGTCCTGGATGTGTCG
GCTCGCGAGCTGCGAGTGAGCGCGGAGAAGCTGCACTTCAGCGTAGACGC
GCAGAGCGGGGACTTACTTGTGAAGGACCGAATAGACCGTGAGCAAATAT
GCAAAGAGAGAAGAAGATGTGAGTTGCAATTGGAAGCTGTGGTGGAAAAT
CCTTTAAATATTTTTCATGTCATTGTGGTGATTGAGGATGTTAATGACCA
CGCCCCTCAATTCCGGAAAGATGAAATAAACTTAGAAATCAGTGAATCCG
TCAGCCTGGGGATGGGAACAATTCTTGAGTCTGCAGAAGATCCTGATATT
AGTATGAATTCGCTGAGCAAATACCAACTAAGTCCTAACGAGTATTTCTC
ATTGGTGGAGAAAGACAATCCTGATGGTGGCAAATATCCAGAATTAGTAT
TGCAGAAGACTCTGGACCGAGAAACGCAGAGCGCTCACCACTTGATATTG
ACCGCCTTGGACGGAGGGGACCCACCAAGAAGTGCCACCGCTCACATAGA
AATTTCTGTCAAGGATACCAATGATAACCCCCCGGTTTTCAGCAGAGACG
AATATAGAATTAGTCTTAGTGAAAATCTGCCCCCTGGGTCCCCTGTGTTG
CAAGTGACAGCCACTGACCAGGATGAGGGGGTCAATGCTGAGATAAACTA
CTACTTCCGAAGCACTGCCCAGAGCACAAAACATATGTTCTCATTGGATG
AGAAAACAGGTATGATTAAGAATAACCAGTCATTTGATTTTGAAGATGTA
GAAAGGTACACCATGGAAGCGGAAGCGAAGGACGGAGGTGGTCTCTCTAC
CCAGTGTAAAGTAATCATAGAAATCCTTGATGAAAACGACAACAGCCCAG
AAATAATCATCACTTCTCTCTCTGATCAGATTTTGGAGAATTCACCTCCA
GGAATGGTTGTTGCCCTCTTCAAAACACGGGATCTGGATTTCGGAGGAAA
TGGAGAAGTCAGGTGTAATATAGAAACAGACATTCCATTCAAGATTTATT
CTTCTTCCAATAACTACTACAAACTGGTGACAGATGGAGCCCTGGACCGA
GAGCAGACACCAGAATACAATGTCACCATCGTAGCCACTGACAGGGGCAA
GCCGCCTCTTTCTTCCAGTAGAAGCATCACCTTGTATGTCGCTGACATCA
ACGACAACGCCCCAGTTTTCGACCAGACGTCCTACGTGGTCCACGTGGCC
GAGAACAACCCGCCAGGAGCCTCCATTGCGCAAGTGAGCGCCTCTGACCC
GGATTTGGGGCTCAATGGCCACATCTCCTACTCTCTCATTGCCAGCGACC
TGGAGTCACGAACGCTGTCGTCCTACGTGTCCGTGAGCGCGCAGAGCGGG
GTGGTGTTCGCGCAGCGCGCCTTCGACCACGAGCAGCTGCGCGCCTTCGC
GCTCACGCTGCAGGCCCGCGACCAGGGCTCGCCCGCGCTCAGCGCGAACG
TGAGCCTGCGCGTGTTAGTGGACGACCGCAACGACAATGCGCCACGGGTG
CTGTACCCAGCTCTGGGTCCTGACGGCTCCGCGTTCTTCGATATGGTACC
TCGCTCTGCAGAGCCCGGCTACCTAGTGACTAAGGTGGTAGCGGTGGACG
CCGACTCGGGACACAACGCCTGGCTGTCCTACCACGTGCTGCAGGCCAGT
GAGCCCGGGCTCTTCAGCCTGGGGCTGCGAACAGGCGAGGTGCGCATGGT
GCGTGCTTTGGGTGACAAGGACTCGGTCCGCCAGCGCCTGCTAGTCGCTA
TAAGAGATGGAGGACAGCCACCCCTTTCAGCCACTGCCACGCTGCACCTG
GTGTTCGCAGATAGCTTGCAAGAGGTACTGCCGGATTTCAGCGACCATCC
CACACCCTCTGACTCCCAGGCTGAGATGCAGTTTTACCTGGTGGTGGCCT
TGGCCTTGATTTCTGTGCTCTTTCTCCTCGCGGTGATTCTAGCTATTGCT
CTACGCCTGCGACAGTCTTTCAGCCCTACTGCAGGAGACTGCTTTGAGTC
AGTTCTCTGCTCCAAGTCCGGACCTGTGGGTCCCCCCAACTACAGTGAGG
GAACGTTGCCCTATGCCTATAATTTTTGTGTGCCTGGGGATCAAATGAAT
CCAGAATTTAATTTTTTCACATCTGTTGATCATTGTCCAGCCACACAAGA
TAACCTCAACAAAGATAGCATGCTACTGGCTAGCATTTTAACTCCCAGCG
TTGAAGCAGATAAGAAGATTCTTAAACAGCAAGCCCCGCCCAACACGGAC
TGGCGTTTCTCTCAGGCCCAGAGACCCGGCACCAGCGGCTCCCAAAATGG
CGATGACACCGGCACCTGGCCCAACAACCAGTTTGACACAGAGATGCTGC
AAGCCATGATCTTGGCGTCCGCCAGTGAAGCTGCTGATGGGAGCTCCACC
CTGGGAGGGGGTGCCGGCACCATGGGATTGAGCGCCCGCTACGGACCCCA
GTTCACCCTGCAGCACGTGCCCGACTACCGCCAGAATGTCTACATCCCAG
GCAGCAATGCCACACTGACCAACGCAGCTGGCAAGCGGGATGGCAAGGCC
CCAGCAGGTGGCAATGGCAACAAGAAGAAGTCGGGCAAGAAGGAGAAGAA
GTAACATGGAGGCCAGGCCAAGAGCCACAGGGCGGC

The disclosed NOV8b nucleic acid sequence, localized to chromsome 5, has 2584 of 2822 bases (91%) identical to a gb:GENBANK-ID:AF152336|acc:AF152336.1 mRNA from Homo sapiens (protocadherin gamma B7 (PCDH-gamma-B7) mRNA, complete cds) (E=0.0).

A NOV8b polypeptide (SEQ ID NO:28) encoded by SEQ ID NO:27 has 929 amino acid residues and is presented using the one-letter code in Table 8F. Signal P, Psort and/or Hydropathy results predict that NOV8b does not contain a signal peptide and is likely to be localized to the plasma membrane with a certainty of 0.5140. The most likely cleavage site for a NOV8b polypeptide is between amino acids 30 and 31: TLC-EP

TABLE 8F
Encoded NOV8b protein sequence (SEQ ID NO:28).

MGGSCAQRRRAGPRQVLFPLLLPLEYPTLCEPIRYSIPEELAKGSVVGNL
AKDLGLSVLDVSARELRVSAEKLHFSVDAQSGDLLVKDRIDREQICKERR
RCELQLEAVVENPLNIFHVIVVIEDVNDHAPQFRKDEINLEISESVSLGM
GTILESAEDPDISMNSLSKYQLSPNEYFSLVEKDNPDGGKYPELVLQKTL
DRETQSAHHLILTALDGGDPPRSATAHIEISVKDTNDNPPVFSRDEYRIS
LSENLPPGSPVLQVTATDQDEGVNAEINYYFRSTAQSTKHMFSLDEKTGM
IKNNQSFDFEDVERYTMEVEAKDGGGLSTQCKVIIEILDENDNSPEIIIT
SLSDQILENSPPGMVVALFKTRDLDFGGNGEVRCNIETDIPFKIYSSSNN
YYKLVTDGALDREQTPEYNVTIVATDRGKPPLSSSRSITLYVADINDNAP
VFDQTSYVVHVAENNPPGASIAQVSASDPDLGLNGHISYSLIASDLESRT
LSSYVSVSAQSGVVFAQRAFDHEQLRAFALTLQARDQGSPALSANVSLRV
LVDDRNDNAPRVLYPALGPDCSAFFDMVPRSAEPGYLVTKVVAVDADSGH
NAWLSYHVLQASEPGLFSLGLRTGEVRMVRALGDKDSVRQRLLVAIRDGG
QPPLSATATLHLVFADSLQEVLPDFSDHPTPSDSQAEDQFYLVVALALIS
VLFLLAVILAIALRLRQSFSPTAGDCFESVLCSKSGPVGPPNYSEGTLPY
AYNPCVPGDQMNPEFNFFTSVDHCPATQDNLNKDSMLLASILTPSVEADK
KILRQQAPPNTDWRFSQAQRPGTSGSQNGDDTGTWPNNQFDTEMLQAMIL
ASASEAADGSSTLGGGAGTMGLSARYGPQFTLQHVPDYRQNVYIPGSNAT
LTNAAGKRDGKAPAGGNGNKKKSGKKEKK

The NOV8b amino acid sequence has 842 of 929 amino acid residues (90%) identical to, and 881 of 929 amino acid residues (94%) similar to, the 929 amino acid residue ptnr:SPTREMBL-ACC:Q9Y5F8 protein from Homo sapiens (Human) (Protocadherin Gamma B7) (E=0.0).

The disclosed NOV8b is expressed in at least the following tissues: Uterus, colon, eye and retina. This information was derived by determining the tissue sources of the sequences that were included in the invention including but not limited to SeqCalling sources, Public EST sources, Literature sources, and/or RACE sources.

NOV8c

A disclosed NOV8c nucleic acid of 2436 nucleotides (also referred to as CG57442-01) encoding a novel Protocadherin Beta-like protein is shown in Table 8G. An open reading frame was identified beginning with an ATG initiation codon at nucleotides 1-3 and ending with a TGA codon at nucleotides 2398-2400. Putative untranslated regions, if any, upstream from the initiation codon and downstream from the termination codon are underlined in Table 8G, and the start and stop codons are in bold letters.

TABLE 8G
NOV8c nucleotide sequence (SEQ ID NO:29).

ATGGCTGTCAGAGAGTTGTGCTTCCCAAGACAAAGGCAAGTCCTGTTTCT
TTTTCTTTTTTGGGGAGTGTCCTTGGCAGGTTCTGGGTTTGGACGTTATT
CGGTGACTGAGGAAACAGAGAAAGGATCCTTTGTGGTCAATCTGGCAAAG
GATCTGGGACTAGCAGAGGGGGAGCTGGCTGCAAGGGGAACCAGGGTGCT
TTCCGATGATAACAAACAATACCTGCTCCTGGATTCACATACCGGGAATT
TGCTCACAAATGAGAAACTGGACCGAGAGAAGCTGTGTGGCCCTAAAGAG
CCCTGTATGCTGTATTTCCAAATTTTAATGGATGATCCCTTTCAGATTTA
CCGGCCTGAGCTGAGAGTCAGGGATATAAATGATCACGCGCCAGTATTTC
AGGACAAAGAAACAGTCTTAAAAATATCAGAAAATACAGCTGAAGGGACA
GCATTTAGACTAGAAAGAGCACAGGATCCAGATGGAGGACTTAACGGTAT
CCAAAACTACACGATCAGCCCCAACTCTTTTTTCCATATTAACATTAGTG
GCGGTGATGAAGGCATGATATATCCAGAGCTAGTGTTGGACAAAGCACTG
GATCGGGAGGAGCAGGGAGAGCTCAGCTTAACCCTCACAGCGCTGGATGG
TGGGTCTCCATCCAGGTCTGGGACCTCTACTGTACGCATCGTTGTCTTGG
ACGTCAATGACAATGCCCCACAGTTTGCCCAGGCTCTGTATGAGACCCAG
GCTCCAGAAAACAGCCCCATTGGGTTCCTTATTGTTAAGGTATGGGCAGA
AGATGTAGACTCTGGAGTCAACGCGGAAGTATCCTATTCATTTTTTGATG
CCTCAGAAAATATTCGAACAACCTTTCAAATCAATCCTTTTTCTGGGGAA
ATCTTTCTCAGAGAATTGCTTGATTATGAGTTAGTAAATTCTTACAAAAT
AAATATACAGGCAATGGACGGTGGAGGCCTTTCTGCAAGATGTAGGGTTT
TAGTGGAAGTATTGGACACCAATGACAATCCCCCTGAACTGATCGTATCA
TCATTTTCCAACTCTGTTGCTGAGAATTCTCCTGAGACGCCGCTGGCTGT
TTTTAAGATTAATGACAGAGACTCTGGAGAAAATGGAAGGATGGTGTGCT
ACATTCAAGATGATCTGCCATTCCTACTAAAACCTTCTGTTGAGAATTTT
TACATCCTAATGACTGAAGGCGCGCTGGACAGAGAAGCAAGAGCTGAATA
TAATATCACCCTCACCGTCACAGATATGGGGACTCCAAGGCTGAAAACGG
AGCACAACATAACAGTGCAGATATCAGATGTCAATGATAACGCCCCCACT
TTCACCCAAACCTCCTACGCCCTGTTCGTCCGCGAGAACAACAGCCCCGC
CCTGCACATCGGCAGCGTCAGCGCCACAGACAGAGACTCAGGCACCAACG
CCCAGGTCACCTACTCGCTGCTGCCGCCCCAGGACCCGCACCTGCCCCTC
GCCTCCCTGGTCTCCATCAACGCAGACAACGGCCACCTGTTCGCCCTCAG
GTCGCTGGACTACGAGGCCCTGCAGGCTTTCGAGTTCCGCGTGGGCGCCA
CAGACCGCGGCTCCCCCGCGCTGAGCAGAGAGGCGCTGGTGCGCGTGCTG
GTGCTGGACGCCAACGACAACTCGCCCTTCGTGCTGTACCCGCTGCAGAA
CGGCTCCGCGCCCTGCACTGAGCTGGTGCCCCGGGCGGCCGAGCCGGGCT
ACCTGGTGACCAAGGTGGTGGCGGTGGACGGCGACTCGGGCCAGAACGCC
TGGCTGTCGTACCAGCTGCTCAAGGCCACGGAGCCCGGGCTGTTCGGTGT
GTGGGCGCACAATGGGGAGGTGCGCACCGCCAGGCTGCTGAGCGAGCGCG
ACGCAGCCAAGCACAGGCTCGTGGTGCTTGTCAAGGACAATGGCGAGCCT
CCTCGCTCGGCCACCGCCACGCTGCACTTGCTCCTGGTGGACGGCTTCTC
CCAGCCCTACCTGCCTCTCCCGGAGCCGGCCCCGGCCCAGGCCCAGGCCG
AGGCCGACTTGCTCACCGTCTACCTGGTGGTGGCGTTGGCCTCGGTGTCT
TCGCTCTTCCTCCTCTCGGTGCTCCTGTTCGTGGCGGTGCGGCTGTGCAG
GAGGAGCAGGGCGGCCTCGGTGGGTCGCTGCTCGGTGCCCGAGGGTCCTT
TTCCAGGGCATCTGGTGGACGTGAGCGGCACCGGGACCCTGTTCCAGAGC
TACCAGTACGAGGTGTGTCTGACTGGAGGTTCAGAGACCGGCGAGTTCAA
GTTCTTGAAGCCGATTACCCCCCACCTCCCGCCCCATAGGGGTGGGAAAG
AAATAGAGGAAAATTCTACTCTCCCCAATAGCTTTGGATTTAATTATTGA
AAGGAACCCACTTAATAAAGACATTTACTTCTTTAA

The disclosed NOV8c nucleic acid sequence, localized to chromsome 5, has 2319 of 2400 bases (96%) identical to a gb:GENBANK-ID:AF152489|acc:AF152489.1 mRNA from Homo sapiens (Homo sapiens protocadherin beta 10 (PCDH-beta10) mRNA, complete cds) (E=0.0).

A NOV8c polypeptide (SEQ ID NO:30) encoded by SEQ ID NO:29 has 799 amino acid residues and is presented using the one-letter code in Table 8H. Signal P, Psort and/or Hydropathy results predict that NOV8c does not contain a signal peptide and is likely to be localized to the plasma membrane with a certainty of 0.4600. The most likely cleavage site for a NOV8c polypeptide is between amino acids 26 and 27: SLA-GS.

TABLE 8H

Encoded NOV8c protein sequence (SEQ ID NO:30).

MAVRELCEPRQRQVLELFLFWGVSLAGSGEGRYSVTEETEKGSFVVNLAK
DLGLAEGELAARGTRVVSDDNKQYLLLDSHTGNLLTNEKLDREKLCGPKE
PCMLYFQILMDDPFQIYRAELRVRDINDHAPVFQDKETVLKISENTAEGT
AFRLERAQDPDGGLNGIQNYTISPNSFFHINISGGDEGMIYPELVLDKAL
DREEQGELSLTLTALDGGSPSRSGTSTVRIVVLDVNDNAPQFAQALYETQ
APENSPIGFLIVKVWAEDVDSGVNAEVSYSFFDASENIRTTFQINPFSGE
IFLRELLDYELVNSYKINIQAMDGGGLSARCRVLVEVLDTNDNPPELIVS
SESNSVAENSPSTPLAVFKINDRDSGENGRNVCYIQDDLPFLLKPSVENE
YILMTEGALDREARAEYNITLTVTDMGTPRLKTEHNITVQISDVNDNAPT
FTQTSYALFVRENNSPALHIGSVSATDRDSGTNAQVTYSLLPPQDPHLPL
ASLVSINADNGHLFALRSLDYEALQAFEFRVGATDRGSPALSREALVRVL
VLDANDNSPFVLYPLQNGSAPCTELVPRAAEPGYLVTKVVAVDGDSGQNA
WLSYQLLKATEPGLFGVWAHNGEVRTARLLSERDAAKHRLVVLVKDNGEP
PRSATATLHLLLVDGFSQPYLPLPEAAPAQAQAEADLLTVYLVVALASVS
SLFLLSVLLFVAVRLCRRSRAASVGRCSVPEGPFPGHLVDVSGTGTLFQS
YQYEVCLTGGSETGEEKFLKPITPHLPPHRGGKEIEENSTLPNSFGFNY

The NOV8c amino acid sequence has 765 of 798 amino acid residues (95%) identical to, and 776 of 798 amino acid residues (97%) similar to, the 800 amino acid residue ptnr:SPTREMBL-ACC:Q9UN67 protein from Homo sapiens (Human) (Protocadherin Beta 10) (E=0.0).

The disclosed NOV8c is expressed in at least the following tissues: Uterus, colon, eye and retina. This information was derived by determining the tissue sources of the sequences that were included in the invention including but not limited to SeqCalling sources, Public EST sources, Literature sources, and/or RACE sources.

Possible SNPs found for NOV8b are listed in Table 8I.

TABLE 8I
SNPs

| | | | Amino | |
|---|---|---|---|---|
| | Nucleotide | Base | Acid | Base |
| Variant | Position | Change | Position | Change |
| 13377099 | 218 | G > T | Silent | N/A |

NOV8a-NOV8c are very closely homologous genes. Homologies to any of the above NOV8 proteins will be shared by the other NOV8 proteins insofar as they are homologous to each other as shown above. Any reference to NOV8 is assumed to refer to the NOV8 proteins in general, unless otherwise noted.

Cadherins, a family of calcium-dependent cell-cell adhesion molecules, mediate neural cell-cell interactions. Sperry (1963) proposed that neurons recognize their synaptic partners through lock-and-key interactions mediated by millions of specifier molecules. Cadherins were likely candidates for components of the lock-and-key mechanism based on their neural location, their adhesive diversity, and the structural biology of their adhesive interactions maintaining a synapse gap size of approximately 200 angstroms (Shapiro and Colman, Neuron. 23 (3):427-30, 1999). Neural cadherins (CDH2, 114020), as well as epithelial (CDH1, 192090), placental (CDH3, 114021), and retinal (CDH4, 603006) cadherins, have homophilic binding specificities in that they preferentially adhere to cells expressing the same cadherin type. Cadherins of the ‘classic’ type have a highly conserved extracellular sequence motif of approximately 110 amino acids that is repeated 5 times as well as a highly conserved cytoplasmic domain of approximately 200 amino acids that associates with intracellular actin microfilaments via catenins (see CTNNA1). ‘Nonclassic’ cadherins differ in that they may have 6 or 7 repeated extracellular domains or have cytoplasmic domains that connect to intermediate filaments instead of actin.

Protocadherins constitute a subfamily of the nonclassic cadherins. Kohmura et al. (Neuron. 20 (6):1137-51, 1998) described cadherin-related neuronal receptors (CNR) in the mouse. By EST database searching for cadherin-like sequences, Wu and Maniatis (Cell 97 (6):779-90, 1999) identified 52 novel genes organized into 3 closely linked tandem clusters on human chromosome 5q31. A distinct large exon of approximately 2,400 nucleotides encodes the 6 N-terminal extracellular domains and the transmembrane domain of each cadherin. In contrast, the C terminus of each of these proteins is identical within each cluster and is encoded by 3 small exons located downstream from the array of N-terminal exons. Wu and Maniatis (1999) designated the clusters alpha, beta and gamma. Each large exon is independently spliced to the first exon encoding the intracellular domain. The authors also denoted the extracellular portion as the variable region and the cytoplasmic portion as the constant region. The alpha cluster contains at least 15 proteins encoded by large, uninterrupted exons whose sequences most closely resemble those of the mouse CNR proteins. Wu and Maniatis (1999) proposed 4 models to explain protocadherin gene regulation and noted that several neurologic disorders map to chromosome 5q31.

The NOV8 nucleic acid of the invention encoding a Protocadherin-like protein includes the nucleic acid whose sequence is provided in Tables 8C, 8E and 8G, or a fragment thereof. The invention also includes a mutant or variant nucleic acid any of whose bases may be changed from the corresponding base shown in Tables 8C, 8E and 8G while still encoding a protein that maintains its Protocadherin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 21% of the NOV8a residues, about 9% of the NOV8b residues and about 4% of the NOV8c residues may be so changed.

The NOV8 protein of the invention includes the Protocadherin-like protein whose sequence is provided in Tables 8D, 8F and 8H. The invention also includes a mutant or variant protein any of whose residues may be changed from the corresponding residue shown in Tables 8D, 8F and 8H while still encoding a protein that maintains its Protocadherin-like activities and physiological functions, or a functional fragment thereof. In the mutant or variant protein, up to about 30% of the NOV8a bases, about 10% of the NOV8b bases and about 5% of the NOV8c bases may be so changed.

The NOV8 nucleic acids and proteins of the invention are useful in potential diagnostic and therapeutic applications implicated in various diseases and disorders described below and/or other pathologies. For example, the compositions of the present invention will have efficacy for treatment of patients suffering from: Hirschsprung's disease, Crohn's Disease, Appendicitis, Von Hippel-Lindau (VHL) syndrome, Diabetes, Tuberous sclerosis, Endometriosis, Fertility and other diseases, disorders and conditions of the like.

NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV8 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOV9

A disclosed NOV9 nucleic acid of 1327 nucleotides (also referred to as CG57484-01) encoding a novel Keratin 18-like protein is shown in Table 9A. An open reading frame was identified beginning with an ATG initiation codon at nucleotides 67-69 and ending with a TAA codon at nucleotides 1273-1275. Putative untranslated regions upstream from the intitation codon and downstream from the termination codon are underlined in Table 9A, and the start and stop codons are in bold letters.

TABLE 9A
NOV9 Nucleotide Sequence (SEQ ID NO:31)

GCTTCACCACTCCCTCCACCTTCTCCACCAACTACCAGTCCCTGGGCTCT
GTCCAGCCGCCCAGCTATGGCACCTGGCCGGTCAGCAGCGCAGCCAGCAT
CTATGCAGGCACTGGGGGGCTTGGGATCCCAGATCTCCATGTCCTGTTCT
ACCAGTTTCTGGGGCGGCTTGGGGTCTGGGGGCCTGGCCACAGAGATGGC
TGGGGGTCTGGCAGAAATGGGGGGCATCCAGAATGAGAAGGAGACCATGC
AAAGCCTGAACGACCACCTGGACTACCTGGACAGAGTGAGGAACCTGGAG
ACCGAGAACTGGAGGCTGGAGAGCAAAATCCAGGAGTATCTGGAGAAGAG
ACCCCATGTCAGAGACTGGGGCCATTACTTCAAGACCATCAAGGAACTGA
GGGCTCAGATCTTCGCAAATACTGTGGACAATGTCCACATCATTCTGCAG
ATCGACAATGCCCGTCTTGCTGCTGATGACTTCAGAGTCAAGTATGAGAC
AAGAGCTGGCCATGCGCCAGTCTGTGGAGAGAACATCCATGGGCTCTGCA
AGGTCATTGATGACACCAATGTCACTCTGCTGCAGCTGGAGACAGAGATG
GGCGCTCTCAAGGAGGAGCTGCTCCTCATGAAGAAGAACCATGAAGAGGA
AGTAAAAGGCTTGCAAGTCCAGATTGCCAACTCTGGGTTGGCCGTGGAGG
TAGATGCCCCCAAATCTCAAGTCCTCGCCAAGGTCATGGCAGACATCAGG
GCCCAAGATGAGCTGTCTCAGAAGAACTCAGAGAAGCTAGGCAAGTACTG
GTCTCAGCAGACTGAGGAGAGCACCACAGTGGTCACCACACACTCTGCCA
AGGTCAGAGCTGCTGAGATGACAACGGAGCTGAGACGTACAGTCCAGTGC
TTGGAGATTGACCTGGACTCAATGAGAAATCTGAAGACCAGCTTGAACAG
CCTGAGGGAGGTGGAGGCCCGCTACGCCCTGCAGATGGAGCAGCTCAACA
GAATCCTGCTGTACTTGGAGTCAAAGCTGGCACAGAACTGGGCAGAGGGC
CAGCGCAAGGTCCAGGAGTACAAGGACTTGCTGAACATCAGGGTCAAGCT
GGAGGCTGAGATCGCCACCTACCGCCGCCTGCTGGAAGACAGCGAGGGCC
TCAATCTTGGTGATGCCCTGGACAGCAGCAACTCCATGCAAACCATCCAA
AAGACCACCACCCGCCAGATAGTGGATAGCAAAGTGGTGTCTGAGATCAG
TGACACCAAAGTTCTGAGACATTAAGCCAGCAGAAGCAGGGTACCCTGTG
GGGAGTAAGAGGCCAATAAAAAGTTCA

The NOV9 nucleic acid was identified on chromosome 12 and has 367 of 415 bases (88%) identical to a gb:GENBANK-ID:AF179904|acc:AF179904.1 mRNA from Homo sapiens (Homo sapiens keratin 18 (KRT18) gene, complete cds) (E=5.1e⁻¹⁸⁰).

A disclosed NOV9 polypeptide (SEQ ID NO:32) encoded by SEQ ID NO:31 is 402 amino acid residues and is presented using the one-letter code in Table 9B. Signal P, Psort and/or Hydropathy results predict that NOV9 contains a signal peptide and is likely to be localized to the cytoplasm with a certainty of 0.4500.

TABLE 9B
Encoded NOV9 protein sequence (SEQ ID NO:32)

MAPGRSAAQPASMQALGGLGSQISMSCSTSFWGGLGSGGLATEMAGGLAE
MGGIQNEKETMQSLNDHLDYLDRVRNLETENWRLESKIQEYLEKRPHVRD
WGHYFKTIKELRAQIFANTVDNVHIILQIDNARLAADDFRVKYETRAGHA
PVCGENIHGLCKVIDDTNVTLLQLETEMGALKEELLLMKKNHEEEVKGLQ
VQIANSGLAVEVDAPKSQVLAKVMADIRAQDELSQKNSEKIGKYWSQQTE
ESTTVVTTHSAKVRAAEMTTELRRTVQCLEIDLDSMRNLKTSLNSLREVE
ARYALQMEQLNRILLYLESKLAQNWAEGQRKVQEYKDLLNIRVKLEAEIA
TYRRLLEDSEGLNLGDALDSSNSMQTIQKTTTRQIVDSKVVSEISDTKVL
RH

The NOV9 amino acid sequence 315 of 404 amino acid residues (77%) identical to, and 344 of 404 amino acid residues (85%) similar to, the 429 amino acid residue ptnr:SWISSNEW-ACC:P05783 protein from Homo sapiens (Human) (Keratin, Type I Cytoskeletal 18 (Cytokeratin 18) (K18) (CK 18)) (E=2.9e⁻¹⁵¹).

NOV9 is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. This information was derived by determining the tissue sources of the sequences that were included in the invention including but not limited to SeqCalling sources, Public EST sources, genomic clone sources; literature sources, and/or RACE sources.

NOV9 has homology to the amino acid sequences shown in the BLASTP data listed in Table 9C.

TABLE 9C
BLAST results for NOV9

| Gene Index/ | | Length | Identity | Positives | |
|---|---|---|---|---|---|
| Identifier | Protein/Organism | (aa) | (%) | (%) | Expect |
| gi\|4557888\|ref\|NP_000215.1\| | keratin 18 [Homo | 430 | 303/404 | 332/404 | e−142 |
| (NM_000224) | sapiens] | | (75%) | (82%) | |
| gi\|106851\|pir\|\|S06889 | keratin 18, | 424 | 303/404 | 332/404 | e−142 |
| | cytoskeletal | | (75%) | (82%) | |
| | [Homo sapiens] | | | | |
| gi\|12653819\|gb\|AAH00698.1\| | keratin 18 [Homo | 430 | 302/404 | 332/404 | e−141 |
| AAH00698 | sapiens] | | (74%) | (81%) | |
| (BC000698) | | | | | |
| gi\|14602495\|gb\|AAH09754.1\| | Similar to | 375 | 286/372 | 310/372 | e−132 |
| AAH09754 | keratin 18 [Homo | | (76%) | (82%) | |
| (BC009754) | sapiens] | | | | |
| gi\|532610\|gb\|AAA37552.1\| | cytokeratin [Mus | 423 | 268/405 | 313/405 | e−121 |
| (M22832) | musculus] | | (66%) | (77%) | |

Table 9E lists the domain description from DOMAIN analysis results against NOV9. This indicates that the NOV9 sequence has properties similar to those of other proteins known to contain these domains.

TABLE 9E

Domain Analysis of NOV9
gnl|pfam|pfam00038, filament, Intermediate filament protein.
(SEQ ID NO: 71)
CD-Length = 312 residues, 99.4% aligned
Score = 204 bits (520), Expect = 6e−54

| | |
|---|---|
| Query: 56 | NEKETMQSLNDHL-DYLDRVRNLETENWRLESKIQEYLEKRPHV--RDWGHYFKTIKELR 112 |
| | \|\|\|\| \|\|+\|\|\| \| \|+\|+\|\| \|\| +\| \|\| \|\|+\| +\|+ \| + \| \|+\|\|\| |
| Sbjct: 1 | NEKEQMQNLNDRLASYIDKVRFLEQQNKELEVKIEELRQKQAPSVSRLYSLYETEIEELR 60 |
| Query: 113 | AQIFANTVDNVHIILQIDNARLAADDFRVKYETRAGHAPVCGENIHGLCKVIDDTNVTLL 172 |
| | \|\| \| + + \|+\|\|\| \| \|\|+\|\|\| \|\|\| ++ \|\| \| +\|+ + + |
| Sbjct: 61 | RQIDQLTNERARLQLEIDNLREAAEDFRKKYEDEINLRQEAENDLVGLRKDLDEATLARV 120 |
| Query: 173 | QLETEMGALKEELLLDKKHEEEVKGLQVQIANSGLAVEVDAPKSQVLAKVIAADIRAQ-D 231 |
| | \|\| ++ +\|+\|\|\| +\|\|\|\|\|\|\|\|\| \|\| \|\| ++ \|\|+\|\| + \| \| + +\|\|\|\| + |
| Sbjct: 121 | DLENKVESLQEELEFLKKNHEEEVKELQAQIQDTVN-VEMDAARKLDLTKALREIRAQYE 179 |
| Query: 232 | ELSQKNSEKLGKYWSQQTEESTTVVTTHSAKVRAA-EMTTELRRTVQCLEIDLDSMRNLK 290 |
| | \|+++\|\| ++ +++ + \|\| \| + +\|+\| \| \|\|\|\|\| +\| \|\|\|+\| \|++ |
| Sbjct: 180 | EIAXKNRQEAEEWYKSKLEELQTAAARNGEALRSAKEEITELRRQIQSLEIELQSLKAQN 239 |
| Query: 291 | TSL-NSLREVEARYALQDEQLNRILLYLESKLAQNWAEGQRKVQEYKDLLNIRVKLEAEI 349 |
| | \|\| \| \|+\| \|\| \|++ \| ++ \|\| +\| \| \| \|+++\|\|++\|\|++++ \|+ \|\| |
| Sbjct: 240 | ASLERQLAELEERYELELRQYQALISQLEEELQQLREEMARQLREYQELLDVKLALDIEI 299 |
| Query: 350 | ATYRRLLEDSE 360 |
| | \|\|\|\|+\|\|\| \| |
| Sbjct: 300 | ATYRKLLEGEE 310 |

Keratin 8 (K8) and keratin 18 (K18) are the most common and characteristic members of the large intermediate filament gene family expressed in ‘simple’ or single layer epithelial tissues of the body. Their persistent expression in tumor cells derived from these epithelia has led to the wide spread use of keratin monoclonal antibodies as aids in the detection and identification of carcinomas. Oncogenes which activate ras signal transduction pathways stimulate expression of the K18 gene through transcription factors including members of the AP-1 (jun and fos) and ETS families. The persistent expression of K8 and K18 may reflect the integrated transcriptional activation of such transcription factors and, in the cases of ectopic expression, an escape from the suppressive epigenetic mechanisms of DNA methylation and chromatin condensation. Comparison of the mechanisms of transcriptional control of K18 expression with expression patterns documented in both normal and pathological conditions leads to the proposal that persistent K8 and K18 expression is a reflection of the action of multiple different oncogenes converging on the nucleus through a limited number of transcription factors to then influence the expression of a large number of genes including these keratins. Furthermore, correlation of various tumor cell characteristics including invasive behavior and drug sensitivity with K8 and K18 expression has stimulated consideration of the possible functions of these proteins in both normal development and in tumorigenesis. Recent developments in the analysis of the functions of these intermediate filament proteins provide new insights into diverse functions influenced by K8 and K18 (Oshima et al., Cancer Metastasis Rev 15:445-71, 1996).

Keratin 8 (K8) and keratin 18 (K18) form intermediate filaments characteristic of liver and other single cell layered, internal epithelia and their derivative cancers. K8-deficient (K8(−)) mice, which escape embryonic lethality, develop inflammatory colorectal hyperplasia, mild liver abnormalities, and tolerate hepatectomy poorly. Normal and malignant epithelial cells deficient in K8 and K18 have been shown to be approximately 100 times more sensitive to TNF-induced death. K8 and K18 both bind the cytoplasmic domain of TNFR2 and moderate TNF-induced, Jun NH(2)-terminal kinase (JNK) intracellular signaling and NFkappaB activation. Furthermore, K8(−) and K18(−) mice are much more sensitive to TNF dependent, apoptotic liver damage induced by the injection of concanavalin A. This moderation of the effects of TNF may be the fundamental function of K8 and K18 common to liver regeneration, inflammatory bowel disease, hepatotoxin sensitivity, and the diagnostic, persistent expression of these keratins in many carcinomas (Caulin et al., J Cell Biol 149:17-22, 2000).

The NOV9 nucleic acid of the invention encoding a Keratin 18-like protein includes the nucleic acid whose sequence is provided in Table 9A, or a fragment thereof. The invention also includes a mutant or variant nucleic acid any of whose bases may be changed from the corresponding base shown in Table 9A while still encoding a protein that maintains its Keratin 18-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 12% of the NOV9 residues may be so changed.

The NOV9 protein of the invention includes the Keratin 18-like protein whose sequence is provided in Table 9B. The invention also includes a mutant or variant protein any of whose residues may be changed from the corresponding residue shown in Table 9B while still encoding a protein that maintains its Keratin 18-like activities and physiological functions, or a functional fragment thereof. In the mutant or variant protein, up to about 23% of the NOV9 bases may be so changed.

The NOV9 nucleic acids and proteins of the invention are useful in potential diagnostic and therapeutic applications implicated in various diseases and disorders described below and/or other pathologies. For example, the compositions of the present invention will have efficacy for treatment of patients suffering from: inflammatory colorectal hyperplasia, mild liver abnormalities, skin abnormalities, inflammatory bowel disease, hepatotoxin sensitivity, chronic hepatitis, breast cancer, pancreatic cancer, mammary gland tumours, benign prostatic hyperplasia, prostate cancer, cryptogenic cirrhosis and other diseases, disorders and conditions of the like.

NOV9 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV9 protein have multiple hydrophilic regions, each of which can be used as an immunogen. This novel protein also has value in development of powerful assay system for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NO:1, 3, 5, 7, 9, 11, 13, 15, 17, 19, 21, 23, 25, 27, 29, or 31, or a complement of this aforementioned nucleotide sequence, can be isolated using standard molecular biology techniques and the sequence information provided herein. Using all or a portion of the nucleic acid sequence of SEQ ID NOs:1, 3, 5, 7, 9, 11, 13, 15, 17, 19, 21, 23, 25, 27, 29, or 31 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:1, 3, 5, 7, 9, 11, 13, 15, 17, 19, 21, 23, 25, 27, 29, or 31, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:1, 3, 5, 7, 9, 11, 13, 15, 17, 19, 21, 23, 25, 27, 29, or 31, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown in SEQ ID NO:1, 3, 5, 7, 9, 11, 13, 15, 17, 19, 21, 23, 25, 27, 29, or 31 is one that is sufficiently complementary to the nucleotide sequence shown SEQ ID NO:1, 3, 5, 7, 9, 11, 13, 15, 17, 19, 21, 23, 25, 27, 29, or 31 that it can hydrogen bond with little or no mismatches to the nucleotide sequence shown SEQ ID NO:1, 3, 5, 7, 9, 11, 13, 15, 17, 19, 21, 23, 25, 27, 29, or 31, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOs:1, 3, 5, 7, 9, 11, 13, 15, 17, 19, 21, 23, 25, 27, 29, and 31, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NO:1, 3, 5, 7, 9, 11, 13, 15, 17, 19, 21, 23, 25, 27, 29, or 31; or an anti-sense strand nucleotide sequence of SEQ ID NO:1, 3, 5, 7, 9, 11, 13, 15, 17, 19, 21, 23, 25, 27, 29, or 31; or of a naturally occurring mutant of SEQ ID NO:1, 3, 5, 7, 9, 11, 13, 15, 17, 19, 21, 23, 25, 27, 29, or 31.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NO:1, 3, 5, 7, 9, 11, 13, 15, 17, 19, 21, 23, 25, 27, 29, or 31, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOs:1, 3, 5, 7, 9, 11, 13, 15, 17, 19, 21, 23, 25, 27, 29, and 31 due to degeneracy of the genetic code and thus encode the same NOVX proteins as that encoded by the nucleotide sequences shown in SEQ ID NOs:1, 3, 5, 7, 9, 11, 13, 15, 17, 19, 21, 23, 25, 27, 29, and 31. In another embodiment, an isolated nucleic acid molecule of the invention has a nucleotide sequence encoding a protein having an amino acid sequence shown in SEQ ID NO:2, 4, 6, 8, 10, 12, 14, 16, 18, 20, 22, 24, 26, 28, 30, or 32.

In addition to the human NOVX nucleotide sequences shown in SEQ ID NOs:1, 3, 5, 7, 9, 11, 13, 15, 17, 19, 21, 23, 25, 27, 29, and 31, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOs:1, 3, 5, 7, 9, 11, 13, 15, 17, 19, 21, 23, 25, 27, 29, and 31 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, 3, 5, 7, 9, 11, 13, 15, 17, 19, 21, 23, 25, 27, 29, or 31. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOs:1, 3, 5, 7, 9, 11, 13, 15, 17, 19, 21, 23, 25, 27, 29, and 31, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:1, 3, 5, 7, 9, 11, 13, 15, 17, 19, 21, 23, 25, 27, 29, or 31, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOs:1, 3, 5, 7, 9, 11, 13, 15, 17, 19, 21, 23, 25, 27, 29, and 31, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOs:1, 3, 5, 7, 9, 11, 13, 15, 17, 19, 21, 23, 25, 27, 29, and 31, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOs:2, 4, 6, 8, 10, 12, 14, 16, 18, 20, 22, 24, 26, 28, 30, or 32. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOs:1, 3, 5, 7, 9, 11, 13, 15, 17, 19, 21, 23, 25, 27, 29, and 31 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOs:2, 4, 6, 8, 10, 12, 14, 16, 18, 20, 22, 24, 26, 28, 30, and 32. Preferably, the protein encoded by the nucleic acid molecule is at least about 60% homologous to SEQ ID NOs:2, 4, 6, 8, 10, 12, 14, 16, 18, 20, 22, 24, 26, 28, 30, and 32; more preferably at least about 70% homologous SEQ ID NOs:2, 4, 6, 8, 10, 12, 14, 16, 18, 20, 22, 24, 26, 28, 30, and 32; still more preferably at least about 80% homologous to SEQ ID NOs:2, 4, 6, 8, 10, 12, 14, 16, 18, 20, 22, 24, 26, 28, 30, and 32; even more preferably at least about 90% homologous to SEQ ID NOs:2, 4, 6, 8, 10, 12, 14, 16, 18, 20, 22, 24, 26, 28, 30, and 32; and most preferably at least about 95% homologous to SEQ ID NOs:2, 4, 6, 8, 10, 12, 14, 16, 18, 20, 22, 24, 26, 28, 30, and 32.

An isolated nucleic acid molecule encoding an NOVX protein homologous to the protein of SEQ ID NOs:2, 4, 6, 8, 10, 12, 14, 16, 18, 20, 22, 24, 26, 28, 30, and 32 can be created by introducing one or more nucleotide substitutions, additions or deletions into the nucleotide sequence of SEQ ID NOs:1, 3, 5, 7, 9, 11, 13, 15, 17, 19, 21, 23, 25, 27, 29, and 31, such that one or more amino acid substitutions, additions or deletions are introduced into the encoded protein.

Mutations can be introduced into SEQ ID NOs:1, 3, 5, 7, 9, 11, 13, 15, 17, 19, 21, 23, 25, 27, 29, and 31 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOs: 1, 3, 5, 7, 9, 11, 13, 15, 17, 19, 21, 23, 25, 27, 29, and 31, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOs:1, 3, 5, 7, 9, 11, 13, 15, 17, 19, 21, 23, 25, 27, 29, and 31, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOs:2, 4, 6, 8, 10, 12, 14, 16, 18, 20, 22, 24, 26, 28, 30, and 32, or antisense nucleic acids complementary to an NOVX nucleic acid sequence of SEQ ID NOs:1, 3, 5, 7, 9, 11, 13, 15, 17, 19, 21, 23, 25, 27, 29, and 31, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′-and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Examples of modified nucleotides that can be used to generate the antisense nucleic acid are well known in the art and are not described in detail here.

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual 13-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15:6131-6148) or a chimeric RNA-DNA analogue (See, e.g., Inoue, et al., 1987. FEBS Lett. 215:327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein. For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′PNA segment and a 3′DNA segment. See, e.g., Finn, et al., 1996. supra. Alternatively, chimeric molecules can be synthesized with a 5′DNA segment and a 3′PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOs:2, 4, 6, 8, 10, 12, 14, 16, 18, 20, 22, 24, 26, 28, 30, and 32. The invention also includes a mutant or variant protein any of whose residues may be changed from the corresponding residues shown in SEQ ID NOs:2, 4, 6, 8, 10, 12, 14, 16, 18, 20, 22, 24, 26, 28, 30, and 32 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOs:2, 4, 6, 8, 10, 12, 14, 16, 18, 20, 22, 24, 26, 28, 30, and 32. In other embodiments, the NOVX protein is substantially homologous to SEQ ID NOs:2, 4, 6, 8, 10, 12, 14, 16, 18, 20, 22, 24, 26, 28, 30, and 32, and retains the functional activity of the protein of SEQ ID NOs:2, 4, 6, 8, 10, 12, 14, 16, 18, 20, 22, 24, 26, 28, 30, and 32, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOs:2, 4, 6, 8, 10, 12, 14, 16, 18, 20, 22, 24, 26, 28, 30, and 32, and retains the functional activity of the NOVX proteins of SEQ ID NOs:2, 4, 6, 8, 10, 12, 14, 16, 18, 20, 22, 24, 26, 28, 30, and 32.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOs:1, 3, 5, 7, 9, 11, 13, 15, 17, 19, 21, 23, 25, 27, 29, and 31.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOs:2, 4, 6, 8, 10, 12, 14, 16, 18, 20, 22, 24, 26, 28, 30, and 32, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NOs:2, 4, 6, 8, 10, 12, 14, 16, 18, 20, 22, 24, 26, 28, 30, and 32, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human SECX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13:65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. See e.g., Milstein and Cuello, Nature, 305:537-539 (1983); WO 93/08829, published 13 May 1993; and Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148 (5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81 (19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1 (1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, Triton® X-100, Thesit®, Isotridecypoly(ethylene glycol ether)ₙ, or 3-(3-cholamidopropyl) dimethylamminiol-1-propane sulfonate (CHAPS).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOs:1, 3, 5, 7, 9, 11, 13, 15, 17, 19, 21, 23, 25, 27, 29, and 31, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOs:1, 3, 5, 7, 9, 11, 13, 15, 17, 19, 21, 23, 25, 27, 29, and 31 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:1, 3, 5, 7, 9, 11, 13, 15, 17, 19, 21, 23, 25, 27, 29, or 31, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence.

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. Cotton, et al, 1988. Proc. Natl. Acad. Sci. USA 85: 4397; Saleeba, et al., 1992; and Methods Enzymol. 217: 286-295. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. See, e.g., Hsu, et al., 1994. Carcinogenesis 15: 1657-1662, and U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. See, e.g., Saiki, et al., 1986. Nature 324: 163; Saiki, et al., 1989. Proc. Natl. Acad. Sci. USA 86: 6230.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (e.g., metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers). In conjunction with such treatment, the pharmacogenomics (i.e., the study of the relationship between an individual's genotype and that individual's response to a foreign compound or drug, see e.g., Eichelbaum, 1996. Clin. Exp. Pharmacol. Physiol., 23: 983-985; Linder, 1997. Clin. Chem., 43: 254-266) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

The activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

### Example 3

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5 ′ and 3 ′ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers: Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

−M=Male

−F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. NOV1: Androgen-Regulated Short-Chain Dehydrogenase/Reductase

Expression of gene CG57413-01 was assessed using the primer-probe set Ag3230, described in Table AA. Results of the RTQ-PCR runs are shown in Tables AB, AC, AD and AE.

TABLE AA

Probe Name Ag3230

| | | | |
|---|---|---|---|
| | | | Start |
| Pri- | | | Posi- |
| mers | Sequences | Length | tion |

| | | | |
|---|---|---|---|
| For- | 5′-gatggtacgtgatgctcttgtt-3′ | 22 | 10 |
| ward | (SEQ ID NO:33) | | |
| Probe | TET-5′- | | |
| | cccttgccgataggaagttctttgct-3′- | 26 | 33 |
| | TAMRA (SEQ ID NO:34) | | |
| Re- | 5′-aggaagctgcacatttgttcta-3′ | 22 | 70 |
| verse | (SEQ ID NO:35) | | |

TABLE AB
CNS_neurodegeneration_v1.0

| | Rel. Exp.(%) Ag3230, Run | | Rel. Exp.(%) Ag3230, Run |
|---|---|---|---|
| Tissue Name | 206533576 | Tissue Name | 206533576 |
| AD 1 Hippo | 0.0 | Control (Path) 3 | 0.0 |
| | | Temporal Ctx | |
| AD 2 Hippo | 0.0 | Control (Path) 4 | 54.7 |
| | | Temporal Ctx | |
| AD 3 Hippo | 8.7 | AD 1 Occipital Ctx | 18.2 |
| AD 4 Hippo | 0.0 | AD 2 Occipital Ctx | 0.0 |
| | | (Missing) | |
| AD 5 hippo | 32.1 | AD 3 Occipital Ctx | 0.0 |
| AD 6 Hippo | 95.3 | AD 4 Occipital Ctx | 8.5 |
| Control 2 Hippo | 0.0 | AD 5 Occipital Ctx | 6.6 |
| Control 4 Hippo | 9.4 | AD 6 Occipital Ctx | 22.8 |
| Control (Path) 3 Hippo | 21.6 | Control 1 Occipital Ctx | 0.0 |
| AD 1 Temporal Ctx | 8.2 | Control 2 Occipital Ctx | 36.3 |
| AD 2 Temporal Ctx | 4.3 | Control 3 Occipital Ctx | 0.0 |
| AD 3 Temporal Ctx | 0.0 | Control 4 Occipital Ctx | 0.0 |
| AD 4 Temporal Ctx | 8.2 | Control (Path) 1 | 100.0 |
| | | Occipital Ctx | |
| AD 5 Inf Temporal Ctx | 32.5 | Control (Path) 2 | 15.3 |
| | | Occipital Ctx | |
| AD 5 SupTemporal Ctx | 15.2 | Control (Path) 3 | 0.0 |
| | | Occipital Ctx | |
| AD 6 Inf Temporal Ctx | 66.9 | Control (Path) 4 | 25.5 |
| | | Occipital Ctx | |
| AD 6 Sup Temporal Ctx | 67.8 | Control 1 Parietal Ctx | 0.0 |
| Control 1 Temporal Ctx | 0.0 | Control 2 Parietal Ctx | 5.6 |
| Control 2 Temporal Ctx | 18.0 | Control 3 Parietal Ctx | 20.4 |
| Control 3 Temporal Ctx | 8.1 | Control (Path) 1 | 37.9 |
| | | Parietal Ctx | |
| Control 4 Temporal Ctx | 17.7 | Control (Path) 2 | 7.5 |
| | | Parietal Ctx | |
| Control (Path) 1 | 14.8 | Control (Path) 3 | 0.0 |
| Temporal Ctx | | Parietal Ctx | |
| Control (Path) 2 | 34.2 | Control (Path) 4 | 43.8 |
| Temporal Ctx | | Parietal Ctx | |

TABLE AC
Panel 1.3D

| | Rel. Exp.(%) Ag3230, Run | | Rel. Exp.(%) Ag3230, Run |
|---|---|---|---|
| Tissue Name | 165524130 | Tissue Name | 165524130 |
| Liver adenocarcinoma | 0.0 | Kidney (fetal) | 0.0 |
| Pancreas | 0.0 | Renal ca. 786-0 | 9.8 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 11.1 | Renal ca. TK-10 | 0.0 |
| Brain (fetal) | 0.0 | Liver | 0.0 |
| Brain (whole) | 20.0 | Liver (fetal) | 0.0 |
| Brain (amygdala) | 33.9 | Liver ca. (hepatoblast) | 18.0 |
| | | HepG2 | |
| Brain (cerebellum) | 0.0 | Lung | 0.0 |
| Brain (hippocampus) | 16.4 | Lung (fetal) | 0.0 |
| Brain (substantia nigra) | 14.1 | Lung ca. (small cell) LX-1 | 17.9 |
| Brain (thalamus) | 0.0 | Lung ca. (small cell) | 0.0 |
| | | NCI-H69 | |
| Cerebral Cortex | 22.2 | Lung ca. (s. cell var.) | 0.0 |
| | | SHP-77 | |
| Spinal cord | 0.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW900 | 0.0 |
| astrocytoma SNB-75 | 13.8 | Lung ca. (squam.) NCI- | 0.0 |
| | | H596 | |
| glioma SNB-19 | 0.0 | Mammary gland | 0.0 |
| glioma U251 | 100.0 | Breast ca.* (pl.ef) MCF-7 | 12.7 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 39.5 | Ovarian ca. OVCAR-8 | 0.0 |
| Colorectal | 43.2 | Ovarian ca. IGROV-1 | 0.0 |
| Stomach | 43.5 | Ovarian ca.* (ascites) | 36.6 |
| | | SK-OV-3 | |
| Small intestine | 38.7 | Uterus | 57.8 |
| Colon ca. SW480 | 0.0 | Placenta | 0.0 |
| Colon ca.* SW620(SW480 | 0.0 | Prostate | 38.2 |
| met) | | | |
| Colon ca. HT29 | 0.0 | Prostate ca.* (bone | 0.0 |
| | | met)PC-3 | |
| Colon ca. HCT-116 | 0.0 | Testis | 17.9 |
| Colon ca. CaCo-2 | 13.4 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 14.3 | Melanoma UACC-62 | 0.0 |
| Gastric ca.* (liver met) | 79.6 | Melanoma M14 | 18.6 |
| NCI-N87 | | | |
| Bladder | 26.8 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE AD
Panel 2.2

| | Rel. Exp.(%) Ag3230, | | Rel. Exp.(%) Ag3230, |
|---|---|---|---|
| Tissue Name | Run 174442812 | Tissue Name | Run 174442812 |
| Normal Colon | 18.2 | Kidney Margin (OD04348) | 38.4 |
| Colon cancer (OD06064) | 0.0 | Kidney malignant cancer | 23.2 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 0.0 |
| | | 01) | |
| Colon Margin (OD06159) | 0.0 | Kidney Margin (OD04450- | 0.0 |
| | | 03) | |
| Colon cancer (OD06297-04) | 0.0 | Kidney Cancer 8120613 | 22.7 |
| Colon Margin (OD06297-05) | 17.9 | Kidney Margin 8120614 | 0.0 |
| CC Gr.2 ascend colon | 0.0 | Kidney Cancer 9010320 | 0.0 |
| (ODO3921) | | | |
| CC Margin (ODO3921) | 9.0 | Kidney Margin 9010321 | 0.0 |
| Colon cancer metastasis | 0.0 | Kidney Cancer 8120607 | 0.0 |
| (OD06104) | | | |
| Lung Margin (OD06104) | 0.0 | Kidney Margin 8120608 | 20.3 |
| Colon mets to lung | 41.5 | Normal Uterus | 12.6 |
| (OD04451-01) | | | |
| Lung Margin (OD04451-02) | 19.9 | Uterine Cancer 064011 | 0.0 |
| Normal Prostate | 0.0 | Normal Thyroid | 0.0 |
| Prostate Cancer (OD04410) | 12.6 | Thyroid Cancer 064010 | 0.0 |
| Prostate Margin (OD04410) | 20.0 | Thyroid Cancer A302152 | 0.0 |
| Normal Ovary | 0.0 | Thyroid Margin A302153 | 0.0 |
| Ovarian cancer (OD06283- | 0.0 | Normal Breast | 44.4 |
| 03) | | | |
| Ovarian Margin (OD06283- | 0.0 | Breast Cancer (OD04566) | 0.0 |
| 07) | | | |
| Ovarian Cancer 064008 | 41.8 | Breast Cancer 1024 | 18.9 |
| Ovarian cancer (OD06145) | 0.0 | Breast Cancer (OD04590- | 0.0 |
| | | 01) | |
| Ovarian Margin (OD06145) | 4.7 | Breast Cancer Mets | 0.0 |
| | | (OD04590-03) | |
| Ovarian cancer (OD06455- | 0.0 | Breast Cancer Metastasis | 38.2 |
| 03) | | (OD04655-05) | |
| Ovarian Margin (OD06455- | 0.0 | Breast Cancer 064006 | 15.7 |
| 07) | | | |
| Normal Lung | 32.1 | Breast Cancer 9100266 | 0.0 |
| Invasive poor diff. lung | 0.0 | Breast Margin 9100265 | 0.0 |
| adeno (ODO4945-01 | | | |
| Lung Margin (ODO4945-03) | 0.0 | Breast Cancer A209073 | 0.0 |
| Lung Malignant Cancer | 0.0 | Breast Margin A2090734 | 20.4 |
| (OD03126) | | | |
| Lung Margin (OD03126) | 0.0 | Breast cancer (OD06083) | 39.0 |
| Lung Cancer (OD05014A) | 0.0 | Breast cancer node | 45.1 |
| | | metastasis (OD06083) | |
| Lung Margin (OD05014B) | 100.0 | Normal Liver | 6.0 |
| Lung cancer (OD06081) | 0.0 | Liver Cancer 1026 | 0.0 |
| Lung Margin (OD06081) | 0.0 | Liver Cancer 1025 | 27.9 |
| Lung Cancer (OD04237-01) | 0.0 | Liver Cancer 6004-T | 20.0 |
| Lung Margin (OD04237-02) | 0.0 | Liver Tissue 6004-N | 0.0 |
| Ocular Melanoma Metastasis | 12.1 | Liver Cancer 6005-T | 0.0 |
| Ocular Melanoma Margin | 0.0 | Liver Tissue 6005-N | 0.0 |
| (Liver) | | | |
| Melanoma Metastasis | 18.8 | Liver Cancer 064003 | 19.2 |
| Melanoma Margin (Lung) | 0.0 | Normal Bladder | 0.0 |
| Normal Kidney | 20.4 | Bladder Cancer 1023 | 0.0 |
| Kidney Ca, Nuclear grade 2 | 82.4 | Bladder Cancer A302173 | 0.0 |
| (OD04338) | | | |
| Kidney Margin (OD04338) | 9.0 | Normal Stomach | 44.4 |
| Kidney Ca Nuclear grade ½ | 53.2 | Gastric Cancer 9060397 | 0.0 |
| (OD04339) | | | |
| Kidney Margin (OD04339) | 18.3 | Stomach Margin 9060396 | 18.7 |
| Kidney Ca, Clear cell type | 38.2 | Gastric Cancer 9060395 | 33.0 |
| (OD04340) | | | |
| Kidney Margin (OD04340) | 0.0 | Stomach Margin 9060394 | 21.0 |
| Kidney Ca, Nuclear grade 3 | 0.0 | Gastric Cancer 064005 | 0.0 |
| (OD04348) | | | |

TABLE AE
Panel 4D

| | Rel. Exp.(%) Ag3230, | | Rel. Exp.(%) Ag3230, |
|---|---|---|---|
| Tissue Name | Run 164532020 | Tissue Name | Run 164532020 |
| Secondary Th1 act | 0.0 | HUVEC IL-1beta | 7.9 |
| Secondary Th2 act | 0.0 | HUVEC IFN gamma | 9.2 |
| Secondary Tr1 act | 11.1 | HUVEC TNF alpha + IFN | 0.0 |
| | | gamma | |
| Secondary Th1 rest | 0.0 | HUVEC TNF alpha + IL4 | 0.0 |
| Secondary Th2 rest | 13.9 | HUVEC IL-11 | 4.0 |
| Secondary Tr1 rest | 12.2 | Lung Microvascular EC none | 15.2 |
| Primary Th1 act | 7.1 | Lung Microvascular EC | 15.2 |
| | | TNF alpha + IL-1beta | |
| Primary Th2 act | 17.7 | Microvascular Dermal EC none | 9.2 |
| Primary Tr1 act | 0.0 | Microsvasular Dermal EC | 7.2 |
| | | TNF alpha + IL-1beta | |
| Primary Th1 rest | 57.0 | Bronchial epithelium TNF alpha + IL1beta | 15.9 |
| Primary Th2 rest | 11.5 | Small airway epithelium none | 22.4 |
| Primary Tr1 rest | 7.9 | Small airway epithelium | 100.0 |
| | | TNF alpha + IL-1beta | |
| CD45RA CD4 lymphocyte | 1.9 | Coronery artery SMC rest | 0.0 |
| act | | | |
| CD45RO CD4 lymphocyte | 25.2 | Coronery artery SMC TNF alpha + IL-1beta | 0.0 |
| act | | | |
| CD8 lymphocyte act | 7.1 | Astrocytes rest | 0.0 |
| Secondary CD8 | 53.6 | Astrocytes TNF alpha + IL-1beta | 0.0 |
| lymphocyte rest | | | |
| Secondary CD8 | 0.0 | KU-812 (Basophil) rest | 0.0 |
| lymphocyte act | | | |
| CD4 lymphocyte none | 41.8 | KU-812 (Basophil) | 58.2 |
| | | PMA/ionomycin | |
| 2ry Th1/Th2/Tr1_anti- | 7.0 | CCD1106 (Keratinocytes) none | 0.0 |
| CD95 CH11 | | | |
| LAK cells rest | 24.8 | CCD1106 (Keratinocytes) | 0.0 |
| | | TNF alpha + IL-1beta | |
| LAK cells IL-2 | 32.5 | Liver cirrhosis | 51.1 |
| LAK cells IL-2 + IL-12 | 22.2 | Lupus kidney | 0.0 |
| LAK cells IL-2 + IFN | 27.4 | NCI-H292 none | 0.0 |
| gamma | | | |
| LAK cells IL-2 + IL-18 | 15.3 | NCI-H292 IL-4 | 31.9 |
| LAK cells | 0.0 | NCI-H292 IL-9 | 12.3 |
| PMA/ionomycin | | | |
| NK Cells IL-2 rest | 12.8 | NCI-H292 IL-13 | 0.0 |
| Two Way MLR 3 day | 28.9 | NCI-H292 IFN gamma | 0.0 |
| Two Way MLR 5 day | 0.0 | HPAEC none | 0.0 |
| Two Way MLR 7 day | 27.5 | HPAEC TNF alpha + IL-1beta | 3.2 |
| PBMC rest | 9.4 | Lung fibroblast none | 20.9 |
| PBMC PWM | 21.8 | Lung fibroblast TNF alpha + IL- | 14.9 |
| | | 1beta | |
| PBMC PHA-L | 7.9 | Lung fibroblast IL-4 | 13.2 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 25.9 | Lung fibroblast IFN gamma | 0.0 |
| B lymphocytes CD40L | 30.8 | Dermal fibroblast CCD1070 rest | 0.0 |
| and IL-4 | | | |
| EOL-1 dbcAMP | 0.0 | Dermal fibroblast CCD1070 | 0.0 |
| | | TNF alpha | |
| EOL-1 dbcAMP | 0.0 | Dermal fibroblast CCD1070 IL- | 0.0 |
| PMA/ionomycin | | 1beta | |
| Dendritic cells none | 59.5 | Dermal fibroblast IFN gamma | 0.0 |
| Dendritic cells LPS | 0.0 | Dermal fibroblast IL-4 | 7.7 |
| Dendritic cells anti-CD40 | 54.3 | IBD Colitis 2 | 0.0 |
| Monocytes rest | 49.7 | IBD Crohn's | 7.1 |
| Monocytes LPS | 8.7 | Colon | 14.4 |
| Macrophages rest | 21.6 | Lung | 16.2 |
| Macrophages LPS | 8.0 | Thymus | 92.7 |
| HUVEC none | 0.0 | Kidney | 0.0 |
| HUVEC starved | 0.0 | | |

CNS_neurodegeneration_v1.0 Summary: Ag3230 This panel does not show differential expression of the CG57413-01 gene in Alzheimer's disease. However, this expression profile confirms the presence of this gene in the brain. This gene encodes a homolog of an androgen regulated short chain dehydrogenase. Members of this family are known to function in the processing of hormones in the brain. Brain hormone regulation mediates numerous clinically significant conditions, including psychiatric disorders such as anxiety, overeating and memory disorders. Therefore, agents that modulate the activity of this gene product have potential utility in the treatment of these disorders. In addition, steroid treatment is used in a number of clinical conditions including Alzheimer's disease (estrogen), menopause associated symptoms (estrogen), multiple sclerosis (glucocorticoids), and spinal cord injury (methylprednisolone). Treatment with an antagonist of this gene product, or reduction of the levels of this gene product could slow steroid degredation and lower the necessary amount given for therapeutic effect, thus reducing peripheral side effects (Biswas and Russell, J Biol Chem 1997 Jun. 20; 272 (25):15959-66; Matsumoto et al., Spine 2001 Feb. 15; 26 (4):426-30; Holinka, Ann N Y Acad Sci 2001 September; 943:89-108; Burkman et al., Am J Obstet Gynecol 2001 August; 185 (2 Suppl):S13-23; Gaillard et al., Neuroreport 2001 Jul. 20; 12 (10):2189-93; Penning et al., Biochem J 2000 Oct. 1; 351 (Pt 1):67-77).

Panel 1.3D Summary: Ag3230 Expression of the CG57413-01 gene is restricted to a sample derived from a brain cancer cell line (CT=34.8). Thus, expression of this gene could be used to differentiate between this sample and other samples on this panel and as a marker to detect the presence of brain cancer. Furthermore, therapeutic modulation of the expression or function of this gene may be effective in the treatment of brain cancer.

Panel 2.2 Summary: Ag3230 Expression of the CG57413-01 gene is restricted to a sample derived from a normal lung tissue (CT=34.95). Thus, expression of this gene could be used to differentiate between this sample and other samples on this panel and as a marker of normal lung tissue.

Panel 4D Summary: Ag3230 Highest expression of the CG57413-01 gene is seen in Small airway epithelium stimulated with TNFalpha+IL-1beta (CT=33.4). Significant expression is also seen in the basophil cell line KU-812 stimulated with PMA/ionomycin. This cell line constitutes a reasonable model for the inflammatory cells that take part in various inflammatory lung diseases. This expression profile in combination with the exclusive expression in normal lung seen in Panel 2D suggests that the protein encoded by this gene may be involved in the proliferation or activation of airway epithelium. Therefore, therapuetics designed with the protein encoded by this transcript could be important in the treatment of pathological and inflammatory lung disorders, including emphysema, allergies, asthma and COPD.

Panel 5 Islet Summary: Ag3230 Expression of the CG57413-01 gene is low/undetectable in all samples on this panel (CTs>35).

B. NOV2a: Insulysin

Expression of gene CG57433-01 was assessed using the primer-probe set Ag3225, described in Table BA. Results of the RTQ-PCR runs are shown in Tables BB, BC and BD.

TABLE BA

Probe Name Ag3225

| | | | |
|---|---|---|---|
| | | | Start |
| Pri- | | | Posi- |
| mers | Sequences | Length | tion |

| | | | |
|---|---|---|---|
| For- | 5′-gggtgtttaggattccctgtag-3′ | 22 | 677 |
| ward | (SEQ ID NO:36) | | |
| Probe | TET-5′- | 26 | 645 |
| | ccaattgaaagagtctccaggcatca-3′- | | |
| | TAMRA (SEQ ID NO:37) | | |
| Re- | 5′-tgcagttgattcagaacatgag-3′ | 22 | 609 |
| verse | (SEQ ID NO:38) | | |

TABLE BB
Panel 1.3D

| | Rel. Exp.(%) Ag3225, Run | | Rel. Exp.(%) Ag3225, Run |
|---|---|---|---|
| Tissue Name | 165524115 | Tissue Name | 165524115 |
| Liver adenocarcinoma | 29.9 | Kidney (fetal) | 6.7 |
| Pancreas | 7.2 | Renal ca. 786-0 | 27.2 |
| Pancreatic ca. CAPAN 2 | 31.0 | Renal ca. A498 | 23.5 |
| Adrenal gland | 8.7 | Renal ca. RXF 393 | 23.3 |
| Thyroid | 10.2 | Renal ca. ACHN | 20.9 |
| Salivary gland | 7.1 | Renal ca. UO-31 | 28.5 |
| Pituitary gland | 10.3 | Renal ca. TK-10 | 7.1 |
| Brain (fetal) | 12.2 | Liver | 3.3 |
| Brain (whole) | 24.1 | Liver (fetal) | 22.2 |
| Brain (amygdala) | 7.7 | Liver ca. (hepatoblast) | 47.0 |
| | | HepG2 | |
| Brain (cerebellum) | 21.5 | Lung | 8.2 |
| Brain (hippocampus) | 11.0 | Lung (fetal) | 2.7 |
| Brain (substantia nigra) | 7.2 | Lung ca. (small cell) LX-1 | 37.1 |
| Brain (thalamus) | 20.6 | Lung ca. (small cell) | 5.0 |
| | | NCI-H69 | |
| Cerebral Cortex | 13.5 | Lung ca. (s. cell var.) | 16.6 |
| | | SHP-77 | |
| Spinal cord | 14.3 | Lung ca. (large cell)NCI- | 100.0 |
| | | H460 | |
| glio/astro U87-MG | 30.1 | Lung ca. (non-sm. cell) | 35.6 |
| | | A549 | |
| glio/astro U-118-MG | 50.3 | Lung ca. (non-s. cell) | 23.3 |
| | | NCI-H23 | |
| astrocytoma SW1783 | 31.0 | Lung ca. (non-s. cell) | 30.1 |
| | | HOP-62 | |
| neuro*; met SK-N-AS | 31.2 | Lung ca. (non-s. cl) NCI- | 13.1 |
| | | H522 | |
| astrocytoma SF-539 | 25.5 | Lung ca. (squam.) SW900 | 19.9 |
| astrocytoma SNB-75 | 34.6 | Lung ca. (squam.) NCI- | 14.2 |
| | | H596 | |
| glioma SNB-19 | 28.7 | Mammary gland | 12.7 |
| glioma U251 | 92.7 | Breast ca.* (pl.ef) MCF-7 | 31.0 |
| glioma SF-295 | 24.7 | Breast ca.* (pl.ef) MDA- | 62.9 |
| | | MB-231 | |
| Heart (fetal) | 5.1 | Breast ca.* (pl.ef) T47D | 13.9 |
| Heart | 14.2 | Breast ca. BT-549 | 61.1 |
| Skeletal muscle (fetal) | 2.2 | Breast ca. MDA-N | 5.7 |
| Skeletal muscle | 90.1 | Ovary | 2.8 |
| Bone marrow | 7.5 | Ovarian ca. OVCAR-3 | 27.2 |
| Thymus | 7.0 | Ovarian ca. OVCAR-4 | 23.8 |
| Spleen | 12.2 | Ovarian ca. OVCAR-5 | 18.0 |
| Lymph node | 20.3 | Ovarian ca. OVCAR-8 | 8.6 |
| Colorectal | 7.5 | Ovarian ca. IGROV-1 | 10.2 |
| Stomach | 17.6 | Ovarian ca.* (ascites) | 20.0 |
| | | SK-OV-3 | |
| Small intestine | 17.4 | Uterus | 17.8 |
| Colon ca. SW480 | 18.9 | Placenta | 7.1 |
| Colon ca.* SW620(SW480 | 25.7 | Prostate | 7.9 |
| met) | | | |
| Colon ca. HT29 | 9.7 | Prostate ca.* (bone | 16.6 |
| | | met)PC-3 | |
| Colon ca. HCT-116 | 30.6 | Testis | 10.2 |
| Colon ca. CaCo-2 | 29.9 | Melanoma Hs688(A).T | 9.6 |
| Colon ca. | 24.5 | Melanoma* (met) | 10.9 |
| tissue(ODO3866) | | Hs688(B).T | |
| Colon ca. HCC-2998 | 14.3 | Melanoma UACC-62 | 8.0 |
| Gastric ca.* (liver met) | 36.1 | Melanoma M14 | 47.0 |
| NCI-N87 | | | |
| Bladder | 17.4 | Melanoma LOX IMVI | 3.4 |
| Trachea | 7.5 | Melanoma* (met) SK- | 9.2 |
| | | MEL-5 | |
| Kidney | 7.0 | Adipose | 9.2 |

TABLE BC
Panel 5 Islet

| | Rel. Exp.(%) | | Rel. Exp.(%) |
|---|---|---|---|
| | Ag3225, Run | | Ag3225, Run |
| Tissue Name | 223846465 | Tissue Name | 223846465 |
| 97457_Patient- | 12.2 | 94709_Donor 2 AM - A_adipose | 32.5 |
| 02go_adipose | | | |
| 97476_Patient-07sk_skeletal | 13.6 | 94710_Donor 2 AM - B_adipose | 16.5 |
| 97477_Patient-07ut_uterus | 10.5 | 94711_Donor 2 AM - C_adipose | 15.8 |
| 97478_Patient- | 15.5 | 94712_Donor 2 AD - A_adipose | 37.9 |
| 07pl_placenta | | | |
| 99167_Bayer Patient 1 | 9.7 | 94713_Donor 2 AD - B_adipose | 37.4 |
| 97482_Patient-08ut_uterus | 7.8 | 94714_Donor 2 AD - C_adipose | 41.5 |
| 97483_Patient- | 13.3 | 94742_Donor 3 U - A_Mesenchymal | 5.7 |
| 08pl_placenta | | Stem Cells | |
| 97486_Patient-09sk_skeletal | 4.0 | 94743_Donor 3 U - B_Mesenchymal | 25.7 |
| muscle | | Stem Cells | |
| 97487_Patient-09ut_uterus | 18.4 | 94730_Donor 3 AM - A_adipose | 42.0 |
| 97488_Patient- | 6.9 | 94731_Donor 3 AM - B_adipose | 16.4 |
| 09pl_placenta | | | |
| 97492_Patient-10ut_uterus | 28.5 | 94732_Donor 3 AM - C_adipose | 26.4 |
| 97493_Patient- | 23.0 | 94733_Donor 3 AD - A_adipose | 59.5 |
| 10pl_placenta | | | |
| 97495_Patient- | 8.9 | 94734_Donor 3 AD - B_adipose | 11.7 |
| 11go_adipose | | | |
| 97496_Patient-11sk_skeletal | 20.7 | 94735_Donor 3 AD - C_adipose | 35.4 |
| muscle | | | |
| 97497_Patient-11ut_uterus | 23.5 | 77138_Liver_HepG2untreated | 100.0 |
| 97498_Patient- | 17.0 | 73556_Heart_Cardiac stromal cells | 10.8 |
| 11pl_placenta | | (primary) | |
| 97500_Patient- | 18.3 | 81735_Small Intestine | 26.8 |
| 12go_adipose | | | |
| 97501_Patient-12sk_skeletal | 49.3 | 72409_Kidney_Proximal Convoluted | 12.1 |
| muscle | | Tubule | |
| 97502_Patient-12ut_uterus | 29.7 | 82685_Small intestine_Duodenum | 10.4 |
| 97503_Patient- | 12.9 | 90650_Adrenal_Adrenocortical | 9.8 |
| 12pl_placenta | | adenoma | |
| 94721_Donor 2 U - | 14.3 | 72410_Kidney_HRCE | 38.4 |
| A_Mesenchymal Stem Cells | | | |
| 94722_Donor 2 U - | 12.3 | 72411_Kidney_HRE | 32.3 |
| B_Mesenchymal Stem Cells | | | |
| 94723_Donor 2 U - | 23.5 | 73139_Uterus_Uterine smooth | 11.0 |
| C_Mesenchymal Stem Cells | | muscle cells | |

TABLE BD
Panel 5D

| | Rel. Exp.(%) | | Rel. Exp.(%) |
|---|---|---|---|
| | Ag3225, Run | | Ag3225, Run |
| Tissue Name | 169271478 | Tissue Name | 169271478 |
| 97457_Patient- | 9.7 | 94709_Donor 2 AM - A_adipose | 26.2 |
| 02go_adipose | | | |
| 97476_Patient-07sk_skeletal | 7.1 | 94710_Donor 2 AM - B_adipose | 19.2 |
| muscle | | | |
| 97477_Patient-07ut_uterus | 9.7 | 94711_Donor 2 AM - C_adipose | 18.4 |
| 97478_Patient- | 13.9 | 94712_Donor 2 AD - A_adipose | 35.1 |
| 07pl_placenta | | | |
| 97481_Patient-08sk_skeletal | 7.7 | 94713_Donor 2 AD - B_adipose | 37.9 |
| muscle | | | |
| 97482_Patient-08ut_uterus | 10.2 | 94714_Donor 2 AD - C_adipose | 38.4 |
| 97483_Patient- | 7.9 | 94742_Donor 3 U - A_Mesenchymal | 18.9 |
| 08pl_placenta | | Stem Cells | |
| 97486_Patient-09sk_skeletal | 7.7 | 94743_Donor 3 U - B_Mesenchymal | 25.7 |
| muscle | | Stem Cells | |
| 97487_Patient-09ut_uterus | 12.4 | 94730_Donor 3 AM - A_adipose | 40.1 |
| 97488_Patient- | 8.4 | 94731_Donor 3 AM - B_adipose | 20.7 |
| 09pl_placenta | | | |
| 97492_Patient-10ut_uterus | 16.0 | 94732_Donor 3 AM - C_adipose | 30.1 |
| 97493_Patient- | 24.5 | 94733_Donor 3 AD - A_adipose | 43.2 |
| 10pl_placenta | | | |
| 97495_Patient- | 6.7 | 94734_Donor 3 AD - B_adipose | 27.2 |
| 11go_adipose | | | |
| 97496_Patient-11sk_skeletal | 22.5 | 94735_Donor 3 AD - C_adipose | 37.9 |
| muscle | | | |
| 97497_Patient-11ut_uterus | 22.4 | 77138_Liver_HepG2untreated | 100.0 |
| 97498_Patient- | 8.2 | 73556_Heart_Cardiac stromal cells | 9.7 |
| 11pl_placenta | | (primary) | |
| 97500_Patient- | 9.3 | 81735_Small Intestine | 16.5 |
| 12go_adipose | | | |
| 97501_Patient-12sk_skeletal | 43.2 | 72409_Kidney_Proximal Convoluted | 10.2 |
| muscle | | Tubule | |
| 97502_Patient-12ut_uterus | 25.2 | 82685_Small intestine_Duodenum | 13.6 |
| 97503_Patient- | 9.6 | 90650_Adrenal_Adrenocortical | 8.5 |
| 12pl_placenta | | adenoma | |
| 94721_Donor 2 U - | 15.6 | 72410_Kidney_HRCE | 26.8 |
| A_Mesenchymal Stem Cells | | | |
| 94722_Donor 2 U - | 12.6 | 72411_Kidney_HRE | 27.2 |
| B_Mesenchymal Stem Cells | | | |
| 94723_Donor 2 U - | 13.3 | 73139_Uterus_Uterine smooth | 11.0 |
| C_Mesenchymal Stem Cells | | muscle cells | |

Panel 1.3D Summary: 3225 The expression of the CG57433-01 gene appears to be highest in a sample derived from a lung cancer cell line (H460 (CT=29.5). In addition, there appears to be substantial expression in other samples derived from lung cancer cell lines, brain cancer cell lines, kidney cancer cell lines, colon cancer cell lines and breast cancer cell lines. Of note, is the apparent overexpression of this gene in skeletal muscle (CT=30) in comparison to fetal skeletal muscle (CT=35). Thus, the expression of this gene could be used to distinguish H460 cells from other samples in this panel. In addition, the expression of this gene could be used to distinguish adult skeletal muscle and fetal skeletal muscle. Moreover, therapeutic modulation of this gene, through the use of small molecule drugs, protein therapeutics, or antibodies could be of benefit in the treatment of lung, brain, kidney, colon or breast cancer.

Among metabolic tissues, this gene has low levels of expression in pancreas, adrenal, thyroid, pituitary, heart, liver and adipose. Inhibition of this insulin-degrading enzyme may increase insulin signalling and action and thus be a treatment for the prevention and treatment of Type 2 diabetes. This gene product may also be a small molecule target for the treatment of metabolic and endocrine disease, including thyroidopathies, Type 1 diabetes, and obesity.

This gene is also expressed throughout the brain at low levels, indicating a role in CNS processes. Insulysin (also known as Insulin-degrading enyzme) has been shown to rapidly remove the beta-amyloid precursor protein intracellular domain (AICD) and eliminate proteins with amyloidogenic potential, which are thought to mediate of the pathological features of Alzheimer's disease. Potentiation of this process is a likely means of addressing Alzneimer's disease pathology. Therefore, agents that increase the physiological activity of this gene product are likely to have utility as clinically therapeutic treatments for Alzheimer's disease, and potentially other neurodegenerative diseases involving protein aggregation (Edbauer et al., Biol Chem 2002 Jan. 23; [epub ahead of print]; Kurochkin, Trends Biochem Sci 2001 July; 26 (7):421-5).

Panel 5 Islet and 5D Summary: Ag3225 The CG57433-01 gene is expressed at low levels in islets of Langerhans and mesenchymal stem cells that differentiate in vitro into adipocytes, chondrocytes and osteocytes. Therefore, therapeutic modulation of this enzyme may be a treatment for diseases of bone and cartilage as well as Types 1 and 2 diabetes.

C. NOV3: BCSC-1

Expression of gene CG57362-01 was assessed using the primer-probe set Ag3217, described in Table CA. Results of the RTQ-PCR runs are shown in Tables CB, CC, CD, CE and CF.

TABLE CA

Probe Name Ag3217

| | | | |
|---|---|---|---|
| | | | Start |
| Pri- | | | Posi- |
| mers | Sequences | Length | tion |

| | | | |
|---|---|---|---|
| For- | 5′-actgggaacaggcactgtct-3′ | 20 | 1327 |
| ward | (SEQ ID NO:39) | | |
| Probe | TET- | 23 | 1347 |
| | 5′-cagcagaactgtccagcccatgg-3′- | | |
| | TAMRA (SEQ ID NO:40) | | |
| Re- | 5′-gggtctgtcagagcatcagtac-3′ | 22 | 1395 |
| verse | (SEQ ID NO:41) | | |

TABLE CB
CNS_neurodegeneration_v1.0

| | Rel. Exp.(%) | Rel. Exp.(%) | | Rel. Exp.(%) | Rel. Exp.(%) |
|---|---|---|---|---|---|
| | Ag3217, Run | Ag3217, Run | | Ag3217, Run | Ag3217, Run |
| Tissue Name | 209861783 | 224079666 | Tissue Name | 209861783 | 224079666 |
| AD 1 Hippo | 18.3 | 24.8 | Control (Path) | 15.6 | 17.7 |
| | | | 3 Temporal Ctx | | |
| AD 2 Hippo | 30.4 | 42.6 | Control (Path) | 72.2 | 81.8 |
| | | | 4 Temporal Ctx | | |
| AD 3 Hippo | 20.2 | 18.3 | AD 1 Occipital | 25.2 | 32.5 |
| | | | Ctx | | |
| AD 4 Hippo | 21.3 | 22.2 | AD 2 Occipital | 0.0 | 0.0 |
| | | | Ctx (Missing) | | |
| AD 5 Hippo | 82.9 | 78.5 | AD 3 Occipital | 7.2 | 7.4 |
| | | | Ctx | | |
| AD 6 Hippo | 29.9 | 33.2 | AD 4 Occipital | 52.1 | 36.6 |
| | | | Ctx | | |
| Control 2 | 43.2 | 45.1 | AD 5 Occipital | 46.3 | 38.4 |
| Hippo | | | Ctx | | |
| Control 4 | 26.2 | 17.7 | AD 6 Occipital | 17.2 | 17.9 |
| Hippo | | | Ctx | | |
| Control (Path) | 24.3 | 12.8 | Control 1 | 5.1 | 8.0 |
| 3 Hippo | | | Occipital Ctx | | |
| AD 1 | 12.6 | 15.3 | Control 2 | 51.8 | 75.8 |
| Temporal Ctx | | | Occipital Ctx | | |
| AD 2 | 51.8 | 33.4 | Control 3 | 71.7 | 85.9 |
| Temporal Ctx | | | Occipital Ctx | | |
| AD 3 | 12.3 | 13.3 | Control 4 | 14.4 | 17.6 |
| Temporal Ctx | | | Occipital Ctx | | |
| AD 4 | 45.4 | 46.7 | Control (Path) | 100.0 | 100.0 |
| Temporal Ctx | | | 1 Occipital Ctx | | |
| AD 5 Inf | 56.6 | 51.4 | Control (Path) | 46.3 | 53.2 |
| Temporal Ctx | | | 2 Occipital Ctx | | |
| AD 5 Sup | 43.8 | 33.2 | Control (Path) | 5.9 | 6.3 |
| Temporal Ctx | | | 3 Occipital Ctx | | |
| AD 6 Inf | 40.3 | 33.7 | Control (Path) | 50.7 | 58.6 |
| Temporal Ctx | | | 4 Occipital Ctx | | |
| AD 6 Sup | 38.4 | 46.3 | Control 1 | 10.6 | 10.4 |
| Temporal Ctx | | | Parietal Ctx | | |
| Control 1 | 12.9 | 11.0 | Control 2 | 34.4 | 43.2 |
| Temporal Ctx | | | Parietal Ctx | | |
| Control 2 | 45.7 | 41.8 | Control 3 | 49.7 | 37.1 |
| Temporal Ctx | | | Parietal Ctx | | |
| Control 3 | 57.4 | 71.7 | Control (Path) | 82.4 | 86.5 |
| Temporal Ctx | | | 1 Parietal Ctx | | |
| Control 3 | 42.3 | 37.1 | Control (Path) | 55.5 | 63.7 |
| Temporal Ctx | | | 2 Parietal Ctx | | |
| Control (Path) | 87.1 | 99.3 | Control (Path) | 12.9 | 9.3 |
| 1 Temporal | | | 3 Parietal Ctx | | |
| Ctx | | | | | |
| Control (Path) | 92.0 | 97.3 | Control (Path) | 90.1 | 95.9 |
| 2 Temporal | | | 4 Parietal Ctx | | |
| Ctx | | | | | |

TABLE CC
Panel 1.3D

| | Rel. Exp.(%) Ag3217, Run | | Rel. Exp.(%) Ag3217, Run |
|---|---|---|---|
| Tissue Name | 168012860 | Tissue Name | 168012860 |
| Liver adenocarcinoma | 3.9 | Kidney (fetal) | 2.6 |
| Pancreas | 8.1 | Renal ca. 786-0 | 0.4 |
| Pancreatic ca. CAPAN 2 | 0.0 | Renal ca. A498 | 0.0 |
| Adrenal gland | 24.3 | Renal ca. RXF 393 | 0.0 |
| Thyroid | 0.0 | Renal ca. ACHN | 0.0 |
| Salivary gland | 5.4 | Renal ca. UO-31 | 0.1 |
| Pituitary gland | 12.9 | Renal ca. TK-10 | 1.2 |
| Brain (fetal) | 10.2 | Liver | 0.9 |
| Brain (whole) | 31.6 | Liver (fetal) | 0.0 |
| Brain (amygdala) | 72.2 | Liver ca. (hepatoblast) | 0.0 |
| | | HepG2 | |
| Brain (cerebellum) | 21.5 | Lung | 0.0 |
| Brain (hippocampus) | 39.5 | Lung (fetal) | 1.4 |
| Brain (substantia nigra) | 11.3 | Lung ca. (small cell) LX-1 | 1.7 |
| Brain (thalamus) | 31.6 | Lung ca. (small cell) | 6.6 |
| | | NCI-H69 | |
| Cerebral Cortex | 39.8 | Lung ca. (s. cell var.) | 27.7 |
| | | SHP-77 | |
| Spinal cord | 1.4 | Lung ca. (large cell)NCI- | 0.0 |
| | | H460 | |
| glio/astro U87-MG | 0.0 | Lung ca. (non-sm. cell) | 4.4 |
| | | A549 | |
| glio/astro U-118-MG | 0.0 | Lung ca. (non s. cell) | 0.4 |
| | | NCI-H23 | |
| astrocytoma SW1783 | 0.0 | Lung ca. (non-s. cell) | 0.0 |
| | | HOP-62 | |
| neuro*; met SK-N-AS | 0.3 | Lung ca. (non-s. cl) NCI- | 2.9 |
| | | H522 | |
| astrocytoma SF-539 | 0.1 | Lung ca. (squam.) SW900 | 1.0 |
| astrocytoma SNB-75 | 0.0 | Lung ca. (squam.) NCI- | 100.0 |
| | | H596 | |
| glioma SNB-19 | 2.4 | Mammary gland | 0.0 |
| glioma U251 | 3.4 | Breast ca.* (pl.ef) MCF-7 | 0.0 |
| glioma SF-295 | 0.8 | Breast ca.* (pl.ef) MDA- | 0.0 |
| | | MB-231 | |
| Heart (fetal) | 0.0 | Breast ca.* (pl.ef) T47D | 4.6 |
| Heart | 0.0 | Breast ca. BT-549 | 0.0 |
| Skeletal muscle (fetal) | 1.1 | Breast ca. MDA-N | 0.2 |
| Skeletal muscle | 0.0 | Ovary | 1.4 |
| Bone marrow | 1.4 | Ovarian ca. OVCAR-3 | 0.0 |
| Thymus | 0.0 | Ovarian ca. OVCAR-4 | 0.8 |
| Spleen | 0.4 | Ovarian ca. OVCAR-5 | 3.2 |
| Lymph node | 0.0 | Ovarian ca. OVCAR-8 | 0.8 |
| Colorectal | 2.6 | Ovarian ca. IGROV-1 | 0.0 |
| Stomach | 3.2 | Ovarian ca.* (ascites) | 0.3 |
| | | SK-OV-3 | |
| Small intestine | 1.3 | Uterus | 0.0 |
| Colon ca. SW480 | 1.5 | Placenta | 0.0 |
| Colon ca.* SW620(SW480 | 11.9 | Prostate | 1.6 |
| met) | | | |
| Colon ca. HT29 | 0.0 | Prostate ca.* (bone | 0.4 |
| | | met)PC-3 | |
| Colon ca. HCT-116 | 2.9 | Testis | 2.6 |
| Colon ca. CaCo-2 | 0.0 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.8 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

TABLE CD
Panel 2.2

| | Rel. Exp.(%) Ag3217, | | Rel. Exp.(%) Ag3217, |
|---|---|---|---|
| Tissue Name | Run 174416493 | Tissue Name | Run 174416493 |
| Normal Colon | 11.9 | Kidney Margin (OD04348) | 9.9 |
| Colon cancer (OD06064) | 0.0 | Kidney malignant cancer | 0.0 |
| | | (OD06204B) | |
| Colon Margin (OD06064) | 0.0 | Kidney normal adjacent | 0.0 |
| | | tissue (OD06204E) | |
| Colon cancer (OD06159) | 0.0 | Kidney Cancer (OD04450- | 3.9 |
| | | 01) | |
| Colon Margin (OD06159) | 6.3 | Kidney Margin (OD04450- | 0.0 |
| | | 03) | |
| Colon cancer (OD06297-04) | 0.0 | Kidney Cancer 8120613 | 0.0 |
| Colon Margin (OD06297-05) | 0.0 | Kidney Margin 8120614 | 0.0 |
| CC Gr.2 ascend colon | 0.0 | Kidney Cancer 9010320 | 47.6 |
| (ODO3921) | | | |
| CC Margin (ODO3921) | 0.0 | Kidney Margin 9010321 | 0.0 |
| Colon cancer metastasis | 0.0 | Kidney Cancer 8120607 | 3.7 |
| (OD06104) | | | |
| Lung Margin (OD06104) | 0.0 | Kidney Margin 8120608 | 0.0 |
| Colon mets to lung | 0.0 | Normal Uterus | 0.0 |
| (OD04451-01) | | | |
| Lung Margin (OD04451-02) | 0.0 | Uterine Cancer 064011 | 12.7 |
| Normal Prostate | 22.4 | Normal Thyroid | 13.6 |
| Prostate Cancer (OD04410) | 8.4 | Thyroid Cancer 064010 | 0.0 |
| Prostate Margin (OD04410) | 0.0 | Thyroid Cancer A302152 | 0.0 |
| Normal Ovary | 0.0 | Thyroid Margin A302153 | 7.6 |
| Ovarian cancer (OD06283- | 0.0 | Normal Breast | 0.0 |
| 03) | | | |
| Ovarian Margin (OD06283- | 0.0 | Breast Cancer (OD04566) | 0.0 |
| 07) | | | |
| Ovarian Cancer 064008 | 9.5 | Breast Cancer 1024 | 0.0 |
| Ovarian cancer (OD06145) | 0.0 | Breast Cancer (OD04590- | 19.3 |
| | | 01) | |
| Ovarian Margin (OD06145) | 100.0 | Breast Cancer Mets | 35.8 |
| | | (OD04590-03) | |
| Ovarian cancer (OD06455- | 0.0 | Breast Cancer Metastasis | 24.5 |
| 03) | | (OD04655-05) | |
| Ovarian Margin (OD06455- | 0.0 | Breast Cancer 064006 | 0.0 |
| 07) | | | |
| Normal Lung | 0.0 | Breast Cancer 9100266 | 10.0 |
| Invasive poor diff. lung | 0.0 | Breast Margin 9100265 | 0.0 |
| adeno (ODO4945-01) | | | |
| Lung Margin (ODO4945-03) | 0.0 | Breast Cancer A209073 | 0.0 |
| Lung Malignant Cancer | 17.4 | Breast Margin A2090734 | 9.1 |
| (OD03126) | | | |
| Lung Margin (OD03126) | 8.8 | Breast cancer (OD06083) | 0.0 |
| Lung Cancer (OD05014A) | 0.0 | Breast cancer node | 5.1 |
| | | metastasis (OD06083) | |
| Lung Margin (OD05014B) | 0.0 | Normal Liver | 6.7 |
| Lung cancer (OD06081) | 12.9 | Liver Cancer 1026 | 5.6 |
| Lung Margin (OD06081) | 0.0 | Liver Cancer 1025 | 8.5 |
| Lung Cancer (OD04237-01) | 0.0 | Liver Cancer 6004-T | 0.0 |
| Lung Margin (OD04237-02) | 0.0 | Liver Tissue 6004-N | 6.0 |
| Ocular Melanoma Metastasis | 0.0 | Liver Cancer 6005-T | 0.0 |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 8.4 |
| Normal Kidney | 0.0 | Bladder Cancer 1023 | 0.0 |
| Kidney Ca, Nuclear grade 2 | 22.1 | Bladder Cancer A302173 | 8.7 |
| (OD04338) | | | |
| Kidney Margin (OD04338) | 0.0 | Normal Stomach | 0.0 |
| Kidney Ca Nuclear grade ½ | 40.9 | Gastric Cancer 9060397 | 0.0 |
| (OD04339) | | | |
| Kidney Margin (OD04339) | 0.0 | Stomach Margin 9060396 | 12.4 |
| Kidney Ca, Clear cell type | 0.0 | Gastric Cancer 9060395 | 6.4 |
| (OD04340) | | | |
| Kidney Margin (OD04340) | 0.0 | Stomach Margin 9060394 | 9.3 |
| Kidney Ca, Nuclear grade 3 | 0.0 | Gastric Cancer 064005 | 0.0 |
| (OD04348) | | | |

TABLE CE
Panel 4D

| | Rel. Exp.(%) Ag3217, | | Rel. Exp.(%) Ag3217, |
|---|---|---|---|
| Tissue Name | Run 164682517 | Tissue Name | Run 164682517 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 6.9 | Bronchial epithelium TNF alpha + IL1beta | 4.1 |
| Primary Th2 rest | 0.0 | Small airway epithelium none | 8.2 |
| Primary Tr1 rest | 0.0 | Small airway epithelium | 0.0 |
| | | TNF alpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + IL- | 0.0 |
| act | | 1beta | |
| CD8 lymphocyte act | 3.2 | Astrocytes rest | 0.0 |
| Secondary CD8 | 0.0 | Astrocytes TNF alpha + IL-1beta | 0.0 |
| lymphocyte rest | | | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) none | 0.0 |
| CD95 CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 6.9 |
| LAK cells IL-2 + IL-12 | 0.0 | Lupus kidney | 0.0 |
| LAK cells IL-2 + IFN | 0.0 | NCI-H292 none | 5.3 |
| gamma | | | |
| LAK cells IL-2 + IL-18 | 0.0 | NCI-H292 IL-4 | 0.0 |
| LAK cells | 22.7 | NCI-H29 IL-9 | 16.8 |
| PMA/ionomycin | | | |
| NK Cells IL-2 rest | 0.0 | NCI-H292 IL-13 | 0.0 |
| Two Way MLR 3 day | 5.6 | NCI-H292 IFN gamma | 0.0 |
| Two Way MLR 5 day | 0.0 | HPAEC none | 8.5 |
| Two Way MLR 7 day | 0.0 | HPAEC TNF alpha + IL-1beta | 0.0 |
| PBMC rest | 0.0 | Lung fibroblast none | 0.0 |
| PBMC PWM | 3.8 | Lung fibroblast TNF alpha + IL- | 0.0 |
| | | 1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) ionomycin | 0.0 | Lung fibroblast IL-13 | 0.0 |
| B lymphocytes PWM | 7.2 | Lung fibroblast IFN gamma | 0.0 |
| B lymphocytes CD40L | 0.0 | Dermal fibroblast CCD1070 rest | 5.4 |
| and IL-4 | | | |
| EOL-1 dbcAMP | 5.3 | Dermal fibroblast CCD1070 | 0.0 |
| | | TNF alpha | |
| EOL-1 dbcAMP | 0.0 | Dermal fibroblast CCD1070 IL- | 0.0 |
| PMA/ionomycin | | 1beta | |
| Dendritic cells none | 0.0 | Dermal fibroblast IFN gamma | 0.0 |
| Dendritic cells LPS | 0.0 | Dermal fibroblast IL-4 | 0.0 |
| Dendritic cells anti-CD40 | 0.0 | IBD Colitis 2 | 8.3 |
| Monocytes rest | 0.0 | IBD Crohn's | 20.6 |
| Monocytes LPS | 0.0 | Colon | 100.0 |
| Macrophages rest | 0.0 | Lung | 47.6 |
| Macrophages LPS | 0.0 | Thymus | 19.3 |
| HUVEC none | 0.0 | Kidney | 0.0 |
| HUVEC starved | 0.0 | | |

TABLE CF
Panel CNS_1

| | Rel. Exp.(%) Ag3217, Run | | Rel. Exp.(%) Ag3217, Run |
|---|---|---|---|
| Tissue Name | 171694584 | Tissue Name | 171694584 |
| BA4 Control | 21.0 | BA17 PSP | 14.3 |
| BA4 Control2 | 23.3 | BA17 PSP2 | 27.2 |
| BA4 Alzheimer's2 | 9.9 | Sub Nigra Control | 16.6 |
| BA4 Parkinson's | 57.4 | Sub Nigra Control2 | 8.1 |
| BA4 Parkinson's2 | 52.5 | Sub Nigra Alzheimer's2 | 9.2 |
| BA4 Huntington's | 11.9 | Sub Nigra Parkinson's2 | 10.9 |
| BA4 | 24.1 | Sub Nigra Huntington's | 5.3 |
| Huntington's2 | | | |
| BA4 PSP | 3.6 | Sub Nigra | 17.9 |
| | | Huntington's2 | |
| BA4 PSP2 | 8.5 | SubNigra PSP2 | 1.9 |
| BA4 Depression | 20.0 | Sub Nigra Depression | 0.0 |
| BA4 Depression2 | 9.7 | Sub Nigra Depression2 | 4.1 |
| BA7 Control | 28.9 | Glob Palladus Control | 5.6 |
| BA7 Control2 | 31.0 | Glob Palladus Control2 | 4.6 |
| BA7 Alzheimer's2 | 15.7 | Glob Palladus | 5.3 |
| | | Alzheimer's | |
| BA7 Parkinson's | 31.0 | Glob Palladus | 4.7 |
| | | Alzheimer's2 | |
| BA7 Parkinson's2 | 26.2 | Glob Palladus | 29.5 |
| | | Parkinson's | |
| BA7 Huntington's | 19.9 | Glob Palladus | 8.5 |
| | | Parkinson's2 | |
| BA7 | 55.9 | Glob Palladus PSP | 2.6 |
| Huntington's2 | | | |
| BA7 PSP | 15.5 | Glob Palladus PSP2 | 4.0 |
| BA7 PSP2 | 12.3 | Glob Palladus | 4.0 |
| | | Depression | |
| BA7 Depression | 13.5 | Temp Pole Control | 13.1 |
| BA9 Control | 15.3 | Temp Pole Control2 | 31.4 |
| BA9 Control2 | 31.6 | Temp Pole Alzheimer's | 8.0 |
| BA9 Alzheimer's | 9.4 | Temp Pole Alzbeimer's2 | 8.0 |
| BA9 Alzheimer's2 | 10.4 | Temp Pole Parkinson's | 33.2 |
| BA9 Parkinson's | 42.6 | Temp Pole Parkinson's2 | 26.1 |
| BA9 Parkinson's2 | 25.9 | Temp Pole Huntington's | 22.2 |
| BA9 Huntington's | 11.6 | Temp Pole PSP | 6.0 |
| BA9 | 29.9 | Temp Pole PSP2 | 2.3 |
| Huntington's2 | | | |
| BA9 PSP | 11.0 | Temp Pole Depression2 | 15.1 |
| BA9 PSP2 | 9.5 | Cing Gyr Control | 34.2 |
| BA9 Depression | 16.4 | Cing Gyr Control2 | 13.2 |
| BA9 Depression2 | 12.8 | Cing Gyr Alzheimer's | 3.2 |
| BA17 Control | 68.3 | Cing Gyr Alzheimer's2 | 6.9 |
| BA17 Control2 | 28.3 | Cing Gyr Parkinson's | 17.9 |
| BA17 | 18.7 | Cing Gyr Parkinson's2 | 14.8 |
| Alzheimer's2 | | | |
| BA17 Parkinson's | 48.3 | Cing Gyr Huntington's | 6.6 |
| BA17 | 100.0 | Cing Gyr Huntington's2 | 9.7 |
| Parkinson's2 | | | |
| BA17 | 29.5 | Cing Gyr PSP | 2.2 |
| Huntington's | | | |
| BA17 | 32.3 | Cing Gry PSP2 | 6.1 |
| Huntington's2 | | | |
| BA17 Depression | 21.0 | Cing Gyr Depression | 5.2 |
| BA17 Depression2 | 53.2 | Cing Gyr Depression2 | 8.8 |

CNS_neurodegeneration_v1.0 Summary: Ag3217 Two experiments with the same probe and primer set show that the CG57362-01 gene exhibits decreased expression in the temporal cortex of brains suffering from Alzheimer's disease (p

Panel 1.3D Summary: Ag3217 Highest expression of the CG57362-01 gene is seen in a lung cancer cell line (CT=30.1). Significant expression is also seen in a cluster of lung cancer cell lines and samples derived from the brain. Thus, expression of this gene could be used to differentiate between brain-derived samples, the lung cancer cell lines and the other samples on this panel. Furthermore, therapeutic modulation of the expression or function of this gene may be effective in the treatment of lung cancer.

Among tissues with metabolic function, this gene is expressed in the adrenal and pituitary glands, and the pancreas. Thus, this gene product may be useful in the diagnosis and/or treatment of metabolic disease, including obesity and diabetes.

In addition, brain-preferential expression of thsi gene indicates that drugs that target this potential mediator of Alzheimer's disease, as seen in CNS_neurodegeneration_V1.0, are likely to have brain-specific action, a desireable feature of effective drugs.

Panel 2.2 Summary: Ag3217 Expression of the CG57362-01 gene is restricted to a sample derived from ovarian tissue (CT=34.9). Thus, expression of this gene could be used to differentiate between this sample and other samples on this panel.

Panel 4D Summary: Ag3217 Expression of the CG57362-01 gene is restricted to a sample derived from the colon (CT=34.8). Therefore, expression of this gene may be used to distinguish colon from the other tissues on this panel. Furthermore, expression of this gene is decreased in colon samples from patients with IBD colitis and Crohn's disease relative to normal colon. Therefore, therapeutic modulation of the activity of the GPCR encoded by this gene may be useful in the treatment of inflammatory bowel disease

Panel CNS_{—}1 Summary: Ag3217 Expression in this panel confirms the presence of the CG57362-01 gene in the brain. Please see Panels 1.3D and CNS_neurodegeneration_v1.0 for discussion of utility of this gene in the central nervous system.

D. NOV4a and NOV4c: Lymphocyte Antigen Precursor

Expression of gene CG56417-01 and variant CG56417-03 was assessed using the primer-probe set Ag2917, described in Table DA. Results of the RTQ-PCR runs are shown in Tables DB, DC, DD, and DE.

TABLE DA

Probe Name Ag2917

| | | | |
|---|---|---|---|
| | | | Start |
| Pri- | | | Posi- |
| mers | Sequences | Length | tion |

| | | | |
|---|---|---|---|
| For- | 5′-cagtctccaggccatgaag-3′ | 19 | 36 |
| ward | (SEQ ID NO:42) | | |
| Probe | TET- | 23 | 55 |
| | 5′-accttgtccctggtcctgctggt-3′- | | |
| | TAMRA (SEQ ID NO:43) | | |
| Re- | 5′-agaccctgagctctctccat-3′ | 20 | 91 |
| verse | (SEQ ID NO:44) | | |

TABLE DB
CNS_neurodegeneration_v1.0

| | Rel. Exp.(%) | | Rel. Exp.(%) |
|---|---|---|---|
| | Ag2917, | | Ag2917, |
| Tissue Name | Run 209735957 | Tissue Name | Run 209735957 |
| AD 1 Hippo | 1.8 | Control (Path) 3 | 0.0 |
| | | Temporal Ctx | |
| AD 2 Hippo | 12.7 | Control (Path) 4 | 6.6 |
| | | Temporal Ctx | |
| AD 3 Hippo | 0.0 | AD 1 Occipital Ctx | 6.6 |
| AD 4 Hippo | 0.0 | AD 2 Occipital Ctx | 0.0 |
| | | (Missing) | |
| AD 5 hippo | 51.8 | AD 3 Occipital Ctx | 5.3 |
| AD 6 Hippo | 3.7 | AD 4 Occipital Ctx | 3.6 |
| Control 2 Hippo | 0.0 | AD 5 Occipital Ctx | 36.3 |
| Control 4 Hippo | 6.9 | AD 6 Occipital Ctx | 29.9 |
| Control (Path) 3 Hippo | 5.4 | Control 1 Occipital Ctx | 0.0 |
| AD 1 Temporal Ctx | 1.1 | Control 2 Occipital Ctx | 12.7 |
| AD 2 Temporal Ctx | 7.3 | Control 3 Occipital Ctx | 4.5 |
| AD 3 Temporal Ctx | 0.0 | Control 4 Occipital Ctx | 0.0 |
| AD 4 Temporal Ctx | 6.7 | Control (Path) 1 | 15.5 |
| | | Occipital Ctx | |
| AD 5 Inf Temporal Ctx | 100.0 | Control (Path) 2 | 0.0 |
| | | Occipital Ctx | |
| AD 5 SupTemporal Ctx | 7.7 | Control (Path) 3 | 4.5 |
| | | Occipital Ctx | |
| AD 6 Inf Temporal Ctx | 9.4 | Control (Path) 4 | 16.6 |
| | | Occipital Ctx | |
| AD 6 Sup Temporal Ctx | 0.0 | Control 1 Parietal Ctx | 0.0 |
| Control 1 Temporal Ctx | 0.0 | Control 2 Parietal Ctx | 17.0 |
| Control 2 Temporal Ctx | 8.9 | Control 3 Parietal Ctx | 9.5 |
| Control 3 Temporal Ctx | 19.8 | Control (Path) 1 | 19.5 |
| | | Parietal Ctx | |
| Control 4 Temporal Ctx | 0.0 | Control (Path) 2 | 1.6 |
| | | Parietal Ctx | |
| Control (Path) 1 | 31.2 | Control (Path) 3 | 4.5 |
| Temporal Ctx | | Parietal Ctx | |
| Control (Path) 2 | 13.5 | Control (Path) 4 | 19.5 |
| Temporal Ctx | | Parietal Ctx | |

TABLE DC
Panel 1.3D

| | Rel. Exp.(%) | Rel. Exp.(%) | | Rel. Exp.(%) | Rel. Exp.(%) |
|---|---|---|---|---|---|
| | Ag2917, Run | Ag2917, Run | | Ag2917, Run | Ag2917, Run |
| Tissue Name | 161411861 | 165721698 | Tissue Name | 161411861 | 165721698 |
| Liver | 0.0 | 0.0 | Kidney (fetal) | 0.0 | 0.0 |
| adenocarcinoma | | | | | |
| Pancreas | 0.0 | 0.0 | Renal ca. 786-0 | 0.0 | 0.0 |
| Pancreatic ca. | 2.5 | 25.2 | Renal ca. A498 | 6.5 | 18.7 |
| CAPAN 2 | | | | | |
| Adrenal gland | 0.0 | 0.0 | Renal ca. RXF | 0.0 | 0.0 |
| | | | 393 | | |
| Thyroid | 0.0 | 0.0 | Renal ca. ACHN | 0.4 | 0.0 |
| Salivary gland | 0.0 | 0.0 | Renal ca. UO-31 | 0.0 | 0.0 |
| Pituitary gland | 0.0 | 0.0 | Renal ca. TK-10 | 0.0 | 0.0 |
| Brain (fetal) | 0.0 | 0.0 | Liver | 0.0 | 0.0 |
| Brain (whole) | 0.0 | 100.0 | Liver (fetal) | 0.0 | 0.0 |
| Brain (amygdala) | 5.6 | 10.9 | Liver ca. | 0.0 | 0.0 |
| | | | (hepatoblast) | | |
| | | | HepG2 | | |
| Brain (cerebellum) | 6.5 | 2.5 | Lung | 0.0 | 0.0 |
| Brain (hippocampus) | 0.6 | 41.2 | Lung (fetal) | 0.0 | 6.7 |
| Brain (substantia | 0.0 | 0.0 | Lung ca. (small | 0.0 | 0.0 |
| nigra) | | | cell) LX-1 | | |
| Brain (thalamus) | 6.6 | 0.0 | Lung ca. (small | 0.0 | 0.0 |
| | | | cell) NCI-H69 | | |
| Cerebral Cortex | 100.0 | 17.1 | Lung ca. (s. cell | 0.0 | 0.0 |
| | | | var.) SHP-77 | | |
| Spinal cord | 12.9 | 26.2 | Lung ca. (large | 0.0 | 0.0 |
| | | | cell)NCI-H460 | | |
| glio/astro U87-MG | 1.7 | 0.0 | Lung ca. (non- | 0.0 | 0.0 |
| | | | sm. cell) A549 | | |
| glio/astro U-118- | 0.0 | 0.0 | Lung ca. (non- | 0.0 | 0.0 |
| MG | | | s. cell) NCI-H23 | | |
| astrocytoma | 6.4 | 0.0 | Lung ca. (non- | 0.0 | 0.0 |
| SW1783 | | | s. cell) HOP-62 | | |
| neuro*; met SK-N- | 0.5 | 13.4 | Lung ca. (non- | 0.3 | 0.0 |
| AS | | | s. cl) NCI-H522 | | |
| astrocytoma SF-539 | 0.0 | 0.0 | Lung ca. | 0.0 | 0.0 |
| | | | (squam.) SW 900 | | |
| astrocytoma SNB-75 | 0.0 | 0.0 | Lung ca. | 0.0 | 0.0 |
| | | | (squam.) NCI- | | |
| | | | H596 | | |
| glioma SNB-19 | 0.0 | 0.0 | Mammary gland | 0.0 | 0.0 |
| glioma U251 | 0.0 | 0.0 | Breast ca.* | 0.0 | 0.0 |
| | | | (pl.ef) MCF-7 | | |
| glioma SF-295 | 0.0 | 0.0 | Breast ca.* | 0.0 | 0.0 |
| | | | (pl.ef) MDA- | | |
| | | | MB-231 | | |
| Heart (fetal) | 2.1 | 0.0 | Breast ca.* | 0.0 | 0.0 |
| | | | (pl.ef) T47D | | |
| Heart | 0.0 | 12.6 | Breast ca. BT- | 0.0 | 0.0 |
| | | | 549 | | |
| Skeletal muscle | 9.0 | 0.0 | Breast ca. MDA-N | 5.5 | 0.0 |
| (fetal) | | | | | |
| Skeletal muscle | 0.4 | 38.4 | Ovary | 9.8 | 0.0 |
| Bone marrow | 0.0 | 0.0 | Ovarian ca. | 0.0 | 0.0 |
| | | | OVCAR-3 | | |
| Thymus | 7.4 | 0.0 | Ovarian ca. | 0.0 | 0.0 |
| | | | OVCAR-4 | | |
| Spleen | 13.4 | 37.6 | Ovarian ca. | 0.4 | 10.3 |
| | | | OVCAR-5 | | |
| Lymph node | 0.0 | 0.0 | Ovarian ca. | 0.8 | 17.0 |
| | | | OVCAR-8 | | |
| Colorectal | 0.0 | 0.0 | Ovarian ca. | 6.3 | 15.0 |
| | | | IGROV-1 | | |
| Stomach | 0.0 | 0.0 | Ovarian ca.* | 0.0 | 0.0 |
| | | | (ascites) SK-OV-3 | | |
| Small intestine | 0.0 | 0.0 | Uterus | 0.0 | 0.0 |
| Colon ca. SW480 | 0.0 | 7.7 | Placenta | 0.0 | 0.0 |
| Colon ca.* | 0.0 | 0.0 | Prostate | 0.0 | 0.0 |
| SW620(SW480 met) | | | | | |
| Colon ca. HT29 | 0.0 | 0.0 | Prostate ca.* | 0.0 | 0.0 |
| | | | (bone met)PC-3 | | |
| Colon ca. HCT-116 | 0.0 | 0.0 | Testis | 18.0 | 0.0 |
| Colon ca. CaCo-2 | 7.8 | 0.0 | Melanoma | 0.0 | 0.0 |
| | | | Hs688(A).T | | |
| Colon ca. | 0.0 | 0.0 | Melanoma* | 1.2 | 0.0 |
| tissue(ODO3866) | | | (met) | | |
| | | | Hs688(B).T | | |
| Colon ca. HCC-2998 | 0.0 | 0.0 | Melanoma | 0.0 | 0.0 |
| | | | UACC-62 | | |
| Gastric ca.* (liver | 44.1 | 71.7 | Melanoma M14 | 0.0 | 0.0 |
| met) NCI-N87 | | | | | |
| Bladder | 2.0 | 0.0 | Melanoma LOX | 0.0 | 0.0 |
| | | | IMVI | | |
| Trachea | 10.9 | 4.8 | Melanoma* | 0.0 | 0.0 |
| | | | (met) SK-MEL-5 | | |
| Kidney | 0.0 | 0.0 | Adipose | 9.2 | 0.0 |

TABLE DD
Panel 2D

| | Rel. Exp.(%) Ag2917, | | Rel. Exp.(%) Ag2917, |
|---|---|---|---|
| Tissue Name | Run 161463558 | Tissue Name | Run 161463558 |
| Normal Colon | 3.2 | Kidney Margin 8120608 | 0.0 |
| CC Well to Mod Diff | 0.0 | Kidney Cancer 8120613 | 0.0 |
| (ODO3866) | | | |
| CC Margin (ODO3866) | 0.0 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 0.8 | Kidney Margin 9010321 | 0.9 |
| CC Mod Diff (ODO3920) | 0.0 | Normal Uterus | 0.0 |
| CC Margin (ODO3920) | 2.0 | Uterus Cancer 064011 | 1.7 |
| CC Gr.2 ascend colon | 0.2 | Normal Thyroid | 3.8 |
| (ODO3921) | | | |
| CC Margin (ODO3921) | 0.0 | Thyroid Cancer 064010 | 0.1 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 6.0 |
| Colon mets to lung (OD04451- | 0.0 | Normal Breast | 0.0 |
| 01) | | | |
| Lung Margin (OD04451-02) | 0.0 | Breast Cancer (OD04566) | 0.0 |
| Normal Prostate 6546-1 | 1.6 | Breast Cancer (OD04590- | 0.0 |
| | | 01) | |
| Prostate Cancer (OD04410) | 0.2 | Breast Cancer Mets | 0.0 |
| | | (OD04590-03) | |
| Prostate Margin (OD04410) | 0.8 | Breast Cancer Metastasis | 0.0 |
| | | (OD04655-05) | |
| Prostate Cancer (OD04720-01) | 0.5 | Breast Cancer 064006 | 0.9 |
| Prostate Margin (OD04720-02) | 0.8 | Breast Cancer 1024 | 1.4 |
| Normal Lung 061010 | 0.0 | Breast Cancer 9100266 | 0.1 |
| Lung Met to Muscle | 1.7 | Breast Margin 9100265 | 0.0 |
| (ODO4286) | | | |
| Muscle Margin (ODO4286) | 2.0 | Breast Cancer A209073 | 1.8 |
| Lung Malignant Cancer | 4.2 | Breast Margin A209073 | 1.2 |
| (OD03126) | | | |
| Lung Margin (OD03126) | 1.7 | Normal Liver | 0.0 |
| Lung Cancer (OD04404) | 0.0 | Liver Cancer 064003 | 0.0 |
| Lung Margin (OD04404) | 0.1 | Liver Cancer 1025 | 0.0 |
| Lung Cancer (OD04565) | 0.9 | Liver Cancer 1026 | 0.0 |
| Lung Margin (OD04565) | 0.0 | Liver Cancer 6004-T | 0.4 |
| Lung Cancer (OD04237-01) | 0.0 | Liver Tissue 6004-N | 1.4 |
| Lung Margin (OD04237-02) | 0.0 | Liver Cancer 6005-T | 0.0 |
| Ocular Mel Met to Liver | 0.0 | Liver Tissue 6005-N | 0.0 |
| (ODO4310) | | | |
| Liver Margin (ODO4310) | 0.0 | Normal Bladder | 0.0 |
| Melanoma Mets to Lung | 0.0 | Bladder Cancer 1023 | 0.0 |
| (OD04321) | | | |
| Lung Margin (OD04321) | 0.9 | Bladder Cancer A302173 | 0.0 |
| Normal Kidney | 0.5 | Bladder Cancer | 100.0 |
| | | (OD04718-01) | |
| Kidney Ca, Nuclear grade 2 | 0.0 | Bladder Normal Adjacent | 0.0 |
| (OD04338) | | (OD04718-03) | |
| Kidney Margin (OD04338) | 0.0 | Normal Ovary | 0.0 |
| Kidney Ca Nuclear grade ½ | 0.0 | Ovarian Cancer 064008 | 0.8 |
| (OD04339) | | | |
| Kidney Margin (OD04339) | 1.2 | Ovarian Cancer | 0.0 |
| | | (OD04768-07) | |
| Kidney Ca, Clear cell type | 0.0 | Ovary Margin (OD04768- | 1.6 |
| (OD04340) | | 08) | |
| Kidney Margin (OD04340) | 0.1 | Normal Stomach | 0.0 |
| Kidney Ca, Nuclear grade 3 | 0.0 | Gastric Cancer 9060358 | 0.0 |
| (OD04348) | | | |
| Kidney Margin (OD04348) | 0.3 | Stomach Margin 9060359 | 0.0 |
| Kidney Cancer (OD04622-01) | 0.0 | Gastric Cancer 9060395 | 1.8 |
| Kidney Margin (OD04622-03) | 0.4 | Stomach Margin 9060394 | 14.5 |
| Kidney Cancer (OD04450-01) | 0.0 | Gastric Cancer 9060397 | 0.0 |
| Kidney Margin (OD04450-03) | 0.0 | Stomach Margin 9060396 | 0.0 |
| Kidney Cancer 8120607 | 0.0 | Gastric Cancer 064005 | 31.6 |

TABLE DE
Panel 3D

| | Rel. Exp.(%) | | Rel. Exp.(%) |
|---|---|---|---|
| | Ag2917, Run | | Ag2917, Run |
| Tissue Name | 164629842 | Tissue Name | 164629842 |
| Daoy-Medulloblastoma | 0.0 | Ca Ski-Cervical epidermoid | 0.0 |
| | | carcinoma (metastasis) | |
| TE671-Medulloblastoma | 0.0 | ES-2-Ovarian clear cell carcinoma | 0.0 |
| D283 Med-Medulloblastoma | 0.0 | Ramos-Stimulated with | 0.0 |
| | | PMA/ionomycin 6h | |
| PFSK-1-Primitive | 0.0 | Ramos-Stimulated with | 0.0 |
| Neuroectodermal | | PMA/ionomycin 14h | |
| XF-498-CNS | 0.0 | MEG-01-Chronic myelogenous | 0.0 |
| | | leukemia (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.0 |
| T98G-Glioblastoma | 0.0 | U266-B-cell plasmacytoma | 0.0 |
| SK-N-SH-Neuroblastoma | 0.0 | CA46-Burkitt's lymphoma | 0.0 |
| (metastasis) | | | |
| SF-295-Glioblastoma | 0.0 | RL-non-Hodgkin's B-cell | 0.0 |
| | | lymphoma | |
| Cerebellum | 7.5 | JM1-pre-B-cell lymphoma | 0.0 |
| Cerebellum | 0.0 | Jurkat-T cell leukemia | 0.0 |
| NCI-H292-Mucoepidermoid | 0.0 | TF-1-Erythroleukemia | 0.0 |
| lung carcinoma | | | |
| DMS-114-Small cell lung | 0.0 | HUT 78-T-cell lymphoma | 0.0 |
| cancer | | | |
| DMS-79-Small cell lung | 10.3 | U937-Histiocytic lymphoma | 4.3 |
| cancer | | | |
| NCI-H146-Small cell lung | 5.7 | KU-812-Myelogenous leukemia | 0.0 |
| cancer | | | |
| NCI-H526-Small cell lung | 0.0 | 769-P-Clear cell renal carcinoma | 0.0 |
| cancer | | | |
| NCI-N417-Small cell lung | 0.0 | Caki-2-Clear cell renal carcinoma | 2.5 |
| cancer | | | |
| NCI-H82-Small cell lung | 0.0 | SW 839-Clear cell renal carcinoma | 0.0 |
| cancer | | | |
| NCI-H157-Squamous cell | 0.0 | G401-Wilms' tumor | 0.0 |
| lung cancer (metastasis) | | | |
| NCI-H1155-Large cell lung | 0.0 | Hs766T-Pancreatic carcinoma (LN | 42.9 |
| cancer | | metastasis) | |
| NCI-H1299-Large cell lung | 0.0 | CAPAN-1-Pancreatic | 0.0 |
| cancer | | adenocarcinoma (liver metastasis) | |
| NCI-H727-Lung carcinoid | 0.0 | SU86.86-Pancreatic carcinoma | 0.6 |
| | | (liver mtastasis) | |
| NCI-UMC-11-Lung | 0.0 | BxPC-3-Pancreatic | 18.7 |
| carcinoid | | adenocarcinoma | |
| LX-1-Small cell lung cancer | 0.0 | HPAC-Pancreatic adenocarcinoma | 92.0 |
| Colo-205-Colon cancer | 0.0 | MIA PaCa-2-Pancreatic carcinoma | 0.0 |
| KM12-Colon cancer | 6.3 | CFPAC-1-Pancreatic ductal | 3.3 |
| | | adenocarcinoma | |
| KM20L2-Colon cancer | 0.0 | PANC-1-Pancreatic epithelioid | 0.0 |
| | | ductal carcinoma | |
| NCI-H716-Colon cancer | 0.0 | T24-Bladder carcinma (transitional | 0.0 |
| | | cell) | |
| SW-48-Colon | 0.0 | 5637-Bladder carcinoma | 0.0 |
| adenocarcinoma | | | |
| SW1116-Colon | 0.0 | HT-1197-Bladder carcinoma | 13.6 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 0.0 |
| carcinoma | | | |
| KATO III-Gastric carcinoma | 100.0 | SK-LMS-1-Leiomyosarcoma | 0.0 |
| | | (vulva) | |
| NCI-SNU-16-Gastric | 0.0 | SJRH30-Rhabdomyosarcoma (met | 0.0 |
| carcinoma | | to bone marrow) | |
| NCI-SNU-1-Gastric | 0.0 | A431-Epidermoid carcinoma | 0.0 |
| carcinoma | | | |
| RF-1-Gastric | 0.0 | WM266-4-Melanoma | 0.0 |
| adenocarcinoma | | | |
| RF-48-Gastric | 0.0 | DU 145-Prostate carcinoma (brain | 0.0 |
| adenocarcinoma | | metastasis) | |
| MKN-45-Gastric carcinoma | 0.0 | MDA-MB-468-Breast | 12.2 |
| | | adenocarcinoma | |
| NCI-N87-Gastric carcinoma | 4.0 | SCC-4-Squamous cell carcinoma | 0.0 |
| | | of tongue | |
| OVCAR-5-Ovarian | 3.4 | SCC-9-Squamous cell carcinoma | 0.0 |
| carcinoma | | of tongue | |
| RL95-2-Uterine carcinoma | 3.0 | SCC-15-Squamous cell carcinoma | 0.0 |
| | | of tongue | |
| HelaS3-Cervical | 0.0 | CAL 27-Squamous cell carcinoma | 0.0 |
| adenocarcinoma | | of tongue | |

CNS_neurodegeneration_v1.0 Summary: Ag2917 This panel does not show differential expression of the CG56417-01 gene in Alzheimer's disease. However, this expression profile confirms the presence of this gene in the brain. Please see Panel 1.3D for discussion of utility of this gene in the central nervous system.

Panel 1.3D Summary: Ag2917 Two experiments with the same probe and primer set both show highest expression of the CG56417-01 gene, a putative lymphocyte antigen, in regions of the brain (CTs=32-34). Preferential expression in the cerebral cortex indicates a role for this gene product in CNS processes. Lymphocytes are involved in the inflammatory response to brain pathologies such as trauma. Therefore, targeting this gene product with drugs or directing toxins to cells that specifically express this gene product may have utility in countering the inflammation that is thought to be part of the pathologic etiology of numerous CNS pathologies, such as stroke, trauma, and neurodegenerative disorders such as Alzheimer's, Parkinson's and Huntington's diseases.

Significant expression is also seen in a gastric cancer cell line. Thus, expression of this gene could be used to differentiate samples from the brain and the cancer cell line from other samples on this panel and as a marker for brain tissue and gastric cancer (Holmin et al., Neurosurgery 1998 February; 42 (2):291-8).

Panel 2D Summary: Ag2917 Highest expression of the CG56417-01 gene is seen in a bladder cancer (CT=29.4). Significant expression is also seen in a gastric cancer and normal thyroid. Thus, expression of this gene could be used to differentiate between these samples and other samples on this panel and as a marker for the presence of these cancers. Furthermore, therapeutic modulation of the expression or function of this gene may be effective in the treatment of bladder, gastric, or thyroid cancer.

Panel 3D Summary: Ag2917 Expression of the CG56417-01 gene is limited to cell lines derived from gastric cancer, pancreatic cancer, and bladder cancer (CTs=32-35). This expression is consistent with expression in Panel 2D. Thus, expression of this gene could be used to differentiate between these samples and other samples on this panel.

Panel 4.1D Summary: Ag2917 Expression of the CG56417-01 gene is low/undetectable in all samples on this panel (CTs>35). (Data not shown.)

Panel 4D Summary: Ag2917 Results from one experiment with the CG56417-01 gene are not included. The amp plot indicates that there were experimental difficulties with this run.

E. NOV4b and NOV4d: Lymphocyte Antigen Like

Expression of gene CG56417-02 and variant CG56417-04 was assessed using the primer-probe set Ag4251, described in Table EA.

TABLE EA

Probe Name Ag4251

| | | | |
|---|---|---|---|
| Pri- | | | Start |
| mers | Sequences | Length | Position |

| | | | |
|---|---|---|---|
| For- | 5′-gtgttgctaggaagcatgttct-3′ | 22 | 87 |
| ward | (SEQ ID NO:45) | | |
| Probe | TET-5′- | 25 | 128 |
| | acacgtgcagtagctgcacctgctt-3′- | | |
| | TAMRA (SEQ ID NO:46) | | |
| Re- | 5′-atgacacatgcgacgtatgag-3′ | 21 | 154 |
| verse | (SEQ ID NO:47) | | |

CNS_neurodegeneration_v1.0 Summary: Ag4251 Expression of the CG56417-02 gene is low/undetectable in all samples on this panel (CTs>35). The amp plot indicates that there is a high probability of a probe failure.

General_screening_panel_v1.4 Summary: Ag4251 Expression of the CG56417-02 gene is low/undetectable in all samples on this panel (CTs>35). The amp plot indicates that there is a high probability of a probe failure.

Panel 4.1D Summary: Ag4251 Expression of the CG56417-02 gene is low/undetectable in all samples on this panel (CTs>35). The amp plot indicates that there is a high probability of a probe failure.

F. NOV5: TASK-4

Expression of gene CG57220-04 was assessed using the primer-probe sets Ag4293 and Ag937, described in Tables FA and FB. Results of the RTQ-PCR runs are shown in Tables FC, FD and FE.

TABLE FA

Probe Name Ag4293

| | | | |
|---|---|---|---|
| | | | Start |
| Pri- | | | Posi- |
| mers | Sequences | Length | tion |

| | | | |
|---|---|---|---|
| For- | 5′-gaggtacccactgtggtacaag-3′ | 22 | 733 |
| ward | (SEQ ID NO:48) | | |
| Probe | TET-5′- | 26 | 756 |
| | acatggtgtccctgtggatcctcttt-3′- | | |
| | TAMRA (SEQ ID NO:49) | | |
| Re- | 5′-gggagaggatgagtttgatgat-3′ | 22 | 803 |
| verse | (SEQ ID NO:50) | | |

TABLE FB

Probe Name Ag937

| | | | |
|---|---|---|---|
| | | | Start |
| Pri- | | | Posi- |
| mers | Sequences | Length | tion |

| | | | |
|---|---|---|---|
| For- | 5′-ggctccttcttcttttctgtgt-3′ | 22 | 341 |
| ward | (SEQ ID NO:51) | | |
| Probe | TET-5′- | | |
| | atcaccaccattggctatggcaacct-3′- | 26 | 368 |
| | TAMRA (SEQ ID NO:52) | | |
| Re- | 5′-ggcaaagaagatgcagaagag-3′ | 21 | 419 |
| verse | (SEQ ID NO:53) | | |

TABLE FC
CNS_neurodegeneration_v1.0

| | Rel. Exp.(%) Ag4293, Run | | Rel. Exp.(%) Ag4293, Run |
|---|---|---|---|
| Tissue Name | 224073647 | Tissue Name | 224073647 |
| AD 1 Hippo | 19.5 | Control (Path) 3 | 0.0 |
| | | Temporal Ctx | |
| AD 2 Hippo | 52.9 | Control (Path) 4 | 24.0 |
| | | Temporal Ctx | |
| AD 3 Hippo | 0.0 | AD 1 Occipital Ctx | 18.8 |
| AD 4 Hippo | 10.6 | AD 2 Occipital Ctx | 0.0 |
| | | (Missing) | |
| AD 5 Hippo | 26.4 | AD 3 Occipital Ctx | 0.0 |
| AD 6 Hippo | 82.4 | AD 4 Occipital Ctx | 11.4 |
| Control 2 Hippo | 50.7 | AD 5 Occipital Ctx | 33.4 |
| Control 4 Hippo | 49.0 | AD 6 Occipital Ctx | 23.3 |
| Control (Path) 3 Hippo | 11.7 | Control 1 Occipital Ctx | 8.5 |
| AD 1 Temporal Ctx | 20.9 | Control 2 Occipital Ctx | 52.5 |
| AD 2 Temporal Ctx | 47.6 | Control 3 Occipital Ctx | 0.0 |
| AD 3 Temporal Ctx | 0.0 | Control 4 Occipital Ctx | 0.0 |
| AD 4 Temporal Ctx | 3.2 | Control (Path) 1 | 27.9 |
| | | Occipital Ctx | |
| AD 5 Inf Temporal Ctx | 49.3 | Control (Path) 2 | 42.3 |
| | | Occipital Ctx | |
| AD 5 Sup Temporal | 27.5 | Control (Path) 3 | 17.9 |
| Ctx | | Occipital Ctx | |
| AD 6 Inf Temporal Ctx | 12.4 | Control (Path) 4 | 35.6 |
| | | Occipital Ctx | |
| AD 6 Sup Temporal | 13.6 | Control 1 Parietal Ctx | 10.0 |
| Ctx | | | |
| Control 1 Temporal Ctx | 12.7 | Control 2 Parietal Ctx | 0.0 |
| Control 2 Temporal Ctx | 3.1 | Control 3 Parietal Ctx | 0.0 |
| Control 3 Temporal Ctx | 0.0 | Control (Path) 1 | 24.7 |
| | | Parietal Ctx | |
| Control 3 Temporal Ctx | 0.0 | Control (Path) 2 | 53.2 |
| | | Parietal Ctx | |
| Control (Path) 1 | 100.0 | Control (Path) 3 | 10.8 |
| Temporal Ctx | | Parietal Ctx | |
| Control (Path) 2 | 52.5 | Control (Path) 4 | 56.6 |
| Temporal Ctx | | Parietal Ctx | |

TABLE FD
General_screening_panel_v1.4

| | Rel. Exp.(%) Ag4293, Run | | Rel. Exp.(%) Ag4293, Run |
|---|---|---|---|
| Tissue Name | 222182574 | Tissue Name | 222182574 |
| Adipose | 3.0 | Renal ca. TK-10 | 0.0 |
| Melanoma* Hs688(A).T | 0.0 | Bladder | 5.9 |
| Melanoma* Hs688(B).T | 0.0 | Gastric ca. (liver met.) | 0.0 |
| | | NCI-N87 | |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 3.3 | Colon ca. HT29 | 0.0 |
| Prostate ca.* (bone met) | 0.0 | Colon ca. HCT-116 | 0.0 |
| PC-3 | | | |
| Prostate Pool | 1.5 | Colon ca. CaCo-2 | 0.0 |
| Placenta | 7.5 | Colon cancer tissue | 2.1 |
| Uterus Pool | 2.5 | Colon ca. SW1116 | 0.0 |
| Ovarian ca. OVCAR-3 | 0.2 | Colon ca. Colo-205 | 0.0 |
| Ovarian ca. SK-OV-3 | 0.1 | Colon ca. SW-48 | 0.0 |
| Ovarian ca. OVCAR-4 | 0.2 | Colon Pool | 7.6 |
| Ovarian ca. OVCAR-5 | 0.5 | Small Intestine Pool | 3.1 |
| Ovarian ca. IGROV-1 | 0.0 | Stomach Pool | 4.2 |
| Ovarian ca. OVCAR-8 | 0.0 | Bone Marrow Pool | 1.2 |
| Ovary | 0.5 | Fetal Heart | 3.2 |
| Breast ca. MCF-7 | 0.2 | Heart Pool | 5.8 |
| Breast ca. MDA-MB- | 0.0 | Lymph Node Pool | 4.0 |
| 231 | | | |
| Breast ca. BT 549 | 0.0 | Fetal Skeletal Muscle | 4.6 |
| Breast ca. T47D | 0.6 | Skeletal Muscle Pool | 0.0 |
| Breast ca. MDA-N | 0.4 | Spleen Pool | 0.7 |
| Breast Pool | 3.1 | Thymus Pool | 7.4 |
| Trachea | 1.2 | CNS cancer (glio/astro) | 0.0 |
| | | U87-MG | |
| Lung | 2.0 | CNS cancer (glio/astro) U- | 0.0 |
| | | 118-MG | |
| Fetal Lung | 100.0 | CNS cancer (neuro; met) | 0.0 |
| | | SK-N-AS | |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 71.2 | CNS cancer (glio) SNB-19 | 0.2 |
| Lung ca. SHP-77 | 0.0 | CNS cancer (glio) SF-295 | 0.0 |
| Lung ca. A549 | 0.0 | Brain (Amygdala) Pool | 1.5 |
| Lung ca. NCI-H526 | 0.0 | Brain (cerebellum) | 1.7 |
| Lung ca. NCI-H23 | 0.2 | Brain (fetal) | 0.2 |
| Lung ca. NCI-H460 | 0.0 | Brain (Hippocampus) Pool | 1.6 |
| Lung ca. HOP-62 | 0.0 | Cerebral Cortex Pool | 1.1 |
| Lung ca. NCI-H522 | 0.0 | Brain (Substantia nigra) | 0.8 |
| | | Pool | |
| Liver | 1.0 | Brain (Thalamus) Pool | 0.5 |
| Fetal Liver | 1.3 | Brain (whole) | 1.4 |
| Liver ca. HepG2 | 0.0 | Spinal Cord Pool | 2.0 |
| Kidney Pool | 10.2 | Adrenal Gland | 0.6 |
| Fetal Kidney | 1.9 | Pituitary gland Pool | 1.2 |
| Renal ca. 786-0 | 0.0 | Salivary Gland | 0.4 |
| Renal ca. A498 | 0.0 | Thyroid (female) | 6.0 |
| Renal ca. ACHN | 0.0 | Pancreatic ca. CAPAN2 | 0.0 |
| Renal ca. UO-31 | 0.0 | Pancreas Pool | 3.1 |

TABLE FE
Panel 4.1D

| | Rel. Exp.(%) Ag4293, | | Rel. Exp.(%) Ag4293, |
|---|---|---|---|
| Tissue Name | Run 181981932 | Tissue Name | Run 181981932 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 2.8 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC none | 0.0 |
| Primary Tr1 act | 0.0 | Microvascular Dermal EC | 0.0 |
| | | TNF alpha + IL-1beta | |
| Primary Th1 rest | 0.0 | Bronchial epithelium TNF alpha + IL1beta | 0.0 |
| Primary Th2 rest | 1.8 | Small airway epithelium none | 0.0 |
| Primary Th1 rest | 0.0 | Small airway epithelium | 0.0 |
| | | TNF alpha + IL-1beta | |
| CD45RA CD4 lymphocyte | 0.0 | Coronery artery SMC rest | 0.0 |
| act | | | |
| CD45RO CD4 lymphocyte | 0.0 | Coronery artery SMC TNF alpha + IL-1beta | 0.0 |
| act | | | |
| CD8 lymphocyte act | 0.0 | Astrocytes rest | 0.0 |
| Secondary CD8 | 0.0 | Astrocytes TNF alpha + IL-1beta | 0.0 |
| lymphocyte rest | | | |
| Secondary CD8 | 0.0 | KU-812 (Basophil) rest | 1.0 |
| lymphocyte act | | | |
| CD4 lymphocyte none | 2.0 | KU-812 (Basophil) | 0.0 |
| | | PMA/ionomycin | |
| 2ry Th1/Th2/Tr1_anti- | 0.0 | CCD1106 (Keratinocytes) none | 0.0 |
| CD95 CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.9 |
| LAK cells IL-2 + IL-12 | 0.0 | NCI-H292 none | 6.7 |
| LAK cells IL-2 + IFN | 0.0 | NCI-H292 IL-4 | 20.2 |
| gamma | | | |
| LAK cells IL-2 + IL-18 | 0.0 | NCI-H292 IL-9 | 10.7 |
| LAK cells | 0.0 | NCI-H292 IL-13 | 29.1 |
| PMA/ionomycin | | | |
| NK Cells IL-2 rest | 4.9 | NCI-H292 IFN gamma | 12.9 |
| Two Way MLR 3 day | 0.8 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 2.1 | Lung fibroblast TNF alpha + IL- | 0.0 |
| | | 1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L | 0.0 | Dermal fibroblast CCD1070 | 0.7 |
| and IL-4 | | TNF alpha | |
| EOL-1 dbcAMP | 8.1 | Dermal fibroblast CCD1070 IL- | 1.0 |
| | | 1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 4.0 |
| Monocytes LPS | 0.0 | Colon | 4.8 |
| Macrophages rest | 0.0 | Lung | 100.0 |
| Macrophages LPS | 0.0 | Thymus | 24.0 |
| HUVEC none | 0.0 | Kidney | 77.9 |
| HUVEC starved | 0.0 | | |

CNS_neurodegeneration_v1.0 Summary: Ag4293 No change is detected in the expression of the CG57220 gene in the postmortem Alzheimer's diseased brain when compared to controls. However this panel confirms the expression of this gene in the CNS in an independent group of patients. Thus, therapeutic modulation of the expression or function of this gene product may be useful in the treatment of neurologic disease. A second experiment with the probe primer set Ag937 showed low/undetectable levels of expression in all the samples on this panel (CTs>35). (Data not shown.)

General_screening_panel_v1.4 Summary: Ag4293 Highest expression of the CG57220 gene is seen in the fetal lung (CT=29)and a lung cancer cell line. In addition, this gene appears to be expressed at much higher levels in the fetal tissue than in the adult (CT=34.7). Higher levels of expression are also seen in fetal skeletal muscle C(T=33) than in adult skeletal muscle. Thus, expression of this gene could be used to differentiate between the two lung derived samples and other samples on this panel and as a marker to detect the presence of lung cancer. In addition, expression of this gene could be used to differentiate between the two sources of lung and skeletal muscle tissue. Furthermore, therapeutic modulation of the expression or function of this gene may be effective in the treatment of lung cancer.

Among tissues with metabolic function, this gene is expressed at low but significant levels in adipose, thyroid, pancreas, and adult and fetal heart. This widespread expression among these tissues suggests that this gene product may be useful for the diagnosis and/or treatment of metabolic disease, including obesity and diabetes.

Panel 4.1D Summary: Ag4293 Highest expression of the CG57220 gene is seen in the lung (CT=31). Significant levels of expression are also seen in a cluster of treated and untreated samples derived from the muco-epidermoid cell line NCI-H292, often used as a model for airway epithelium. The prominent expression in lung derived samples, both in this panel and General_screening_panel_v1.4 suggests that this gene may be involved in normal lung homeostasis as well as pathological and inflammatory lung conditions. Therefore, therapeutic modulation of this gene product may resuce symptoms associated with chronic obstructive pulmonary disease, asthma, allergy and emphysema.

G. NOV7: Cytokeratin

Expression of gene CG57454-01 was assessed using the primer-probe set Ag3244, described in Table GA. Results of the RTQ-PCR runs are shown in Tables GB, GC, GD and GE.

TABLE GA

Probe Name Ag3244

| | | | |
|---|---|---|---|
| Pri- | | | Start |
| mers | Sequences | Length | Position |

| | | | |
|---|---|---|---|
| For- | 5′-gctatgcaggtggtctgagtt-3′ | 21 | 1267 |
| ward | (SEQ ID NO:54) | | |
| Probe | TET-5′- | 24 | 1302 |
| | atcaccaccattggctatggcaacct-3′ | | |
| | (SEQ ID NO:55) | | |
| Re- | 5′-agagccaaagctggagctta-3′ | 20 | 1330 |
| verse | (SEQ ID NO:56) | | |

TABLE GB
CNS_neurodegeneration_v1.0

| | Rel. Exp.(%) Ag3244, Run | | Rel. Exp.(%) Ag3244, Run |
|---|---|---|---|
| Tissue Name | 206533582 | Tissue Name | 206533582 |
| AD 1 Hippo | 18.7 | Control (Path) 3 | 0.0 |
| | | Temporal Ctx | |
| AD 2 Hippo | 23.0 | Control (Path) 4 | 19.6 |
| | | Temporal Ctx | |
| AD 3 Hippo | 2.3 | AD 1 Occipital Ctx | 9.6 |
| AD 4 Hippo | 9.9 | AD 2 Occipital Ctx | 0.0 |
| | | (Missing) | |
| AD 5 hippo | 71.7 | AD 3 Occipital Ctx | 6.0 |
| AD 6 Hippo | 42.0 | AD 4 Occipital Ctx | 31.6 |
| Control 2 Hippo | 29.1 | AD 5 Occipital Ctx | 4.2 |
| Control 4 Hippo | 22.8 | AD 6 Occipital Ctx | 34.4 |
| Control (Path) 3 Hippo | 0.4 | Control 1 Occipital Ctx | 2.7 |
| AD 1 Temporal Ctx | 69.3 | Control 2 Occipital Ctx | 30.6 |
| AD 2 Temporal Ctx | 23.0 | Control 3 Occipital Ctx | 33.0 |
| AD 3 Temporal Ctx | 4.4 | Control 4 Occipital Ctx | 12.9 |
| AD 4 Temporal Ctx | 33.4 | Control (Path) 1 | 88.3 |
| | | Occipital Ctx | |
| AD 5 Inf Temporal Ctx | 100.0 | Control (Path) 2 | 9.0 |
| | | Occipital Ctx | |
| AD 5 SupTemporal Ctx | 23.8 | Control (Path) 3 | 0.0 |
| | | Occipital Ctx | |
| AD 6 Inf Temporal Ctx | 57.0 | Control (Path) 4 | 11.7 |
| | | Occipital Ctx | |
| AD 6 Sup Temporal Ctx | 42.0 | Control 1 Parietal Ctx | 24.5 |
| Control 1 Temporal Ctx | 4.4 | Control 2 Parietal Ctx | 59.5 |
| Control 2 Temporal Ctx | 28.7 | Control 3 Parietal Ctx | 4.5 |
| Control 3 Temporal Ctx | 15.5 | Control (Path) 1 | 59.0 |
| | | Parietal Ctx | |
| Control 4 Temporal Ctx | 5.3 | Control (Path) 2 | 23.2 |
| | | Parietal Ctx | |
| Control (Path) 1 | 28.7 | Control (Path) 3 | 0.0 |
| Temporal Ctx | | Parietal Ctx | |
| Control (Path) 2 | 32.5 | Control (Path) 4 | 37.6 |
| Temporal Ctx | | Parietal Ctx | |

TABLE GC
Panel 1.3D

| | Rel. Exp.(%) Ag3244, Run | | Rel. Exp.(%) Ag3244, Run |
|---|---|---|---|
| Tissue Name | 165524419 | Tissue Name | 165524419 |
| Liver adenocarcinoma | 91.4 | Kidney (fetal) | 10.6 |
| Pancreas | 3.4 | Renal ca. 786-0 | 8.3 |
| Pancreatic ca. CAPAN 2 | 23.7 | Renal ca. A498 | 29.1 |
| Adrenal gland | 0.0 | Renal ca. RXF 393 | 1.7 |
| Thyroid | 13.4 | Renal ca. ACHN | 10.9 |
| Salivary gland | 4.1 | Renal ca. UO-31 | 42.3 |
| Pituitary gland | 57.0 | Renal ca. TK-10 | 9.4 |
| Brain (fetal) | 0.0 | Liver | 5.4 |
| Brain (whole) | 48.3 | Liver (fetal) | 12.5 |
| Brain (amygdala) | 82.9 | Liver ca. (hepatoblast) | 18.3 |
| | | HepG2 | |
| Brain (cerebellum) | 36.3 | Lung | 9.4 |
| Brain (hippocampus) | 88.3 | Lung (fetal) | 1.7 |
| Brain (substantia nigra) | 63.3 | Lung ca. (small cell) LX-1 | 52.5 |
| Brain (thalamus) | 41.8 | Lung ca. (small cell) | 1.7 |
| | | NCI-H69 | |
| Cerebral Cortex | 7.1 | Lung ca. (s. cell var.) | 19.3 |
| | | SHP-77 | |
| Spinal cord | 60.7 | Lung ca. (large cell)NCI- | 9.5 |
| | | H460 | |
| glio/astro U87-MG | 0.0 | Lung ca. (non-sm. cell) | 20.6 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 10.2 |
| | | HOP-62 | |
| neuro*; met SK-N-AS | 7.4 | Lung ca. (non-s. cl) NCI- | 0.0 |
| | | H522 | |
| astrocytoma SF-539 | 0.0 | Lung ca. (squam.) SW | 7.4 |
| | | 900 | |
| astrocytoma SNB-75 | 17.1 | Lung ca. (squam.) NCI- | 10.6 |
| | | H596 | |
| glioma SNB-19 | 2.4 | Mammary gland | 7.0 |
| glioma U251 | 2.6 | Breast ca.* (pl.ef) MCF-7 | 80.7 |
| glioma SF-295 | 0.0 | Breast ca.* (pl.ef) MDA- | 34.9 |
| | | MB-231 | |
| Heart (fetal) | 3.1 | Breast ca.* (pl.ef) T47D | 21.8 |
| Heart | 2.7 | Breast ca. BT-549 | 0.0 |
| Skeletal muscle (fetal) | 0.0 | Breast ca. MDA-N | 0.0 |
| Skeletal muscle | 0.0 | Ovary | 6.0 |
| Bone marrow | 0.0 | Ovarian ca. OVCAR-3 | 37.6 |
| Thymus | 0.0 | Ovarian ca. OVCAR-4 | 22.5 |
| Spleen | 0.0 | Ovarian ca. OVCAR-5 | 98.6 |
| Lymph node | 0.0 | Ovarian ca. OVCAR-8 | 5.2 |
| Colorectal | 9.3 | Ovarian ca. IGROV-1 | 6.0 |
| Stomach | 15.1 | Ovarian ca.* (ascites) | 5.2 |
| | | SK-OV-3 | |
| Small intestine | 39.8 | Uterus | 0.0 |
| Colon ca. SW480 | 70.7 | Placenta | 52.1 |
| Colon ca.* SW620(SW480 | 9.2 | Prostate | 19.1 |
| met) | | | |
| Colon ca. HT29 | 27.2 | prostate ca.* (bone | 0.0 |
| | | met)PC-3 | |
| Colon ca. HCT-116 | 23.5 | Testis | 20.4 |
| Colon ca. CaCo-2 | 22.7 | Melanoma Hs688(A).T | 0.0 |
| Colon ca. | 85.9 | Melanoma* (met) | 0.0 |
| tissue(ODO3866) | | Hs688(B).T | |
| Colon ca. HCC-2998 | 17.8 | Melanoma UACC-62 | 0.0 |
| Gastric ca.* (liver met) | 100.0 | Melanoma M14 | 0.0 |
| NCI-N87 | | | |
| Bladder | 9.5 | Melanoma LOX IMVI | 0.0 |
| Trachea | 19.9 | Melanoma* (met) SK- | 0.0 |
| | | MEL-5 | |
| Kidney | 8.1 | Adipose | 3.8 |

TABLE GD
Panel 2.2

| | Rel. Exp.(%) Ag3244, | | Rel. Exp.(%) Ag3244, |
|---|---|---|---|
| Tissue Name | Run 174441100 | Tissue Name | Run 174441100 |
| Normal Colon | 31.0 | Kidney Margin (OD04348) | 12.8 |
| Colon cancer (OD06064) | 59.0 | Kidney malignant cancer | 25.0 |
| | | (OD06204B) | |
| Colon Margin (OD06064) | 8.0 | Kidney normal adjacent | 6.5 |
| | | tissue (OD06204E) | |
| Colon cancer (OD06159) | 100.0 | Kidney Cancer (OD04450- | 39.5 |
| | | 01) | |
| Colon Margin (OD06159) | 43.2 | Kidney Margin (OD04450- | 2.2 |
| | | 03) | |
| Colon cancer (OD06297-04) | 16.0 | Kidney Cancer 8120613 | 6.3 |
| Colon Margin (OD06297-05) | 38.7 | Kidney Margin 8120614 | 11.3 |
| CC Gr.2 ascend colon | 23.5 | Kidney Cancer 9010320 | 2.0 |
| (ODO3921) | | | |
| CC Margin (ODO3921) | 20.7 | Kidney Margin 9010321 | 20.9 |
| Colon cancer metastasis | 12.9 | Kidney Cancer 8120607 | 17.1 |
| (OD06104) | | | |
| Lung Margin (OD06104) | 30.1 | Kidney Margin 8120608 | 4.4 |
| Colon mets to lung | 45.4 | Normal Uterus | 0.0 |
| (OD04451-01) | | | |
| Lung Margin (OD04451-02) | 3.3 | Uterine Cancer 064011 | 0.0 |
| Normal Prostate | 9.4 | Normal Thyroid | 8.1 |
| Prostate Cancer (OD04410) | 3.8 | Thyroid Cancer 064010 | 16.4 |
| Prostate Margin (OD04410) | 1.5 | Thyroid Cancer A302152 | 9.3 |
| Normal Ovary | 2.6 | Thyroid Margin A302153 | 4.5 |
| Ovarian cancer (OD06283- | 7.5 | Normal Breast | 58.6 |
| 03) | | | |
| Ovarian Margin (OD06283- | 1.3 | Breast Cancer (OD04566) | 16.0 |
| 07) | | | |
| Ovarian Cancer 064008 | 3.0 | Breast Cancer 1024 | 17.4 |
| Ovarian cancer (OD06145) | 8.0 | Breast Cancer (OD04590- | 59.0 |
| | | 01) | |
| Ovarian Margin (OD06145) | 3.2 | Breast Cancer Mets | 24.7 |
| | | (OD04590-03) | |
| Ovarian cancer (OD06455- | 10.4 | Breast Cancer Metastasis | 21.6 |
| 03) | | (OD04655-05) | |
| Ovarian Margin (OD06455- | 0.0 | Breast Cancer 064006 | 12.8 |
| 07) | | | |
| Normal Lung | 1.4 | Breast Cancer 9100266 | 10.0 |
| Invasive poor diff, lung | 14.2 | Breast Margin 9100265 | 1.4 |
| adeno (ODO4945-01 | | | |
| Lung Margin (ODO4945-03) | 2.5 | Breast Cancer A209073 | 7.6 |
| Lung Malignant Cancer | 25.3 | Breast Margin A2090734 | 24.7 |
| (OD03126) | | | |
| Lung Margin (OD03126) | 3.7 | Breast cancer (OD06083) | 46.0 |
| Lung Cancer (OD05014A) | 6.7 | Breast cancer node | 22.7 |
| | | metastasis (OD06083) | |
| Lung Margin (OD05014B) | 1.3 | Normal Liver | 5.5 |
| Lung cancer (OD06081) | 1.0 | Liver Cancer 1026 | 22.7 |
| Lung Margin (OD06081) | 1.1 | Liver Cancer 1025 | 68.3 |
| Lung Cancer (OD04237-01) | 18.6 | Liver Cancer 6004-T | 19.8 |
| Lung Margin (OD04237-02) | 16.3 | Liver Tissue 6004-N | 11.6 |
| Ocular Melanoma Metastasis | 0.0 | Liver Cancer 6005-T | 62.4 |
| Ocular Melanoma Margin | 10.0 | Liver Tissue 6005-N | 31.4 |
| (Liver) | | | |
| Melanoma Metastasis | 11.3 | Liver Cancer 064003 | 7.4 |
| Melanoma Margin (Lung) | 4.1 | Normal Bladder | 31.2 |
| Normal Kidney | 7.3 | Bladder Cancer 1023 | 20.2 |
| Kidney Ca, Nuclear grade 2 | 12.0 | Bladder Cancer A302173 | 20.6 |
| (OD04338) | | | |
| Kidney Margin (OD04338) | 16.2 | Normal Stomach | 51.1 |
| Kidney Ca Nuclear grade ½ | 22.5 | Gastric Cancer 9060397 | 49.0 |
| (OD04339) | | | |
| Kidney Margin (OD04339) | 3.6 | Stomach Margin 9060396 | 50.3 |
| Kidney Ca, Clear cell type | 11.7 | Gastric Cancer 9060395 | 52.1 |
| (OD04340) | | | |
| Kidney Margin (OD04340) | 3.7 | Stomach Margin 9060394 | 39.5 |
| Kidney Ca, Nuclear grade 3 | 1.1 | Gastric Cancer 064005 | 46.3 |
| (OD04348) | | | |

TABLE GE
Panel 4D

| | Rel. Exp.(%) Ag3244, | | Rel. Exp.(%) Ag3244, |
|---|---|---|---|
| Tissue Name | Run 164390753 | Tissue Name | Run 164390753 |
| Secondary Th1 act | 0.0 | HUVEC IL-1beta | 0.0 |
| Secondary Th2 act | 0.0 | HUVEC IFN gamma | 3.9 |
| Secondary Tr1 act | 0.0 | HUVEC TNF alpha + IFN | 3.1 |
| | | gamma | |
| Secondary Th1 rest | 0.0 | HUVEC TNF alpha + IL4 | 0.0 |
| Secondary Th2 rest | 0.0 | HUVEC IL-11 | 5.1 |
| Secondary Tr1 rest | 0.0 | Lung Microvascular EC none | 27.5 |
| Primary Th1 act | 0.0 | Lung Microvascular EC | 39.5 |
| | | TNF alpha + IL-1beta | |
| Primary Th2 act | 0.0 | Microvascular Dermal EC none | 0.0 |
| Primary Tr1 act | 0.0 | Microsvasular Dermal EC | 6.3 |
| | | TNF alpha + IL-1beta | |
| Primary Th1 rest | 0.0 | Bronchial epithelium TNF alpha + IL1beta | 18.3 |
| Primary Th2 rest | 0.0 | Small airway epithelium none | 7.6 |
| Primary Tr1 rest | 0.0 | Small airway epithelium | 23.7 |
| | | TNF alpha + IL-1beta | |
| CD45RA CD4 lymphocyte | 2.8 | Coronery artery SMC rest | 0.0 |
| act | | | |
| CD45RO CD4 lymphocyte | 0.0 | Coronery artery SMC TNF alpha + IL-1beta | 0.0 |
| act | | | |
| CD8 lymphocyte act | 3.1 | Astrocytes rest | 0.6 |
| Secondary CD8 | 0.0 | Astrocytes TNF alpha + IL-1beta | 1.3 |
| lymphocyte rest | | | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CD1106 (Keratinocytes) none | 16.2 |
| CD95 CH11 | | | |
| LAK cells rest | 0.0 | CD1106 (Keratinocytes) | 25.0 |
| | | TNF alpha + IL-1beta | |
| LAK cells IL-2 | 0.7 | Liver cirrhosis | 40.1 |
| LAK cells IL-2 + IL-12 | 0.0 | Lupus kidney | 2.5 |
| LAK cells IL-2 + IFN | 0.0 | NCI-H292 none | 38.4 |
| gamma | | | |
| LAK cells IL-2 + IL-18 | 0.0 | NCI-H292 IL-4 | 100.0 |
| LAK cells | 0.0 | NCI-H292 IL-9 | 58.2 |
| PMA/ionomycin | | | |
| NK Cells IL-2 rest | 3.3 | NCI-H292 IL-13 | 59.0 |
| Two Way MLR 3 day | 0.0 | NCI-H292 IFN gamma | 87.7 |
| Two Way MLR 5 day | 0.0 | HPAEC none | 3.7 |
| Two Way MLR 7 day | 0.0 | HPAEC TNF alpha + IL-1beta | 0.0 |
| PBMC rest | 0.0 | Lung fibroblast none | 2.4 |
| PBMC PWM | 0.0 | Lung fibroblast TNF alpha + IL- | 0.0 |
| | | 1beta | |
| PBMC PHA-L | 0.0 | Lung fibroblast IL-4 | 27.7 |
| Ramos (B cell) none | 0.0 | Lung fibroblast IL-9 | 13.8 |
| Ramos (B cell) ionomycin | 0.0 | Lung fibroblast IL-13 | 11.1 |
| B lymphocytes PWM | 0.0 | Lung fibroblast IFN gamma | 15.4 |
| B lymphocytes CD40L | 0.0 | Dermal fibroblast CCD1070 rest | 1.0 |
| and IL-4 | | | |
| EOL-1 dbcAMP | 0.0 | Dermal fibroblast CCD1070 | 7.8 |
| | | TNF alpha | |
| EOL-1 dbcAMP | 0.0 | Dermal fibroblast CCD1070 IL- | 0.0 |
| PMA/ionomycin | | 1beta | |
| Dendritic cells none | 3.2 | Dermal fibroblast IFN gamma | 80.1 |
| Dendritic cells LPS | 0.0 | Dermal fibroblast IL-4 | 98.6 |
| Dendritic cells anti-CD40 | 0.8 | IBD Colitis 2 | 3.5 |
| Monocytes rest | 0.0 | IBD Crohn's | 3.8 |
| Monocytes LPS | 0.0 | Colon | 51.4 |
| Macrophages rest | 0.0 | Lung | 17.9 |
| Macrophages LPS | 0.0 | Thymus | 9.3 |
| HUVEC none | 10.0 | Kidney | 1.5 |
| HUVEC starved | 4.7 | | |

CNS_neurodegeneration_v1.0 Summary: Ag3244 No change is detected in the expression of the CG57454-01 gene in the postmortem Alzheimer's diseased brain when compared to controls. See panel 1.3 for discussion of utility.

Panel 1.3D Summary: Ag3244 The expression of the CG57454-01 gene appears to be highest in a sample derived from a gastric cancer cell line (NCI-N87)(CT=33.9). In addition, there appears to be substantial expression in samples derived from ovarian cancer cell lines and colon cancer cell lines. Thus, the expression of this gene could be used to distinguish NCI-N87 cells from other samples in the panel. Moreover, therapeutic modulation of this gene, through the use of small molecule drugs, protein therapeutics or antibodies could be of benefit in the treatment of gastric cancer, colon cancer or ovarian cancer.

Panel 2.2 Summary: Ag3244 The expression of the CG57454-01 gene appears to be highest in a sample derived from a colon cancer sample (CT=33.1). In addition there appears to be substantial expression in other samples derived from colon cancers. Thus, the expression of this gene could be used to distinguish colon cancer from other samples in the panel. Moreover, therapeutic modulation of this gene, through the use of small molecule drugs, protein therapeutics or antibodies could be of benefit in the treatment of colon cancer.

Panel 4D Summary: Ag3244 The CG57454-01 transcript is expressed in dermal fibroblasts, lung fibroblasts, NCI-H292 cells and lung microvascular cells. The transcript is also expressed in normal colon but not in colitis samples. Thus, the transcript or the protein it encodes could be used to differentiate these cells and tissues from other samples on this panel that do not express this gene. Therapeutically, the protein encoded by this transcript could be used to design treatments for colitis, psoriasis, arthritis, asthma and emphysema.

H. NOV8a: Protocadherin

Expression of gene CG57446-01 was assessed using the primer-probe set Ag3241, described in Table HA. Results of the RTQ-PCR runs are shown in Table HB.

TABLE HA

Probe Name Ag3241

| | | | |
|---|---|---|---|
| | | | Start |
| Pri- | | | Posi- |
| mers | Sequences | Length | tion |

| | | | |
|---|---|---|---|
| For- | 5′-cttgtgctagtgatcgacatca-3′ | 22 | 1251 |
| ward (SEQ ID NO:57) | | | |
| Probe | TET-5′- | 27 | 1285 |
| | ccccttattctcaatcctcctactacg-3′- | | |
| | TAMRA (SEQ ID NO:58) | | |
| Re- | 5′-ccgttgttttcgtttacgtaaa-3′ | 22 | 1312 |
| verse | (SEQ ID NO:59) | | |

TABLE HB
Panel 1.3D

| | Rel. Exp.(%) Ag3241, Run | | Rel. Exp.(%) Ag3241, Run |
|---|---|---|---|
| Tissue Name | 165524414 | Tissue Name | 165524414 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) | 0.0 |
| | | HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) | 0.0 |
| | | NCI-H69 | |
| Cerebral Cortex | 0.0 | Lung ca. (s. cell var.) | 0.0 |
| | | SHP-77 | |
| Spinal cord | 0.0 | Lung ca. (large cell)NCI- | 0.0 |
| | | H460 | |
| glio/astro U87-MG | 0.0 | Lung ca. (non-sm. cell) | 0.0 |
| | | A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 0.0 |
| | | HOP-62 | |
| neuro*; met SK-N-AS | 0.0 | Lung ca. (non-s. cl) NCI- | 0.0 |
| | | H522 | |
| astrocytoma SF-539 | 0.0 | Lung ca. (squam.) SW | 0.0 |
| | | 900 | |
| astrocytoma SNB-75 | 0.0 | Lung ca. (squam.) NCI- | 0.0 |
| | | H596 | |
| glioma SNB-19 | 0.0 | Mammary gland | 0.0 |
| glioma U251 | 0.0 | Breast ca.* (pl.ef) MCF-7 | 0.0 |
| glioma SF-295 | 0.0 | Breast ca.* (pl.ef) MDA- | 0.0 |
| | | MB-231 | |
| Heart (fetal) | 0.0 | Breast ca.* (pl.ef) T47D | 0.0 |
| Heart | 0.0 | Breast ca. BT-549 | 0.0 |
| Skeletal muscle (fetal) | 100.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* SW620(SW480 | 0.0 | Prostate | 0.0 |
| met) | | | |
| Colon ca. HT29 | 0.0 | Prostate ca.* (bone | 0.0 |
| | | met)PC-3 | |
| Colon ca. HCT-116 | 0.0 | Testis | 0.0 |
| Colon ca. CaCo-2 | 0.0 | Melanoma Hs688(A).T | 0.0 |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

Panel 1.3D Summary: Ag3241 Expression of the CG57446-01 gene is restricted to fetal skeletal muscle (CT=33.8). Thus, expression of this gene could be used to differentiate between fetal and adult skeletal muscle (CT=40). This gene encodes a protein that is homologous to cadherin, a cell adhesion molecule that has been implicated in muscle differentiation. Thus, the relative overexpression of this gene in fetal skeletal muscle suggests that the protein product may enhance muscular growth or development in the fetus and may also act in a regenerative capacity in the adult. Therefore, therapeutic modulation of the protein encoded by this gene could be useful in treatment of muscle related diseases. More specifically, treatment of weak or dystrophic muscle with the protein encoded by this gene could restore muscle mass or function (Goichberg et al., J Cell Sci 2001 April; 114 (Pt 7): 1309-19).

Panel 2.2 Summary: Ag3241 Expression of the CG57446-01 gene is low/undetectable in all samples on this panel (CTs>35). The amp plot indicates that there is a possibility of a probe failure.

Panel 4D Summary: Ag3241 Expression of the CG57446-01 gene is low/undetectable in all samples on this panel (CTs>35). The amp plot indicates that there is a possibility of a probe failure.

Panel CNS_{—}1 Summary: Ag3241 Expression of the CG57446-01 gene is low/undetectable in all samples on this panel (CTs>35). The amp plot indicates that there is a possibility of a probe failure.

I. NOV8b: Protocadherin

Expression of gene CG57444-01 was assessed using the primer-probe set Ag3240, described in Table IA. Results of the RTQ-PCR runs are shown in Tables IB, IC, ID, IE and IF.

TABLE IA

Probe Name Ag3240

| | | | |
|---|---|---|---|
| Pri- | | | Start |
| mers | Sequences | Length | Position |

| | | | |
|---|---|---|---|
| For- | 5′-ggagactgctttgagtcagttc-3′ | 22 | 2184 |
| ward | (SEQ ID NO:60) | | |
| Probe | TET-5′- | 23 | 2206 |
| | tctgctccaagtccggacctgtg-3′- | | |
| | TAMRA (SEQ ID NO: 61) | | |
| Re- | 5′-aattataggcatagggcaacgt-3′ | 22 | 2253 |
| verse | (SEQ ID NO:62) | | |

TABLE IB
CNS_neurodegeneration_v1.0

| | Rel. Exp.(%) Ag3240, Run | | Rel. Exp.(%) Ag3240, Run |
|---|---|---|---|
| Tissue Name | 210037849 | Tissue Name | 210037849 |
| AD 1 Hippo | 17.7 | Control (Path) 3 | 6.9 |
| | | Temporal Ctx | |
| AD 2 Hippo | 37.9 | Control (Path) 4 | 28.1 |
| | | Temporal Ctx | |
| AD 3 Hippo | 6.8 | AD 1 Occipital Ctx | 13.7 |
| AD 4 Hippo | 8.3 | AD 2 Occipital Ctx | 0.0 |
| | | (Missing) | |
| AD 5 Hippo | 55.1 | AD 3 Occipital Ctx | 8.7 |
| AD 6 Hippo | 100.0 | AD 4 Occipital Ctx | 21.8 |
| Control 2 Hippo | 23.5 | AD 5 Occipital Ctx | 8.4 |
| Control 4 Hippo | 23.5 | AD 6 Occipital Ctx | 17.6 |
| Control (Path) 3 Hippo | 8.4 | Control 1 Occipital Ctx | 4.3 |
| AD 1 Temporal Ctx | 18.3 | Control 2 Occipital Ctx | 24.0 |
| AD 2 Temporal Ctx | 32.5 | Control 3 Occipital Ctx | 14.2 |
| AD 3 Temporal Ctx | 6.3 | Control 4 Occipital Ctx | 13.0 |
| AD 4 Temporal Ctx | 24.7 | Control (Path) 1 | 51.4 |
| | | Occipital Ctx | |
| AD 5 Inf Temporal Ctx | 59.0 | Control (Path) 2 | 8.7 |
| | | Occipital Ctx | |
| AD 5 Sup Temporal | 53.6 | Control (Path) 3 | 4.9 |
| Ctx | | Occipital Ctx | |
| AD 6 Inf Temporal Ctx | 43.8 | Control (Path) 4 | 15.4 |
| | | Occipital Ctx | |
| AD 6 Sup Temporal | 56.6 | Control 1 Parietal Ctx | 11.6 |
| Ctx | | | |
| Control 1 Temporal Ctx | 9.6 | Control 2 Parietal Ctx | 37.1 |
| Control 2 Temporal Ctx | 14.6 | Control 3 Parietal Ctx | 15.5 |
| Control 3 Temporal Ctx | 8.9 | Control (Path) 1 | 36.3 |
| | | Parietal Ctx | |
| Control 3 Temporal Ctx | 9.0 | Control (Path) 2 | 22.7 |
| | | Parietal Ctx | |
| Control (Path) 1 | 34.9 | Control (Path) 3 | 5.8 |
| Temporal Ctx | | Parietal Ctx | |
| Control (Path) 2 | 19.3 | Control (Path) 4 | 37.1 |
| Temporal Ctx | | Parietal Ctx | |

TABLE IC
Panel 1.3D

| | Rel. Exp.(%) Ag3240, Run | | Rel. Exp.(%) Ag3240, Run |
|---|---|---|---|
| Tissue Name | 165524271 | Tissue Name | 165524271 |
| Liver adenocarcinoma | 2.5 | Kidney (fetal) | 6.0 |
| Pancreas | 1.7 | Renal ca. 786-0 | 3.2 |
| Pancreatic ca. CAPAN 2 | 3.5 | Renal ca. A498 | 3.7 |
| Adrenal gland | 6.6 | Renal ca. RXF 393 | 6.7 |
| Thyroid | 3.9 | Renal ca. ACHN | 2.0 |
| Salivary gland | 3.7 | Renal ca. UO-31 | 0.4 |
| Pituitary gland | 14.0 | Renal ca. TK-10 | 0.2 |
| Brain (fetal) | 23.3 | Liver | 1.9 |
| Brain (whole) | 19.3 | Liver (fetal) | 2.8 |
| Brain (amygdala) | 21.9 | Liver ca. (hepatoblast) | 0.4 |
| | | HepG2 | |
| Brain (cerebellum) | 48.6 | Lung | 5.9 |
| Brain (hippocampus) | 23.3 | Lung (fetal) | 2.3 |
| Brain (substantia nigra) | 9.5 | Lung ca. (small cell) LX-1 | 1.0 |
| Brain (thalamus) | 17.8 | Lung ca. (small cell) | 0.4 |
| | | NCI-H69 | |
| Cerebral Cortex | 10.2 | Lung ca. (s. cell var.) | 0.6 |
| | | SHP-77 | |
| Spinal cord | 29.7 | Lung ca. (large cell)NCI- | 3.5 |
| | | H460 | |
| glio/astro U87-MG | 1.0 | Lung ca. (non-sm. cell) | 0.1 |
| | | A549 | |
| glio/astro U-118-MG | 100.0 | Lung ca. (non-s. cell) | 11.5 |
| | | NCI-H23 | |
| astrocytoma SW1783 | 4.2 | Lung ca. (non-s. cell) | 5.2 |
| | | HOP-62 | |
| neuro*; met SK-N-AS | 34.6 | Lung ca. (non-s. cl) NCI- | 2.4 |
| | | H522 | |
| astrocytoma SF-539 | 3.9 | Lung ca. (squam.) SW | 5.8 |
| | | 900 | |
| astrocytoma SNB-75 | 6.6 | Lung ca. (squam.) NCI- | 0.9 |
| | | H596 | |
| glioma SNB-19 | 8.1 | Mammary gland | 14.7 |
| glioma U251 | 9.4 | Breast ca.* (pl.ef) MCF-7 | 4.8 |
| glioma SF-295 | 3.2 | Breast ca.* (pl.ef) MDA- | 4.9 |
| | | MB-231 | |
| Heart (fetal) | 3.6 | Breast ca.* (pl.ef) T47D | 3.9 |
| Heart | 8.7 | Breast ca. BT-549 | 5.8 |
| Skeletal muscle (fetal) | 11.0 | Breast ca. MDA-N | 2.5 |
| Skeletal muscle | 8.1 | Ovary | 31.2 |
| Bone marrow | 1.4 | Ovarian ca. OVCAR-3 | 1.5 |
| Thymus | 1.5 | Ovarian ca. OVCAR-4 | 0.5 |
| Spleen | 5.8 | Ovarian ca. OVCAR-5 | 11.2 |
| Lymph node | 7.5 | Ovarian ca. OVCAR-8 | 0.5 |
| Colorectal | 6.2 | Ovarian ca. IGROV-1 | 5.7 |
| Stomach | 3.3 | Ovarian ca.* (ascites) | 6.9 |
| | | SK-OV-3 | |
| Small intestine | 12.4 | Uterus | 15.8 |
| Colon ca. SW480 | 0.1 | Placenta | 4.3 |
| Colon ca.* SW620(SW480 | 0.2 | Prostate | 5.3 |
| met) | | | |
| Colon ca. HT29 | 0.2 | Prostate ca.* (bone | 4.1 |
| | | met)PC-3 | |
| Colon ca. HCT-116 | 1.0 | Testis | 4.2 |
| Colon ca. CaCo-2 | 0.8 | Melanoma Hs688(A).T | 6.3 |
| Colon ca. | 3.2 | Melanoma* (met) | 8.0 |
| tissue(ODO3866) | | Hs688(B).T | |
| Colon ca. HCC-2998 | 5.1 | Melanoma UACC-62 | 2.8 |
| Gastric ca.* (liver met) | 8.9 | Melanoma M14 | 5.1 |
| NCI-N87 | | | |
| Bladder | 13.3 | Melanoma LOX IMVI | 0.4 |
| Trachea | 5.5 | Melanoma* (met) SK- | 3.0 |
| | | MEL-5 | |
| Kidney | 6.7 | Adipose | 3.8 |

TABLE ID
Panel 2.2

| | Rel. Exp.(%) Ag3240, | | Rel. Exp.(%) Ag3240, |
|---|---|---|---|
| Tissue Name | Run 174443262 | Tissue Name | Run 174443262 |
| Normal Colon | 13.7 | Kidney Margin (OD04348) | 40.9 |
| Colon cancer (OD06064) | 12.9 | Kidney malignant cancer | 1.2 |
| | | (OD06204B) | |
| Colon Margin (OD06064) | 20.3 | Kidney normal adjacent | 11.7 |
| | | tissue (OD06204E) | |
| Colon cancer (OD06159) | 0.4 | Kidney Cancer (OD04450- | 9.0 |
| | | 01) | |
| Colon Margin (OD06159) | 18.7 | Kidney Margin (OD04450- | 9.5 |
| | | 03) | |
| Colon cancer (OD06297-04) | 5.6 | Kidney Cancer 8120613 | 1.4 |
| Colon Margin (OD06297-05) | 34.2 | Kidney Margin 8120614 | 14.1 |
| CC Gr.2 ascend colon | 0.9 | Kidney Cancer 9010320 | 3.4 |
| (ODO3921) | | | |
| CC Margin (ODO3921) | 4.6 | Kidney Margin 9010321 | 5.5 |
| Colon cancer metastasis | 0.8 | Kidney Cancer 8120607 | 7.0 |
| (OD06104) | | | |
| Lung Margin (OD06104) | 2.5 | Kidney Margin 8120608 | 9.5 |
| Colon mets to lung | 1.8 | Normal Uterus | 52.9 |
| (OD04451-01) | | | |
| Lung Margin (OD04451-02) | 10.9 | Uterine Cancer 064011 | 17.9 |
| Normal Prostate | 15.7 | Normal Thyroid | 2.7 |
| Prostate Cancer (OD04410) | 17.3 | Thyroid Cancer 064010 | 13.7 |
| Prostate Margin (OD04410) | 4.9 | Thyroid Cancer A302152 | 17.0 |
| Normal Ovary | 48.3 | Thyroid Margin A302153 | 8.4 |
| Ovarian cancer (OD06283- | 1.9 | Normal Breast | 42.6 |
| 03) | | | |
| Ovarian Margin (OD06283- | 14.1 | Breast Cancer (OD04566) | 1.7 |
| 07) | | | |
| Ovarian Cancer 064008 | 10.1 | Breast Cancer 1024 | 40.3 |
| Ovarian cancer (OD06145) | 41.2 | Breast Cancer (OD04590- | 8.4 |
| | | 01) | |
| Ovarian Margin (OD06145) | 37.1 | Breast Cancer Mets | 9.4 |
| | | (OD04590-03) | |
| Ovarian cancer (OD06455- | 2.6 | Breast Cancer Metastasis | 8.9 |
| 03) | | (OD04655-05) | |
| Ovarian Margin (OD06455- | 41.8 | Breast Cancer 064006 | 4.3 |
| 07) | | | |
| Normal Lung | 23.8 | Breast Cancer 9100266 | 16.4 |
| Invasive poor diff. lung | 1.8 | Breast Margin 9100265 | 18.4 |
| adeno (ODO4945-01 | | | |
| Lung Margin (ODO4945-03) | 21.3 | Breast Cancer A209073 | 4.1 |
| Lung Malignant Cancer | 10.4 | Breast Margin A2090734 | 16.4 |
| (OD03126) | | | |
| Lung Margin (OD03126) | 13.7 | Breast cancer (OD06083) | 34.2 |
| Lung Cancer (OD05014A) | 3.7 | Breast cancer node | 26.6 |
| | | metastasis (OD06083) | |
| Lung Margin (OD05014B) | 33.0 | Normal Liver | 9.7 |
| Lung cancer (OD06081) | 0.0 | Liver Cancer 1026 | 1.6 |
| Lung Margin (OD06081) | 15.3 | Liver Cancer 1025 | 9.2 |
| Lung Cancer (OD04237-01) | 1.1 | Liver Cancer 6004-T | 7.5 |
| Lung Margin (OD04237-02) | 31.6 | Liver Tissue 6004-N | 1.6 |
| Ocular Melanoma Metastasis | 0.6 | Liver Cancer 6005-T | 5.8 |
| Ocular Melanoma Margin | 4.5 | Liver Tissue 6005-N | 17.1 |
| (Liver) | | | |
| Melanoma Metastasis | 0.5 | Liver Cancer 064003 | 7.4 |
| Melanoma Margin (Lung) | 33.9 | Normal Bladder | 8.0 |
| Normal Kidney | 8.0 | Bladder Cancer 1023 | 4.6 |
| Kidney Ca, Nuclear grade 2 | 19.3 | Bladder Cancer A302173 | 3.3 |
| (OD04338) | | | |
| Kidney Margin (OD04338) | 54.7 | Normal Stomach | 32.5 |
| Kidney Ca Nuclear grade ½ | 100.0 | Gastric Cancer 9060397 | 0.0 |
| (OD04339) | | | |
| Kidney Margin (OD04339) | 18.3 | Stomach Margin 9060396 | 6.1 |
| Kidney Ca, Clear cell type | 6.7 | Gastric Cancer 9060395 | 6.9 |
| (OD04340) | | | |
| Kidney Margin (OD04340) | 9.5 | Stomach Margin 9060394 | 22.2 |
| Kidney Ca, Nuclear grade 3 | 8.7 | Gastric Cancer 064005 | 6.1 |
| (OD04348) | | | |

TABLE IE
Panel 4D

| | Rel. Exp.(%) Ag3240, | | Rel. Exp.(%) Ag3240, |
|---|---|---|---|
| Tissue Name | Run 164389761 | Tissue Name | Run 164389761 |
| Secondary Th1 act | 0.0 | HUVEC IL-1beta | 16.7 |
| Secondary Th2 act | 0.0 | HUVEC IFN gamma | 33.2 |
| Secondary Tr1 act | 0.0 | HUVEC TNF alpha + IFN | 13.4 |
| | | gamma | |
| Secondary Th1 rest | 0.0 | HUVEC TNF alpha + IL4 | 17.1 |
| Secondary Th2 rest | 0.0 | HUVEC IL-11 | 13.5 |
| Secondary Tr1 rest | 0.0 | Lung Microvascular EC none | 25.9 |
| Primary Th1 act | 0.0 | Lung Microvascular EC | 23.5 |
| | | TNF alpha + IL-1beta | |
| Primary Th2 act | 0.0 | Microvascular Dermal EC none | 35.6 |
| Primary Tr1 act | 0.0 | Microsvasular Dermal EC | 21.6 |
| | | TNF alpha + IL-1beta | |
| Primary Th1 rest | 0.0 | Bronchial epithelium TNF alpha + IL1beta | 27.7 |
| Primary Th2 rest | 0.4 | Small airway epithelium none | 9.4 |
| Primary Tr1 rest | 0.0 | Small airway epithelium | 27.0 |
| | | TNF alpha + IL-1beta | |
| CD45RA CD4 lymphocyte | 8.5 | Coronery artery SMC rest | 31.6 |
| act | | | |
| CD45RO CD4 lymphocyte | 0.2 | Coronery artery SMC TNF alpha + IL- | 11.0 |
| act | | 1beta | |
| CD8 lymphocyte act | 0.0 | Astrocytes rest | 35.4 |
| Secondary CD8 | 0.2 | Astrocytes TNF alpha + IL-1beta | 19.9 |
| lymphocyte rest | | | |
| Secondary CD8 | 0.0 | KU-812 (Basophil) rest | 2.9 |
| lymphocyte act | | | |
| CD4 lymphocyte none | 0.1 | KU-812 (Basophil) | 8.2 |
| | | PMA/ionomycin | |
| 2ry Th1/Th2/Tr1_anti- | 0.0 | CCD1106 (Keratinocytes) none | 3.5 |
| CD95 CH11 | | | |
| LAK cells rest | 1.5 | CCD1106 (Keratinocytes) | 0.9 |
| | | TNF alpha + IL-1beta | |
| LAK cells IL-2 | 0.9 | Liver cirrhosis | 6.5 |
| LAK cells IL-2 + IL-12 | 0.4 | Lupus kidney | 6.6 |
| LAK cells IL-2 + IFN | 1.9 | NCI-H292 none | 71.7 |
| gamma | | | |
| LAK cells IL-2 + IL-18 | 1.1 | NCI-H292 IL-4 | 55.9 |
| LAK cells | 0.6 | NCI-H292 IL-9 | 87.7 |
| PMA/ionomycin | | | |
| NK Cells IL-2 rest | 0.0 | NCI-H292 IL-13 | 28.7 |
| Two Way MLR 3 day | 1.6 | NCI-H292 IFN gamma | 43.2 |
| Two Way MLR 5 day | 0.4 | HPAEC none | 19.6 |
| Two Way MLR 7 day | 0.0 | HPAEC TNF alpha + IL-1beta | 15.4 |
| PBMC rest | 0.4 | Lung fibroblast none | 49.7 |
| PBMC PWM | 1.4 | Lung fibroblast TNF alpha + IL- | 16.8 |
| | | 1beta | |
| PBMC PHA-L | 0.2 | Lung fibroblast IL-4 | 84.7 |
| Ramos (B cell) none | 7.6 | Lung fibroblast IL-9 | 59.0 |
| Ramos (B cell) ionomycin | 26.6 | Lung fibroblast IL-13 | 55.9 |
| B lymphocytes PWM | 3.7 | Lung fibroblast IFN gamma | 100.0 |
| B lymphocytes CD40L | 3.9 | Dermal fibroblast CCD1070 rest | 51.4 |
| and IL-4 | | | |
| EOL-1 dbcAMP | 5.1 | Dermal fibroblast CCD1070 | 33.2 |
| | | TNF alpha | |
| EOL-1 dbcAMP | 5.7 | Dermal fibroblast CCD1070 IL- | 20.0 |
| PMA/ionomycin | | 1beta | |
| Dendritic cells none | 0.7 | Dermal fibroblast IFN gamma | 36.6 |
| Dendritic cells LPS | 1.2 | Dermal fibroblast IL-4 | 52.5 |
| Dendritic cells anti-CD40 | 0.7 | IBD Colitis 2 | 2.8 |
| Monocytes rest | 0.0 | IBD Crohn's | 2.6 |
| Monocytes LPS | 0.2 | Colon | 24.8 |
| Macrophages rest | 0.2 | Lung | 59.5 |
| Macrophages LPS | 1.2 | Thymus | 42.9 |
| HUVEC none | 20.4 | Kidney | 17.3 |
| HUVEC starved | 51.4 | | |

TABLE IF
Panel CNS_1

| | Rel. Exp.(%) Ag3240, Run | | Rel. Exp.(%) Ag3240, Run |
|---|---|---|---|
| Tissue Name | 171694589 | Tissue Name | 171694589 |
| BA4 Control | 13.0 | BA17 PSP | 28.7 |
| BA4 Control2 | 21.6 | BA17 PSP2 | 6.7 |
| BA4 Alzheimer's2 | 4.8 | Sub Nigra Control | 41.8 |
| BA4 Parkinson's | 59.9 | Sub Nigra Control2 | 36.9 |
| BA4 Parkinson's2 | 34.6 | Sub Nigra Alzheimer's2 | 18.7 |
| BA4 Huntington's | 18.8 | Sub Nigra Parkinson's2 | 58.2 |
| BA4 | 5.0 | Sub Nigra Huntington's | 85.3 |
| Huntington's2 | | | |
| BA4 PSP | 2.7 | Sub Nigra | 23.8 |
| | | Huntington's2 | |
| BA4 PSP2 | 23.3 | Sub Nigra PSP2 | 11.6 |
| BA4 Depression | 19.2 | Sub Nigra Depression | 13.9 |
| BA4 Depression2 | 17.4 | Sub Nigra Depression2 | 11.4 |
| BA7 Control | 27.0 | Glob Palladus Control | 22.8 |
| BA7 Control2 | 15.9 | Glob Palladus Control2 | 17.9 |
| BA7 Alzheimer's2 | 13.5 | Glob Palladus | 17.2 |
| | | Alzheimer's | |
| BA7 Parkinson's | 34.2 | Glob Palladus | 9.8 |
| | | Alzheimer's2 | |
| BA7 Parkinson's2 | 26.8 | Glob Palladus | 100.0 |
| | | Parkinson's | |
| BA7 Huntington's | 43.5 | Glob Palladus | 10.5 |
| | | Parkinson's2 | |
| BA7 | 40.9 | Glob Palladus PSP | 17.2 |
| Huntington's2 | | | |
| BA7 PSP | 48.3 | Glob Palladus PSP2 | 7.4 |
| BA7 PSP2 | 17.1 | Glob Palladus | 9.3 |
| | | Depression | |
| BA7 Depression | 27.0 | Temp Pole Control | 11.8 |
| BA9 Control | 11.1 | Temp Pole Control2 | 33.4 |
| BA9 Control2 | 39.5 | Temp Pole Alzheimer's | 1.5 |
| BA9 Alzheimer's | 4.7 | Temp Pole Alzheimer's2 | 5.7 |
| BA9 Alzheimer's2 | 18.7 | Temp Pole Parkinson's | 39.8 |
| BA9 Parkinson's | 26.8 | Temp Pole Parkinson's2 | 12.9 |
| BA9 Parkinson's2 | 41.5 | Temp Pole Huntington's | 19.6 |
| BA9 Huntington's | 54.7 | Temp Pole PSP | 6.8 |
| BA9 | 8.8 | Temp Pole PSP2 | 0.7 |
| Huntington's2 | | | |
| BA9 PSP | 24.5 | Temp Pole Depression2 | 10.9 |
| BA9 PSP2 | 7.1 | Cing Gyr Control | 42.3 |
| BA9 Depression | 6.1 | Cing Gyr Control2 | 20.9 |
| BA9 Depression2 | 5.0 | Cing Gyr Alzheimer's | 23.3 |
| BA17 Control | 40.3 | Cing Gyr Alzheimer's2 | 7.9 |
| BA17 Control2 | 26.1 | Cing Gyr Parkinson's | 38.2 |
| BA17 | 6.0 | Cing Gyr Parkinson's2 | 51.1 |
| Alzheimer's2 | | | |
| BA17 Parkinson's | 52.1 | Cing Gyr Huntington's | 59.0 |
| BA17 | 32.1 | Cing Gyr Huntington's2 | 19.6 |
| Parkinson's2 | | | |
| BA17 | 21.0 | Cing Gyr PSP | 51.1 |
| Huntington's | | | |
| BA17 | 15.4 | Cing Gyr PSP2 | 13.6 |
| Huntington's2 | | | |
| BA17 Depression | 25.7 | Cing Gyr Depression | 18.3 |
| BA17 Depression2 | 10.2 | Cing Gyr Depression2 | 15.0 |

CNS_neurodegeneration_v1.0 Summary: Ag3240 Highest expression of the CG57444-01 gene is seen in the hippocampus of a patient with Alzheimer's disease (CT=27.6). This panel suggests a slight upregulation of this gene in the AD temporal cortex that falls short of significance (p=0.051). Thus, this gene product may be a drug target for the treatment of Alzheimer's disease; specifically, it may be involved in compensatory synaptogenesis in response to neuronal loss.

Panel 1.3D Summary: Ag3240 The CG57444-01 gene, a cadherin homolog, is expressed in many samples in this panel, reflecting the widespread role of cadherins in cell-cell adhesion. Highest expression is seen in a brain cancer cell line (CT=28.6). Thus, expression of this gene could be used to differentiate between this sample and other samples on this panel. Overall, however, expression appears to be associated with normal tissues rather than cancer cell lines. This can be seen in the samples from normal ovarian and breast tissue, which show higher levels of expression than the cell lines derived from breast and ovarian cancers. Loss of function of the related E-cadherin protein has been described in many tumors, along with an increased invasiveness and a decreased prognosis of many carcinomas, including tumors of endocrine glands and their target systems (ref 1). Thus, this gene product might similarly be useful as a protein therapeutic to treat a variety of tumors.

In addition, this gene is moderately expressed in pituitary gland, adrenal gland, thyroid, pancreas, and fetal and adult skeletal muscle, heart and liver, again reflecting the widespread role of this cadherin homolog. This observation may suggest that this gene product plays a role in normal metabolic and neuroendocrine function and that disregulated expression of this gene may contribute to metabolic diseases (such as obesity and diabetes) or neuroendocrine disorders.

Expression of this gene is also high in many regions of the brain, including the amygdala, thalamus, cerebellum, cerebral cortex, hippocampus, and substantia nigra. Expression is also detected in the spinal cord. Cadherins can act as axon guidance and cell adhesion proteins, specifically during development and in the response to injury (ref 2). Manipulation of levels of this protein may be of use in inducing a compensatory synaptogenic response to neuronal death in Alzheimer's disease, Parkinson's disease, Huntington's disease, spinocerebellar ataxia, progressive supranuclear palsy, ALS, head trauma, stroke, or any other disease/condition associated with neuronal loss (Potter et al., Endocr. Rev. 20: 207-239, 1999; Ranscht, Int. J. Dev. Neurosci. 18: 643-651, 2000).

Panel 2.2 Summary: Ag3240 Highest expression of the CG57444-01 gene is seen in a kidney cancer (CT=29.7). Overall, however, expression appears to be associated with normal tissues, as seen in Panel 1.3D. Thus, higher levels of expression are seen in normal colon, ovary, lung and stomach, when compared to adjacent tumors. Thus, absence of expression of this gene could be used as a diagnostic marker of these cancers. Furthermore, therapeutic modulation of the expression or function of this gene could be effective in the treatment of colon, ovarian, lung and stomach cancers.

Panel 4D Summary: Ag3240 The CG57444-01 transcript is expressed in endothelium, fibroblasts and NCI-H292 cells. This gene is also expressed in normal colon, kidney, thymus, and lung. This transcript encodes a putative protocadherin family member. Protocadherins mediate cell:cell interactions and signalling. Since this gene product is expressed in endothelium, it may also be involved in normal blood cell recirculation through the tissues. Alternatively, the protein encoded by this gene may be important for regulating tissue or organ organization and normal function since it is expressed on fibroblasts and epithelium.

Panel CNS_{—}1 Summary: Ag3240 Results from this experiment confirm expression of the CG57444-01 gene in the brain. Please see Panel 1.3D for discussion of utility of this gene in the central nervous system.

J. NOV9: Kerratin 18

Expression of gene CG57484-01 was assessed using the primer-probe set Ag1597, described in Table JA. Results of the RTQ-PCR runs are shown in Table JB.

TABLE JA

Probe Name Ag1597

| | | | |
|---|---|---|---|
| Pri- | | | Start |
| mers | Sequences | Length | Position |

| | | | |
|---|---|---|---|
| For- | 5′-gactgaggagagcaccacagt-3′ | 21 | 810 |
| ward | (SEQ ID NO:63) | | |
| Probe | TET-5′- | | |
| | acactctgccaaggtcagagctgct-3′- | 25 | 840 |
| | TAMRA (SEQ ID NO:64) | | |
| Re- | 5′-gtctcagctccgttgtcatct-3′ | 21 | 866 |
| verse | (SEQ ID NO:65) | | |

TABLE JB
Panel 2D

| | Rel. Exp.(%) Ag1597, | | Rel. Exp.(%) Ag1597, |
|---|---|---|---|
| Tissue Name | Run 164988404 | Tissue Name | Run 164988404 |
| Normal Colon | 2.3 | Kidney Margin 8120608 | 0.0 |
| CC Well to Mod Diff | 0.0 | Kidney Cancer 8120613 | 0.0 |
| (ODO3866) | | | |
| CC Margin (ODO3866) | 0.0 | Kidney Margin 8120614 | 0.0 |
| CC Gr.2 rectosigmoid | 0.0 | Kidney Cancer 9010320 | 0.0 |
| (ODO3868) | | | |
| CC Margin (ODO3868) | 0.0 | Kidney Margin 9010321 | 0.0 |
| CC Mod Diff (ODO3920) | 0.0 | Normal Uterus | 0.0 |
| CC Margin (ODO3920) | 0.0 | Uterus Cancer 064011 | 9.3 |
| CC Gr.2 ascend colon | 0.0 | Normal Thyroid | 0.0 |
| (ODO3921) | | | |
| CC Margin (ODO3921) | 0.0 | Thyroid Cancer 064010 | 2.5 |
| CC from Partial Hepatectomy | 0.0 | Thyroid Cancer A302152 | 0.0 |
| (ODO4309) Mets | | | |
| Liver Margin (ODO4309) | 0.0 | Thyroid Margin A302153 | 0.0 |
| Colon mets to lung (OD04451- | 0.0 | Normal Breast | 2.4 |
| 01) | | | |
| Lung Margin (OD04451-02) | 1.4 | Breast Cancer (OD04566) | 3.5 |
| Normal Prostate 6546-1 | 10.0 | Breast Cancer (OD04590- | 0.0 |
| | | 01) | |
| Prostate Cancer (OD04410) | 7.7 | Breast Cancer Mets | 30.4 |
| | | (OD04590-03) | |
| Prostate Margin (OD04410) | 2.6 | Breast Cancer Metastasis | 74.7 |
| | | (OD04655-05) | |
| Prostate Cancer (OD04720-01) | 22.4 | Breast Cancer 064006 | 14.0 |
| Prostate Margin (OD04720-02) | 10.8 | Breast Cancer 1024 | 39.2 |
| Normal Lung 061010 | 9.3 | Breast Cancer 9100266 | 0.0 |
| Lung Met to Muscle | 7.7 | Breast Margin 9100265 | 3.1 |
| (ODO4286) | | | |
| Muscle Margin (ODO4286) | 0.0 | Breast Cancer A209073 | 23.2 |
| Lung Malignant Cancer | 21.0 | Breast Margin A209073 | 30.1 |
| (OD03126) | | | |
| Lung Margin (OD03126) | 6.1 | Normal Liver | 16.7 |
| Lung Cancer (OD04404) | 2.9 | Liver Cancer 064003 | 0.0 |
| Lung Margin (OD04404) | 0.0 | Liver Cancer 1025 | 27.0 |
| Lung Cancer (OD04565) | 0.0 | Liver Cancer 1026 | 0.0 |
| Lung Margin (OD04565) | 0.0 | Liver Cancer 6004-T | 0.0 |
| Lung Cancer (OD04237-01) | 100.0 | Liver Tissue 6004-N | 0.0 |
| Lung Margin (OD04237-02) | 10.9 | Liver Cancer 6005-T | 0.0 |
| Ocular Mel Met to Liver | 2.9 | Liver Tissue 6005-N | 0.0 |
| (ODO4310) | | | |
| Liver Margin (ODO4310) | 0.0 | Normal Bladder | 0.0 |
| Melanoma Mets to Lung | 2.0 | Bladder Cancer 1023 | 0.0 |
| (OD04321) | | | |
| Lung Margin (OD04321) | 0.0 | Bladder Cancer A302173 | 0.0 |
| Normal Kidney | 55.1 | Bladder Cancer | 0.0 |
| | | (OD04718-01) | |
| Kidney Ca, Nuclear grade 2 | 14.3 | Bladder Normal Adjacent | 0.0 |
| (OD04338) | | (OD04718-03) | |
| Kidney Margin (OD04338) | 0.0 | Normal Ovary | 0.0 |
| Kidney Ca Nuclear grade ½ | 0.0 | Ovarian Cancer 064008 | 0.0 |
| (OD04339) | | | |
| Kidney Margin (OD04339) | 0.0 | Ovarian Cancer | 0.0 |
| | | (OD04768-07) | |
| Kidney Ca, Clear cell type | 0.0 | Ovary Margin (OD04768- | 0.0 |
| (OD04340) | | 08) | |
| Kidney Margin (OD04340) | 0.0 | Normal Stomach | 13.4 |
| Kidney Ca, Nuclear grade 3 | 0.0 | Gastric Cancer 9060358 | 0.0 |
| (OD04348) | | | |
| Kidney Margin (OD04348) | 36.9 | Stomach Margin 9060359 | 0.0 |
| Kidney Cancer (OD04622-01) | 0.0 | Gastric Cancer 9060395 | 0.0 |
| Kidney Margin (OD04622-03) | 4.7 | Stomach Margin 9060394 | 3.0 |
| Kidney Cancer (OD04450-01) | 0.0 | Gastric Cancer 9060397 | 0.0 |
| Kidney Margin (OD04450-03) | 0.0 | Stomach Margin 9060396 | 0.0 |
| Kidney Cancer 8120607 | 0.0 | Gastric Cancer 064005 | 0.0 |

Panel 2D Summary: Ag1597 Expression of the CG57484-01 gene is limited to samples derived from breast cancer and lung cancer (CTs=33). Thus, expression of this gene could be used to differentiate between these samples and other samples on this panel and as a marker to detect the presence of breast and lung cancers. This gene encodes a protein that is homologous to keratin 18. Expression of keratin 18 has been shown in both lung and breast cancers. Therefore, therapeutic modulation of the expression or function of this gene may be effective in the treatment of breast and lung cancers (Chu et al., Selection of invasive and metastatic subpopulations from a human lung adenocarcinoma cell line. Am J Respir Cell Mol Biol 1997 September; 17 (3):353-60).

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

1. An isolated polypeptide comprising an amino acid sequence selected from the group consisting of:
(a) a mature form of an amino acid sequence of SEQ ID NO:10, 12, 14, 16, or 18;
(b) a variant of a mature form of an amino acid sequence of SEQ ID NO:10, 12, 14, 16, or 18, wherein one or more amino acid residues in said variant differs from the amino acid sequence of said mature form, provided that said variant differs in no more than 15% of the amino acid residues from the amino acid sequence of said mature form;
(c) an amino acid sequence of SEQ ID NO:10, 12, 14, 16, or 18; and
(d) a variant of an amino acid sequence of SEQ ID NO:10, 12, 14, 16, or 18, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence of SEQ ID NO:10, 12, 14, 16, or 18.

3. The polypeptide of claim 1, wherein the amino acid sequence of said variant comprises a conservative amino acid substitution.

4. An isolated nucleic acid molecule comprising a nucleic acid sequence encoding the polypeptide of claim 1, or the complement of said nucleic acid sequence.

5. The nucleic acid molecule of claim 4, wherein the nucleic acid molecule comprises the nucleotide sequence of a naturally-occurring allelic nucleic acid variant.

6. The nucleic acid molecule of claim 4, wherein the nucleic acid molecule differs by a single nucleotide from a nucleic acid sequence of SEQ ID NO:11, 13, 15, or 17.

7. The nucleic acid molecule of claim 4, wherein said nucleic acid molecule comprises a nucleotide sequence selected from the group consisting of
(a) a nucleotide sequence of SEQ ID NO:11, 13, 15, or 17;
(b) a nucleotide sequence differing by one or more nucleotides from a nucleotide sequence of SEQ ID NO:9, 11, 13, 15, or 17, provided that no more than 20% of the nucleotides differ from said nucleotide sequence.

8. A vector comprising the nucleic acid molecule of claim 7.

9. The vector of claim 8, further comprising a promoter operably-linked to said nucleic acid molecule.

10. A cell comprising the vector of claim 8.

11. An antibody that immunospecifically-binds to the polypeptide of claim 1.

12. A pharmaceutical composition comprising the polypeptide of claim 1 and a pharmaceutically-acceptable carrier.

13. A pharmaceutical composition comprising the nucleic acid molecule of claim 4 and a pharmaceutically-acceptable carrier.

14. A pharmaceutical composition comprising the antibody of claim 11 and a pharmaceutically-acceptable carrier.

15. A method of producing the polypeptide of claim 1, the method comprising culturing a cell under conditions that lead to expression of the polypeptide, wherein said cell comprises the vector of claim 8.

16. An isolated polypeptide comprising
an amino acid sequence that is at least 95% identical to the amino acid sequence of SEQ ID NO: 10, 12, 14, 16, or 18.

17. The isolated polypeptide of claim 16 comprising amino acid sequence of SEQ ID NO: 10, 12, 14, 16, or 18.

18. The isolated polypeptide of claim 16 consisting of amino acid sequence of SEQ ID NO: 10, 12, 14, 16, or 18.

19. (canceled)


SEQUENCE LISTING (71 sequences)

SEQ ID NO: 1 - DNA, 872 bp, Homo sapiens
  CDS (11)..(844)
ctcgtgaaggatggtacgtgatgctcttgtttcccttgccgataggaagttctttgctggtggagtgtgtagaacaaatgtgcagcttcctggcaaggtagtggtgatcactggcgccaacacgggcattggcaaggagacggccagagagctcgctagccgaggagcccgagtctatattgcctgcagagatgtactgaagggggagtctgctgccagtgaaatccgagtggatacaaagaactcccaggtgctggtgcggaaattggacctatccgacaccaaatctatccgagcctttgctgagggctttctggcagaggaaaagcagctccatattctgatcaacaatgcgggagtaatgatgtgtccatattccaagacagctgatggctttgaaacccacctgggagtcaaccacctgggccacttcctcctcacctacctgctcctggagcggctaaaggtgtctgcccctgcacgggtggttaatgtgtcctcggtggctcaccacattggcaagattcccttccacgacctccagagcgagaagcgctacagcaggggttttgcctattgccacagcaagctggccaatgtgctttttactcgtgagctggccaagaggctccaaggcaccggggtcaccacctacgcagtgcacccaggcgtcgtccgctctgagctggtccggcactcctccctgctctgcctgctctggcggctcttctccccctttgtcaagacggcacgggagggggcgcagaccagcctgcactgcgccctggctgagggcctggagcccctgagtggcaagtacttcggtgtctccaagggcccgaaataacaaaacagctgagcgcctatggaat

SEQ ID NO: 2 - protein, 278 aa, Homo sapiens
MVRDALVSLADRKFFAGGVCRTNVQLPGKVVVITGANTGIGKETARELASRGARVYIACRDVLKGESAASEIRVDTKNSQVLVRKLDLSDTKSIRAFAEGFLAEEKQLHILINNAGVMMCPYSKTADGFETHLGVNHLGHFLLTYLLLERLKVSAPARVVNVSSVAHHIGKIPFHDLQSEKRYSRGFAYCHSKLANVLFTRELAKRLQGTGVTTYAVHPGVVRSELVRHSSLLCLLWRLFSPFVKTAREGAQTSLHCALAEGLEPLSGKYFGVSKGPK

SEQ ID NO: 3 - DNA, 4321 bp, Homo sapiens
  CDS (58)..(3114)
ccggctcgaagcgcaacgaggaagcgtttgcggtgatcccggcgactgcgctggctaatgcggtaccggctagcgtggcttctgcaccccgcactgcccagcaccttccgctcagtcctcggcgcccgcctgccgcctccggagcgcctgtgtggtttccaaaaaaagacttacagcaaaatgaataatccagccatcaagagaataggaaatcacattaccaagtctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaagttacgtgcagaaggacctcaagaatgggttttccaagagtgcaaggacttgaatgctgttgcttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagctatgagcaaactttggttcaaacaagatgataagttttttttgccgaaggcttgtctcaactttgaatttttcagtcgctacatttatgctgatcctctccattgcaacatgacatacctgtttatcaggttattgaaggatgatttaaaagagtatacatatgcagcacgcctctcaggtttgagctatggcattgcatcaggaatgaatgcaatacttctttcagtgaaaggttacaatgacaagcagccaattttactaaagaagattattgagaaaatggctacctttgagattgatgaaaaaagatttgaaattatcaaagaagcatatatgcgatctcttaacaatttccgggctgaacagcctcaccagcatgccatgtactacctccgcttgctgatgactgaagtggcctggactaaagatgagttaaaagaagctctggatgatgtaacccttcctcgccttaaggccttcatacctcagctcctgtcacggctgcacattgaagcccttctccatggaaacataacaaagcaggctgcattaggaattatgcagatggttgaagacaccctcattgaacatgctcataccaaacctctccttccaagtcagctggttcggtatagagaagttcagctccctgacagaggatggtttgtttatcagcagagagatgaagttcacaataactgtggcatcgagatatactaccaaacagacatgcaaagcacctcagagaatatgtttctggagctcttctgtcagattatctcggaaccttgcttcaacaccctgcgcaccaaggagcagttgggctatatcgtcttcagcgggccacgtcgagctaatggcatacagggcttgagattcatcatccagtcagaaaagccacctcactacctagaaagcagagtggaagctttcttaattaccatggaaaagtccatagaggacatgacagaagaggccttccaaaaacacattcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatgggaaagtgcaagtggatgcattcctgagtcttccagagcctaagaaaatcatcttggccactttaatagtttctgattcactattagagaaacaaacaaaaaattgtcaaatgtcattatgtagaaatattataaatccaaagtaaattacaaaatcttatagatgtagaatattttttaaatacatgcctcttaaatattttaaaatttttcttttgattactgagagaaatttccccaatataacaatgcttaaaatgaatgatattcctatagaatcttccttccctattctgtaaaatagtcacttgtccgaagaaagttaaaagttagctcttttctaaaagcctcctagcttgacatagaaggcttcacaacatttagaaaggtaataactttttaaaaattgatcctcaaatttgctttctacttgatggtttcatgtaaatcagtggaaaacattacatttggcagatgataaagcaatgtcatcttttattagtgaaatgctggttatataaggcatggttttaatctttttataaaatttgaacatgttttttatgccaactcgtaaaatgctagaaaaccctacttatttacaatgctagaaatacagacttaccttacatcaattttgtcctaaaccgaatttctcaggattactgtggtttctttcattctgattgaattatattgacctacttcttcatagttggtttgcagtgttccatgagttttacttttcctcatcaacatattgctttaacacaacatatttatttaacacgtacaaatagggtcaacttcagatcctactgagtgtgtgacatgcttttccaacatcagctttttgtaaccacctgtataactttttattacagtgaaattgcagtcagtatgtgaaccaaaatatcttgcccctttatgaatttaaaaggcagccaatacaaagccacctttttggaaatataaaaagtaaagccttgcattcttatatagcaggtcttcataaaactctaaaatcccttgttgctaccagtctaatcttgccttaaatgttaagttattttttgaatatataaatataaacatataaacacagatgatgactggagtagacttttaaaaaaatatttttttcatgagatactattttaggtgaaattgtactgtagatttacagct

SEQ ID NO: 4 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSRYIYADPLHCNMTYLFIRLLKDDLKEYTYAARLSGLSYGIASGMNAILLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRDEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 5 - DNA, 3153 bp, Homo sapiens
  CDS (37)..(3093)
aagcgtttgcggtgatcccggcgactgcgctggctaatgcggtaccggctagcgtggcttctgcaccccgcactgcccagcaccttccgctcagtcctcggcgcccgcctgccgcctccggagcgcctgtgtggtttccaaaaaaagacttacagcaaaatgaataatccagccatcaagagaataggaaatcacattaccaagcctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactccttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaggttacgtgcagaaggacctcaagaatgggttttccaagagtgcaaggacttgaatgctgttacttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagctatgagcaaactttggttcaaacaagatgataagttttttttgccgaaggcttgtctcaactttgaatttttcagtcgctacatttatgctgatcctctccattgcaacatgacatacctgtttatcaggttattgaaggatgatttaaaagagtatacatatgcagcacgcctctcaggtttgagctatggcattgcatcaggaatgaatgcaatacttctttcagtgaaaggttacaatgacaagcagccaattttactaaagaagattattgagaaaatggctacctttgagattgatgaaaaaagatttgaaattatcaaagaagcatatatgcgatctcttaacaatttccgggctgaacagcctcaccagcatgccatgtactacctccgcttgctgatgactgaagtggcctggactaaagatgagttaaaagaggctctggatgatgtaacccttcctcgccttaaggccttcatacctcagctcctgtcacggctgcacattgaagcccttctccatggaaacataacaaagcaggctgcattaggaattatgcagatggttgaagacaccctcattgaacatgctcataccaaacctctccttccaagtcagctggttcggtatagagaagttcagctccctgacagaggatggtttgtttatcagcagagaaatgaagttcacaataactgtggcatcgagatatactaccaaacagacatgcaaagcacctcagagaatatgtttctggagctcttctgtcagattatctcggaaccttgcttcaacaccctgcgcaccaaggagcagttgggctatatcgtcttcagcgggccacgtcgagctaatggcatacagggcttgagattcatcatccagtcagaaaagccacctcactacctagaaagcagagtggaagctttcttaattaccatggaaaagtccatagaggacatgacagaagaggccttccaaaaacacattcaggcattagcaattcgtcgactagacaaaccaaagaagctatctgctgagtgtgctaaatactggggagaaatcatctcccagcaatataattttgacagagataacactgaggtagcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatgggaaagtgcaagtggatgcattcctgagtcttccagagcct

SEQ ID NO: 6 - protein, 1019 aa, Homo sapiens
MRYRLAWLLHPALPSTFRSVLGARLPPPERLCGFQKKTYSKMNNPAIKRIGNHITKPPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLLQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQRLRAEGPQEWVFQECKDLNAVTFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSRYIYADPLHCNMTYLFIRLLKDDLKEYTYAARLSGLSYGIASGMNAILLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 7 - DNA, 3509 bp, Homo sapiens
  CDS (155)..(3205)
ggtccgagcggggctctgtgacgacagccccaccagctgcttcggggtgggcagccttcaggaggaagggctggcctgggaggagctggctgcccctcgggacgtgttctcaggccctgcccgctgccctgccccatataccttctccttcgagatgctggtgactgggccatgcctgcttgcaggcctggagagcccctctcatgctctgcgggcagatgccccccctcatgccagctctgcagccaccatctgtgtcacactggcagagggccaccactgtgaccgggccttggagatcctgctgcaccccagtgagccccatcagccacacctgatgctggagggcggcagcctgagctcagcagaatatgaggcccgggtgagggcccgccgagattttcagaggctacagcgaggggacagtgatggggaccggcaggtgtggttcctgcagcgacgcttccacaaggacatcctgctgaaccccgtgctggcgctgagcttctgcccagacctgagctccaagcccggacacctggggacagctactcgggagctactcttccttttggatagcagcagcgtggcacacaaggatgccattgttttggctgtgaagtccctcccaccccagacgcttatcaacctggccgtgtttgggacgttggtgcagccactcttcccagagagccggccttgcagtgatgatgctgtgcagctgatctgcgagagcattgagaccctgcaggttccgagtgggcccccagacgtgctggctgctctggactgggccgtggggcagccccagcacagggcctaccctcggcagctgttcctgctcactgctgcctcacccatggccgccactacccaccgaaccctggagctcatgaggtggcacagggggacagccaggtgcttctcctttgggctggggcccacctgccaccagctgctccagggtttatctgccctcagcagccgaggtcgcgccactgcactccagcctgggcgacagagcaagccacagctggtacaggctctgcggaaggcactggagcctgctttgagtgacatctctgtggactggtttgtgcccgacactgtggaggcactgctgacccctcgggagatcccagcactctaccctggggaccagctgctcggttactgctcactcttcagggtggatggcttccggtcccgcccaccagggggccaagagcctggctggcagagctcgggtgggtccgtgtttccatccccagaagaggccccgtctgctgccagccctggcactgagcccactggcacctcagagccactgggaacaggcactgtctcagcagaactgtccagcccatgggctgccagggactcggagcagagtggtactgatgctctgacagacccagtcacggatcctggacccaacccctctgacacagccatatggcgccgcatctttcagtcctcgtacattcgggagcagtatgtgctcacccactgctctgccagccccgagccaggcccaggctccacaggcagcagtgagtccccaggctcacagggccctggctcccccgaaggtagtgctcccttggagcccccttctcagcagggctgccgcagtctggcctggggagaacctgcaggctcccgctcctgtcccctgcctgcacccacaccagctccattcaaggtgggggccttgagtactgaggtgctgggccgtcagcacagagcggctctggctggccgaagcctctcatcccctccaggccgggcaaaccaagtccccggccgaccccggaaaccctctttgggtgcaatactagatggcccaagtcctgagccaggccaacagttgggacaaggcctggatgactcaggtaacctgctctccccagcccctatggactgggacatgctgatggaaccacccttcttattcacggctgtgcctcctagtggggagttggcccctccagcagtgcctccccaggctccacgctgccatgtggtgatccggggcctgtgtggggagcagcccatgtgctgggaggtgggtgttgggctggagacactgtggggacctggagatggctcacagcctccctcacctcctgtaagagaagctgcttgggaccaagcactccatcggctgacagcagcctctgtggtccgggacaatgagcagctggccctccgaggaggggcagagaccacagctgaccggggccatgcccggaggtgctggcttcgagcccttcaaacaagtaaggtcagctctgccccctcctgcttcacttgccctgtagctgtggatgctactactagggaggtcctgcctggggccctgcaggtgtgcagctcagagcccgctgagcccccaggaacccctcctgcctctcacagccatctagatgcagctcctctgcccactgttgtctactctaaaggtgcctgggactcggaccaaaatggcaactccaagcgtgctttgggggaccctgccactcccacggaaggtcctcgccgcccacctccccgtcctccctgtcggctcagcatgggccgccgtcacaaactctgtagccctgacccgggccaggccaacaacagtgaaggcagcgaccatgactacctgcccttggtgcggctgcaggaggcaccaggctccttccgcctggacgcgcccttctgcgccgctgtgcgcatctcgcaggagcgcctctgccgtgcctcgccctttgccgtgcaccgcgccagcctcagccccacctcggcctcattgccctgggcacttctgggccctggtgttggccagggtgacagtgccacggcctcctgcagcccgtcccccagctcgggctctgaggggccaggccaggtggacagtgggcggggctcagacaccgaggcctccgagggggcggaagggctgggcggcaccgacctgcggggccggacctgggccactgccgtagcactcgcctggctggagcaccgatgcgccgctgccttcgacgagtgggaactgacagcggccaaggctgattgctggctgcgggcccagcacttgcctgacggccttgacctggccgccctcaaggccgcagcccgagggctcttcctgctactgcgccactgggaccaaaacctgcagctacacctgctgtgctacagcccagcgaacgtgtgaaggctgccccctgctgcttgggctggcgccccacccaacacactcaagtcactgccgcccagggctggcctcttggtgctgggaaagtgtaggctggttccagcctgtcccccactgcttcttactccctccctagagccctcttgcccccacaaaaagtgcctgcctgtgctctctccctctcctcccaccccactcacactcccctccatcctttgagctccctgcaacacagtggaagggtagagagccacagtccccaaatcctatgcaataaagtgcctcttagggaaaaaaaaaa

SEQ ID NO: 8 - protein, 1017 aa, Homo sapiens
MLVTGPCLLAGLESPSHALRADAPPHASSAATICVTLAEGHHCDRALEILLHPSEPHQPHLMLEGGSLSSAEYEARVRARRDFQRLQRGDSDGDRQVWFLQRRFHKDILLNPVLALSFCPDLSSKPGHLGTATRELLFLLDSSSVAHKDAIVLAVKSLPPQTLINLAVFGTLVQPLFPESRPCSDDAVQLICESIETLQVPSGPPDVLAALDWAVGQPQHRAYPRQLFLLTAASPMAATTHRTLELMRWHRGTARCFSFGLGPTCHQLLQGLSALSSRGRATALQPGRQSKPQLVQALRKALEPALSDISVDWFVPDTVEALLTPREIPALYPGDQLLGYCSLFRVDGFRSRPPGGQEPGWQSSGGSVFPSPEEAPSAASPGTEPTGTSEPLGTGTVSAELSSPWAARDSEQSGTDALTDPVTDPGPNPSDTAIWRRIFQSSYIREQYVLTHCSASPEPGPGSTGSSESPGSQGPGSPEGSAPLEPPSQQGCRSLAWGEPAGSRSCPLPAPTPAPFKVGALSTEVLGRQHRAALAGRSLSSPPGRANQVPGRPRKPSLGAILDGPSPEPGQQLGQGLDDSGNLLSPAPMDWDMLMEPPFLFTAVPPSGELAPPAVPPQAPRCHVVIRGLCGEQPMCWEVGVGLETLWGPGDGSQPPSPPVREAAWDQALHRLTAASVVRDNEQLALRGGAETTADRGHARRCWLRALQTSKVSSAPSCFTCPVAVDATTREVLPGALQVCSSEPAEPPGTPPASHSHLDAAPLPTVVYSKGAWDSDQNGNSKRALGDPATPTEGPRRPPPRPPCRLSMGRRHKLCSPDPGQANNSEGSDHDYLPLVRLQEAPGSFRLDAPFCAAVRISQERLCRASPFAVHRASLSPTSASLPWALLGPGVGQGDSATASCSPSPSSGSEGPGQVDSGRGSDTEASEGAEGLGGTDLRGRTWATAVALAWLEHRCAAAFDEWELTAAKADCWLRAQHLPDGLDLAALKAAARGLFLLLRHWDQNLQLHLLCYSPANV

SEQ ID NO: 9 - DNA, 468 bp, Homo sapiens
  CDS (31)..(438)
tgaagtttgtctgtgcactagcaccctggaatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcttaagtctgagcaagaaaagaagaaaagaaaaaaacaagctctccctcctctcctgccagaaggacgtcggattccccctgctgaaacttacaagtgccgttgtggactcccagatctcttgctgcaagggagacctctgcaatgcggtggtcctggcagccagcagcccctgggccctgtgcgtacagctcctgctcagcctggggtcagtcttcctctgggccctgctgtgagggccctttcccgccctctcccccgca

SEQ ID NO: 10 - protein, 136 aa, Homo sapiens
MSSLQAMKTLSLVLLVALLSMERAQGLRCYRCLAVLEGASCSVVSCPFLDGVCVSQKVSLSLSKKRRKEKNKLSLLSCQKDVGFPLLKLTSAVVDSQISCCKGDLCNAVVLAASSPWALCVQLLLSLGSVFLWALL

SEQ ID NO: 11 - DNA, 610 bp, Homo sapiens
  CDS (47)..(514)
cttgtaagggcgagacagcagagactggcaccagggaggctcctccatgggagtcttccatgattactcacagcgggtgggcagaggtgttgctaggaagcatgttctgggggggtcctctgggtgcacacgtgcagtagctgcacctgcttgctcatacgtcgcatgtgtcattagcatcttaaatctccaccaggggtgtgttttcttgccctctctcccagctcagggtctgcgctgctacagatgcttggcggtcttggaaggggcctcctgcagcgtggtctcgtgccccttcctggatggggtctgtgtctcccagaaagtgagcgttttggcagtgagtccctggggtgccagggcagagggcaggttaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgcctcactgaattgcag

SEQ ID NO: 12 - protein, 156 aa, Homo sapiens
MGVFHDYSQRVGRGVARKHVLGGSSGCTRAVAAPACSYVACVISILNLHQGCVFLPSLPAQGLRCYRCLAVLEGASCSVVSCPFLDGVCVSQKVSVLAVSPWGARAEGRLSAVVDSQISCCKGDLCNAVVLAAGSPWALCVQLLLSLGSVFLWALL

SEQ ID NO: 13 - DNA, 406 bp, Homo sapiens
  CDS (2)..(403)
aatgagcagtctccaggccatgaagaccttgtccctggtcctgctggtggccctgctgagcatggagagagctcagggtctgcgctgctacagatgcttggcggtcttggaaggggcctcctgcagcgtggtctcgtgccccttcctggatggggtctgtgtctcccagaaagtgagcgtctttggcagtaaagtgagaggggagaacaagctctccctcctctcctgccagaaggacgtcggattccccctgctgaaacttacaagtgccgttgtggactcccagatctcttgctgcaagggagacctctgcaatgcggtggtcctggcagccggcagcccccgggccctgtgcgtacagctcctgctcagcctggggtcagtcttcctctgggccctgctgtga

SEQ ID NO: 14 - protein, 134 aa, Homo sapiens
MSSLQAMKTLSLVLLVALLSMERAQGLRCYRCLAVLEGASCSVVSCPFLDGVCVSQKVSVFGSKVRGENKLSLLSCQKDVGFPLLKLTSAVVDSQISCCKGDLCNAVVLAAGSPRALCVQLLLSLGSVFLWALL

SEQ ID NO: 15 - DNA, 611 bp, Homo sapiens
  CDS (47)..(601)
cttgtaagggcgagacagcagagactggcaccagggaggctcctccatgggagtcttccatgattactcacagcgggtgggcagaggtgttgctaggaagcatgttctgggggggtcctctgggtgcacacgtgcagtagctgcacctgcttgctcatacgtcgcatgtgtcattagcatcttaaatctccaccaggggtgtgttttcttgccctctctcccagctcagggtctgcgctgctacagatgcttggcggtcttggaaggggcctcctgcagcgtggtctcgtgccccttcctggatggggtctgtgtctcccagaaagtgagcgtctttggcagtgagtccctggggtgccagggcagagggcaggttaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgcctcactgaattgcag

SEQ ID NO: 16 - protein, 185 aa, Homo sapiens
MGVFHDYSQRVGRGVARKHVLGGSSGCTRAVAAPACSYVACVISILNLHQGCVFLPSLPAQGLRCYRCLAVLEGASCSVVSCPFLDGVCVSQKVSVFGSESLGCQGRGQVKCRCGLPDLLLQGRPLQCGGPGSRQPLGPVRTAPAQPGVSLPLGPAVRAFPALSPAGLPSVPVRHQLLGFEELPH

SEQ ID NO: 17 - DNA, 423 bp, Homo sapiens
  CDS (1)..(423)
ggatcccaccaggggtgtgttttcttgccctctctcccagctcagggtctgcgctgctacagatgcttggcggtcttggaaggggcctcctgcagcgtggtctcgtgccccttcctggatggggtctgtgtctcccagaaagtgagcgtctttggcagtgagtccctggggtgccagggcagagggcaggttaagtgccgttgtggactcccagatctcttgctgcaagggagacctctgcaatgcggtggtcctggcagccggcagcccctgggccctgtgcgtacagctcctgctcagcctggggtcagtcttcctctgggccctgctgtgagggcctttcccgccctctcccccgcaggcctaccctctgtccctgtgcgtcaccagctgcttggttttgaagagctgcctcacctcgag

SEQ ID NO: 18 - protein, 141 aa, Homo sapiens
GSHQGCVFLPSLPAQGLRCYRCLAVLEGASCSVVSCPFLDGVCVSQKVSVFGSESLGCQGRGQVKCRCGLPDLLLQGRPLQCGGPGSRQPLGPVRTAPAQPGVSLPLGPAVRAFPALSPAGLPSVPVRHQLLGFEELPHLE

SEQ ID NO: 19 - DNA, 1260 bp, Homo sapiens
  CDS (107)..(1162)
ctcaagcaggcgtttgcgagaggagatacgagctggacgcctggcccttccctcccaccgggtcctagtccaccgctcccggcgccggctccccgcctctcccgctatgtaccgaccgcgagcccgggcggctcccgagggcagggtccggggctgcgcggtgcccggcaccgtgctcctgctgctcgcctacctggcttacctggcgctgggcaccggcgtgttctggacgctggagggccgcgcggcgcaggactccagccgcagcttccagcgcgacaagtgggagctgttgcagaacttcacgtgtctggaccgcccggcgctggactcgctgatccgggatgtcgtccaagcatacaaaaacggagccagcctcctcagcaacaccaccagcatggggcgctgggagctcgtgggctccttcttcttttctgtgtccaccatcaccaccattggctatggcaacctgagccccaacacgatggctgcccgcctcttctgcatcttctttgcccttgtggggatcccactcaacctcgtggtgctcaaccgactggggcatctcatgcagcagggagtaaaccactgggccagcaggctggggggcacctggcaggatcctgacaaggcgcggtggctggcgggctctggcgccctcctctcgggcctcctgctcttcctgctgctgccaccgctgctcttctcccacatggagggctggagctacacagagggcttctacttcgccttcatcaccctcagcaccgtgggcttcggcgactacgtgattggaatgaacccctcccagaggtacccactgtggtacaagaacatggtgtccctgtggatcctctttgggatggcatggctggccttgatcatcaaactcatcctctcccagctggagacaaacccatcctctcccagctggagacaacagggagggtatgttcctgctgccaccacagctctaaggaagacttcaagtcccaaagctggagacagggacctgaccgggagccagagtcccactccccacagcaaggatgctatccagagggacccatgggaatcatacagcatctggaaccttctgctcacgctgcaggctgtggcaaggacagctagttatactccattctttggtcgtcgtcctcggtagcaagacccctgattttaagctttgcacatgtccacccaaactaaagactacattttccatccaccctagaggctgggtgcagctatatgattaat

SEQ ID NO: 20 - protein, 352 aa, Homo sapiens
MYRPRARAAPEGRVRGCAVPGTVLLLLAYLAYLALGTGVFWTLEGRAAQDSSRSFQRDKWELLQNFTCLDRPALDSLIRDVVQAYKNGASLLSNTTSMGRWELVGSFFFSVSTITTIGYGNLSPNTMAARLFCIFFALVGIPLNLVVLNRLGHLMQQGVNHWASRLGGTWQDPDKARWLAGSGALLSGLLLFLLLPPLLFSHMEGWSYTEGFYFAFITLSTVGFGDYVIGMNPSQRYPLWYKNMVSLWILFGMAWLALIIKLILSQLETNPSSPSWRQQGGYVPAATTALRKTSSPKAGDRDLTGSQSPTPHSKDAIQRDPWESYSIWNLLLTLQAVARTASYTPFFGRRPR

SEQ ID NO: 21 - DNA, 627 bp, Homo sapiens
  CDS (31)..(597)
tctgctgactctcaacttttcttggaaaaaatggatcattcccatcatacggggatgagctgtatggactccaacagtaccatgcaacgtcctcaccatcacccaaccacttcagcctcacactcccgtggtggaggagacagcaacatgatgatgatgatgcctatgaccttctactttggctttaagaatgtggaactactgttttccagtttggtgatcaatacagctggagaaatggctggagcttttgtggcagtgcttttgctacaattccatgcctgtcccatagcccaagagagcctgctgtgtaagtcacaagtcagcttttgctacaattccatgcctgtcccaggaccaaatggaaccatccttatggagacacacaaaactgttgggcagcagatgctgagctttcctcacctcctgcaaacagtgctgcacatcatccaggtggtcataagctacctcctcatgctcatcttcatgacctacaatgggtacctctgcattgcagtaacagcaggggccggtacaagatacttcctcttcagctggaagaaggcagtggtagtggacatcacagagtattgccattgacgtcaaactctatggcatggccttatc

SEQ ID NO: 22 - protein, 189 aa, Homo sapiens
MDHSHHTGMSCMDSNSTMQRPHHHPTTSASHSRGGGDSNMMMMMPMTFYFGFKNVELLFSSLVINTAGEMAGAFVAVLLLQFHACPIAQESLLCKSQVSFCYNSMPVPGPNGTILMETHKTVGQQMLSFPHLLQTVLHIIQVVISYLLMLIFMTYNGYLCIAVTAGAGTRYFLFSWKKAVVVDITEYCH

SEQ ID NO: 23 - DNA, 1478 bp, Homo sapiens
  CDS (21)..(1463)
actccactcctgcctccaccatgtccatcagggtgacccagaagtcctacaaggtgtccacctctggcccccaggcctttagcagccgcttttacacgaatgggcctggtgcccacatcagctcttcaagcctctcccgagtgagcagcagcagcttccggggtggcctgggcagaggctatggtggggccagcggcataggaggcatcaccactgtcacgttcaaccagagcctgctgagccctgttaacctggaggtagatcccaatatccaggccatgcacacccaggagaaggagcagatcaagaccctcaacaacaagtttgcctccttcatcgacaaggtacggttcctgcagcagaagaacaagatgctggagaccaagtggagcctcctgcagcagcagaagatggctcggatcaacgtatttgagagctacatgaacaaccttaggcggcagctggaggctctgggccaggagaagctgaagctggaggcggagcttggcaatatgcaggggctggtggaggacttcaagaacaagtatgaggatgagatcaataagcgtacagaaacggagaatgaatttgtcctcatcaagaaggacatggatgaagcttacatgaacaaggcagagctggagtctcgcctggaagggctgactgacgagatcaacttcctcaggcaactgcatgaagaggagatccaggagctgcagtcccagatctcgggcacgtctgcggtgctgtccatggacaacagcctctccctggacatggacagcatcatcgctgaggtcaaggcacaggaggaggagatcgccaaccgcagctgggctgaggctgagagcatgtaccagatcaagtatgcagagctgcagacgctggctggcaagcacggggatgacctgcggtgtacaaagactaagatctccgagatgaaccggaacatcagccggctccaggctgagattgagggcctcaaaggccagagggcttccctggaggcccccatcgcagataccgagcagcgtggggagctggccgttaaggatgccagcgccaagcggtcggagctggaggccgccctgcagcgggccaagcaggacatggcgcagcagctgcgtgagtaccaggagctgatgaacgtcaaactggccctggacatggagatcgccacctacaggaagctgctggagggcgaggagagcgcccggctggagtctgggatgcagaacacgagtatccataggaagaccaccagcggctatgcaggtggtctgagttccgcctatgggggcctcacaagccctggcctcagctatggcctaagctccagctttggctctgtcgcgggctccagctcattcagccgcaccggctccgccagggccatggttgtgcagaagattgagacccgcgatgggaagctggtatccgagtcctctgacgtcctgcccaagtgaagagctgcggca

SEQ ID NO: 24 - protein, 481 aa, Homo sapiens
MSIRVTQKSYKVSTSGPQAFSSRFYTNGPGAHISSSSLSRVSSSSFRGGLGRGYGGASGIGGITTVTFNQSLLSPVNLEVDPNIQAMHTQEKEQIKTLNNKFASFIDKVRFLQQKNKMLETKWSLLQQQKMARINVFESYMNNLRRQLEALGQEKLKLEAELGNMQGLVEDFKNKYEDEINKRTETENEFVLIKKDMDEAYMNKAELESRLEGLTDEINFLRQLHEEEIQELQSQISGTSAVLSMDNSLSLDMDSIIAEVKAQEEEIANRSWAEAESMYQIKYAELQTLAGKHGDDLRCTKTKISEMNRNISRLQAEIEGLKGQRASLEAPIADTEQRGELAVKDASAKRSELEAALQRAKQDMAQQLREYQELMNVKLALDMEIATYRKLLEGEESARLESGMQNTSIHRKTTSGYAGGLSSAYGGLTSPGLSYGLSSSFGSVAGSSSFSRTGSARAMVVQKIETRDGKLVSESSDVLPK

SEQ ID NO: 25 - DNA, 2801 bp, Homo sapiens
  CDS (6)..(2723)
accggatgcacccggagatctcctactcaattcctgaggaaagagagaaaggctctttcgtgggcaacatctccaaggacttgggtctggcgccccgggagctggcggagcgcggagtccgcatagtctccagaggtaggacgcagcttttctctctgaacccgcgcagcggcagcttggtcaccgcgggcaggatagaccgggaggagctctgcgctcagagcgcgcggtgcgtggtgagttttaatatccttgtggaagacagggtgaaactttttgggatagaaatagaagtaactgatatcaatgacaatgctccaaaattccaagcagaaaatctagacgtaaaaattaatgaaaatgtcgctgcgggaatgcgttttcctctcccggaagctattgatccggatataggtgtaaactccctgcagggttaccagctcaactcaaacggttacttttccctggacgtgcaaagtggggccgatgggattaagtacccagagctggtgctggaacgcgctctagatcgcgaggaagaggcggttcaccacctggtccttactgccatggatggcggcgaccctctccgctcaagcgtcgcccaaattctggtaacagttctagatgtgaatgacaacactccaatgtttactcagcctgtctaccgtgtaagtgttcctgaaaacctgccagctcccggaactcgggtgctgatggttaatgcaacggatccagatgaaggagtcaatgcggaagtaatgtattcatttcggaaagtcagagacgaaagagcacagctattacagttgttttatctgagtgcggagataacgataatgaggggtctggaggatgtggactatggatactatgacatagatgacatagacgatgaaggccatggtgtccgtgctagaagagcggtacgcaaggtagtggtggaagttttggatgaaaatgacaacgccccagaaatcacagtcacctccgtcaccaccgcagtccccgaagctgcttctggaactgccattattttcctcaatgatagtgaccgagaggacggggggaacagtccatttatcagttctgtcaatccgggtctttcattcaaaaaattagataaaaaagatgattatttcatttttaaaacgactcaagacatagaccgaaaaactgtgtccgaatacaacatcaccgcaatagccccagaaacccgagctccttccccttcaactcatattacactccttgtgctagtgatcgacatcaatgaaaaccctcccccttattctcaatcctcctactacgtttacgtaaacgaaaacaacggcgccggcacttcaattatgaccgttaatgactctgaccccgatgacaattctagtgttatttactccttggcagaggctacccaaggagctcctccctcctccacctatgcctctatcacatcaaacactggtgtgcttgatgtgttgtccttcttctactatgagtattttgattttctgcaaatgcagatgacggctaatgacagtggcagcccaccacttagcagtaatgcgtcaatgagattgtttgtgttggacaaggatgacaatgcccaagacctccagtaccctgccccccccactggtggtactgctgttgaggtgctgccccgctctgcagcgcctggctacctggtgaccaaggtggtggcagtggacggagactcaggccagaatgcttggctctcctaccgcctattcgaggccagtgagccggggctcttctcggtggggctgcacacaggtggaaaagtgcgcaccgctcgggccctgctagatagagatgcgctcaaacagagccttgtggtggctgtacaggaccatggccagccccctctctccgccgccaccctcactgtggcggttgccgacgactccctccaagacctcgcggatttcttcggcagcctcacgccttcagaacaccaagacgactccggcctcacactccttcttgtggtagtagtggctgcagtctgcttcgtcttcccggtcttcgtcgtcgtgctgctagtactcaagctgaggcgctggcacaagtcccgcctgcttcacgctgaaggcagcaggttggcaggggtggctgcctcccactttgggggcgtggagggggttggggttttcctgccgaactattcccacgaggtctccctcaccgcggactcgcggaagagccccctgatccccacccaaccccgcgctgaaattcccctcagcaaccgggagagtggagagagaagccgccgtctggtgatacttaaggatgtgcttgaaacagagggcgaccctagtggacagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgccacactgaccaacgcagctggcaagcgggatggcaaggccccagcaggtggcaatggcaacaagaagaagtcgggcaagaaggagaagaagtaacatggaggccaggccaagagccacagggcggcctctccccaaccagcccagcttctccttacctgcacccaggcc

SEQ ID NO: 26 - protein, 906 aa, Homo sapiens
MHPEISYSIPEEREKGSFVGNISKDLGLAPRELAERGVRIVSRGRTQLFSLNPRSGSLVTAGRIDREELCAQSARCVVSFNILVEDRVKLFGIEIEVTDINDNAPKFQAENLDVKINENVAAGMRFPLPEAIDPDIGVNSLQGYQLNSNGYFSLDVQSGADGIKYPELVLERALDREEEAVHHLVLTAMDGGDPLRSSVAQILVTVLDVNDNTPMFTQPVYRVSVPENLPAPGTRVLMVNATDPDEGVNAEVMYSFRKVRDERAQLLQLFYLSAEITIMRGLEDVDYGYYDIDDIDDEGHGVRARRAVRKVVVEVLDENDNAPEITVTSVTTAVPEAASGTAIIFLNDSDREDGGNSPFISSVNPGLSFKKLDKKDDYFIFKTTQDIDRKTVSEYNITAIAPETRAPSPSTHITLLVLVIDINENPPPYSQSSYYVYVNENNGAGTSIMTVNDSDPDDNSSVIYSLAEATQGAPPSSTYASITSNTGVLDVLSFFYYEYFDFLQMQMTANDSGSPPLSSNASMRLFVLDKDDNAQDLQYPAPPTGGTAVEVLPRSAAPGYLVTKVVAVDGDSGQNAWLSYRLFEASEPGLFSVGLHTGGKVRTARALLDRDALKQSLVVAVQDHGQPPLSAATLTVAVADDSLQDLADFFGSLTPSEHQDDSGLTLLLVVVVAAVCFVFPVFVVVLLVLKLRRWHKSRLLHAEGSRLAGVAASHFGGVEGVGVFLPNYSHEVSLTADSRKSPLIPTQPRAEIPLSNRESGERSRRLVILKDVLETEGDPSGQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 27 - DNA, 2836 bp, Homo sapiens
  CDS (15)..(2801)
aacccgagcgaacgatgggagggagctgcgcgcagaggcgccgggccggcccgcggcaggtactatttcctttgctgctgcctttgttctaccccacgctgtgtgagccgatccgctactcgattccggaggagctggccaagggctcggtggtggggaacctcgctaaggatctagggcttagtgtcctggatgtgtcggctcgcgagctgcgagtgagcgcggagaagctgcacttcagcgtagacgcgcagagcggggacttacttgtgaaggaccgaatagaccgtgagcaaatatgcaaagagagaagaagatgtgagttgcaattggaagctgtggtggaaaatcctttaaatatttttcatgtcattgtggtgattgaggatgttaatgaccacgcccctcaattccggaaagatgaaataaacttagaaatcagtgaatccgtcagcctggggatgggaacaattcttgagtctgcagaagatcctgatattagtatgaattcgctgagcaaataccaactaagtcctaacgagtatttctcattggtggagaaagacaatcctgatggtggcaaatatccagaattagtattgcagaagactctggaccgagaaacgcagagcgctcaccacttgatattgaccgccttggacggaggggacccaccaagaagtgccaccgctcacatagaaatttctgtcaaggataccaatgataaccccccggttttcagcagagacgaatatagaattagtcttagtgaaaatctgccccctgggtcccctgtgttgcaagtgacagccactgaccaggatgagggggtcaatgctgagataaactactacttccgaagcactgcccagagcacaaaacatatgttctcattggatgagaaaacaggtatgattaagaataaccagtcatttgattttgaagatgtagaaaggtacaccatggaagtggaagcgaaggacggaggtggtctctctacccagtgtaaagtaatcatagaaatccttgatgaaaacgacaacagcccagaaataatcatcacttctctctctgatcagattttggagaattcacctccaggaatggttgttgccctcttcaaaacacgggatctggatttcggaggaaatggagaagtcaggtgtaatatagaaacagacattccattcaagatttattcttcttccaataactactacaaactggtgacagatggagccctggaccgagagcagacaccagaatacaatgtcaccatcgtagccactgacaggggcaagccgcctctttcttccagtagaagcatcaccttgtatgtcgctgacatcaacgacaacgccccagttttcgaccagacgtcctacgtggtccacgtggccgagaacaacccgccaggagcctccattgcgcaagtgagcgcctctgacccggatttggggctcaatggccacatctcctactctctcattgccagcgacctggagtcacgaacgctgtcgtcctacgtgtccgtgagcgcgcagagcggggtggtgttcgcgcagcgcgccttcgaccacgagcagctgcgcgccttcgcgctcacgctgcaggcccgcgaccagggctcgcccgcgctcagcgcgaacgtgagcctgcgcgtgttagtggacgaccgcaacgacaatgcgccacgggtgctgtacccagctctgggtcctgacggctccgcgttcttcgatatggtacctcgctctgcagagcccggctacctagtgactaaggtggtagcggtggacgccgactcgggacacaacgcctggctgtcctaccacgtgctgcaggccagtgagcccgggctcttcagcctggggctgcgaacaggcgaggtgcgcatggtgcgtgctttgggtgacaaggactcggtccgccagcgcctgctagtcgctataagagatggaggacagccacccctttcagccactgccacgctgcacctggtgttcgcagatagcttgcaagaggtactgccggatttcagcgaccatcccacaccctctgactcccaggctgagatgcagttttacctggtggtggccttggccttgatttctgtgctctttctcctcgcggtgattctagctattgctctacgcctgcgacagtctttcagccctactgcaggagactgctttgagtcagttctctgctccaagtccggacctgtgggtccccccaactacagtgagggaacgttgccctatgcctataatttttgtgtgcctggggatcaaatgaatccagaatttaattttttcacatctgttgatcattgtccagccacacaagataacctcaacaaagatagcatgctactggctagcattttaactcccagcgttgaagcagataagaagattcttaaacagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgccacactgaccaacgcagctggcaagcgggatggcaaggccccagcaggtggcaatggcaacaagaagaagtcgggcaagaaggagaagaagtaacatggaggccaggccaagagccacagggcggc

SEQ ID NO: 28 - protein, 929 aa, Homo sapiens
MGGSCAQRRRAGPRQVLFPLLLPLFYPTLCEPIRYSIPEELAKGSVVGNLAKDLGLSVLDVSARELRVSAEKLHFSVDAQSGDLLVKDRIDREQICKERRRCELQLEAVVENPLNIFHVIVVIEDVNDHAPQFRKDEINLEISESVSLGMGTILESAEDPDISMNSLSKYQLSPNEYFSLVEKDNPDGGKYPELVLQKTLDRETQSAHHLILTALDGGDPPRSATAHIEISVKDTNDNPPVFSRDEYRISLSENLPPGSPVLQVTATDQDEGVNAEINYYFRSTAQSTKHMFSLDEKTGMIKNNQSFDFEDVERYTMEVEAKDGGGLSTQCKVIIEILDENDNSPEIIITSLSDQILENSPPGMVVALFKTRDLDFGGNGEVRCNIETDIPFKIYSSSNNYYKLVTDGALDREQTPEYNVTIVATDRGKPPLSSSRSITLYVADINDNAPVFDQTSYVVHVAENNPPGASIAQVSASDPDLGLNGHISYSLIASDLESRTLSSYVSVSAQSGVVFAQRAFDHEQLRAFALTLQARDQGSPALSANVSLRVLVDDRNDNAPRVLYPALGPDGSAFFDMVPRSAEPGYLVTKVVAVDADSGHNAWLSYHVLQASEPGLFSLGLRTGEVRMVRALGDKDSVRQRLLVAIRDGGQPPLSATATLHLVFADSLQEVLPDFSDHPTPSDSQAEMQFYLVVALALISVLFLLAVILAIALRLRQSFSPTAGDCFESVLCSKSGPVGPPNYSEGTLPYAYNFCVPGDQMNPEFNFFTSVDHCPATQDNLNKDSMLLASILTPSVEADKKILKQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 29 - DNA, 2436 bp, Homo sapiens
  CDS (1)..(2397)
atggctgtcagagagttgtgcttcccaagacaaaggcaagtcctgtttctttttcttttttggggagtgtccttggcaggttctgggtttggacgttattcggtgactgaggaaacagagaaaggatcctttgtggtcaatctggcaaaggatctgggactagcagagggggagctggctgcaaggggaaccagggtggtttccgatgataacaaacaatacctgctcctggattcacataccgggaatttgctcacaaatgagaaactggaccgagagaagctgtgtggccctaaagagccctgtatgctgtatttccaaattttaatggatgatccctttcagatttaccgggctgagctgagagtcagggatataaatgatcacgcgccagtatttcaggacaaagaaacagtcttaaaaatatcagaaaatacagctgaagggacagcatttagactagaaagagcacaggatccagatggaggacttaacggtatccaaaactacacgatcagccccaactcttttttccatattaacattagtggcggtgatgaaggcatgatatatccagagctagtgttggacaaagcactggatcgggaggagcagggagagctcagcttaaccctcacagcgctggatggtgggtctccatccaggtctgggacctctactgtacgcatcgttgtcttggacgtcaatgacaatgccccacagtttgcccaggctctgtatgagacccaggctccagaaaacagccccattgggttccttattgttaaggtatgggcagaagatgtagactctggagtcaacgcggaagtatcctattcattttttgatgcctcagaaaatattcgaacaacctttcaaatcaatcctttttctggggaaatctttctcagagaattgcttgattatgagttagtaaattcttacaaaataaatatacaggcaatggacggtggaggcctttctgcaagatgtagggttttagtggaagtattggacaccaatgacaatccccctgaactgatcgtatcatcattttccaactctgttgctgagaattctcctgagacgccgctggctgtttttaagattaatgacagagactctggagaaaatggaaggatggtgtgctacattcaagatgatctgccattcctactaaaaccttctgttgagaatttttacatcctaatgactgaaggcgcgctggacagagaagcaagagctgaatataatatcaccctcaccgtcacagatatggggactccaaggctgaaaacggagcacaacataacagtgcagatatcagatgtcaatgataacgcccccactttcacccaaacctcctacgccctgttcgtccgcgagaacaacagccccgccctgcacatcggcagcgtcagcgccacagacagagactcaggcaccaacgcccaggtcacctactcgctgctgccgccccaggacccgcacctgcccctcgcctccctggtctccatcaacgcagacaacggccacctgttcgccctcaggtcgctggactacgaggccctgcaggctttcgagttccgcgtgggcgccacagaccgcggctcccccgcgctgagcagagaggcgctggtgcgcgtgctggtgctggacgccaacgacaactcgcccttcgtgctgtacccgctgcagaacggctccgcgccctgcactgagctggtgccccgggcggccgagccgggctacctggtgaccaaggtggtggcggtggacggcgactcgggccagaacgcctggctgtcgtaccagctgctcaaggccacggagcccgggctgttcggtgtgtgggcgcacaatggggaggtgcgcaccgccaggctgctgagcgagcgcgacgcagccaagcacaggctcgtggtgcttgtcaaggacaatggcgagcctcctcgctcggccaccgccacgctgcacttgctcctggtggacggcttctcccagccctacctgcctctcccggaggcggccccggcccaggcccaggccgaggccgacttgctcaccgtctacctggtggtggcgttggcctcggtgtcttcgctcttcctcctctcggtgctcctgttcgtggcggtgcggctgtgcaggaggagcagggcggcctcggtgggtcgctgctcggtgcccgagggtccttttccagggcatctggtggacgtgagcggcaccgggaccctgttccagagctaccagtacgaggtgtgtctgactggaggttcagagaccggcgagttcaagttcttgaagccgattaccccccacctcccgccccataggggtgggaaagaaatagaggaaaattctactctccccaatagctttggatttaattattgaaaggaacccacttaataaagacatttacttctttaa

SEQ ID NO: 30 - protein, 799 aa, Homo sapiens
MAVRELCFPRQRQVLFLFLFWGVSLAGSGFGRYSVTEETEKGSFVVNLAKDLGLAEGELAARGTRVVSDDNKQYLLLDSHTGNLLTNEKLDREKLCGPKEPCMLYFQILMDDPFQIYRAELRVRDINDHAPVFQDKETVLKISENTAEGTAFRLERAQDPDGGLNGIQNYTISPNSFFHINISGGDEGMIYPELVLDKALDREEQGELSLTLTALDGGSPSRSGTSTVRIVVLDVNDNAPQFAQALYETQAPENSPIGFLIVKVWAEDVDSGVNAEVSYSFFDASENIRTTFQINPFSGEIFLRELLDYELVNSYKINIQAMDGGGLSARCRVLVEVLDTNDNPPELIVSSFSNSVAENSPETPLAVFKINDRDSGENGRMVCYIQDDLPFLLKPSVENFYILMTEGALDREARAEYNITLTVTDMGTPRLKTEHNITVQISDVNDNAPTFTQTSYALFVRENNSPALHIGSVSATDRDSGTNAQVTYSLLPPQDPHLPLASLVSINADNGHLFALRSLDYEALQAFEFRVGATDRGSPALSREALVRVLVLDANDNSPFVLYPLQNGSAPCTELVPRAAEPGYLVTKVVAVDGDSGQNAWLSYQLLKATEPGLFGVWAHNGEVRTARLLSERDAAKHRLVVLVKDNGEPPRSATATLHLLLVDGFSQPYLPLPEAAPAQAQAEADLLTVYLVVALASVSSLFLLSVLLFVAVRLCRRSRAASVGRCSVPEGPFPGHLVDVSGTGTLFQSYQYEVCLTGGSETGEFKFLKPITPHLPPHRGGKEIEENSTLPNSFGFNY

SEQ ID NO: 31 - DNA, 1327 bp, Homo sapiens
  CDS (67)..(1272)
gcttcaccactccctccaccttctccaccaactaccagtccctgggctctgtccagccgcccagctatggcacctggccggtcagcagcgcagccagcatctatgcaggcactggggggcttgggatcccagatctccatgtcctgttctaccagtttctggggcggcttggggtctgggggcctggccacagagatggctgggggtctggcagaaatggggggcatccagaatgagaaggagaccatgcaaagcctgaacgaccacctggactacctggacagagtgaggaacctggagaccgagaactggaggctggagagcaaaatccaggagtatctggagaagagaccccatgtcagagactggggccattacttcaagaccatcaaggaactgagggctcagatcttcgcaaatactgtggacaatgtccacatcattctgcagatcgacaatgcccgtcttgctgctgatgacttcagagtcaagtatgagacaagagctggccatgcgccagtctgtggagagaacatccatgggctctgcaaggtcattgatgacaccaatgtcactctgctgcagctggagacagagatgggcgctctcaaggaggagctgctcctcatgaagaagaaccatgaagaggaagtaaaaggcttgcaagtccagattgccaactctgggttggccgtggaggtagatgcccccaaatctcaagtcctcgccaaggtcatggcagacatcagggcccaagatgagctgtctcagaagaactcagagaagctaggcaagtactggtctcagcagactgaggagagcaccacagtggtcaccacacactctgccaaggtcagagctgctgagatgacaacggagctgagacgtacagtccagtgcttggagattgacctggactcaatgagaaatctgaagaccagcttgaacagcctgagggaggtggaggcccgctacgccctgcagatggagcagctcaacagaatcctgctgtacttggagtcaaagctggcacagaactgggcagagggccagcgcaaggtccaggagtacaaggacttgctgaacatcagggtcaagctggaggctgagatcgccacctaccgccgcctgctggaagacagcgagggcctcaatcttggtgatgccctggacagcagcaactccatgcaaaccatccaaaagaccaccacccgccagatagtggatagcaaagtggtgtctgagatcagtgacaccaaagttctgagacattaagccagcagaagcagggtaccctgtggggagtaagaggccaataaaaagttca

SEQ ID NO: 32 - protein, 402 aa, Homo sapiens
MAPGRSAAQPASMQALGGLGSQISMSCSTSFWGGLGSGGLATEMAGGLAEMGGIQNEKETMQSLNDHLDYLDRVRNLETENWRLESKIQEYLEKRPHVRDWGHYFKTIKELRAQIFANTVDNVHIILQIDNARLAADDFRVKYETRAGHAPVCGENIHGLCKVIDDTNVTLLQLETEMGALKEELLLMKKNHEEEVKGLQVQIANSGLAVEVDAPKSQVLAKVMADIRAQDELSQKNSEKLGKYWSQQTEESTTVVTTHSAKVRAAEMTTELRRTVQCLEIDLDSMRNLKTSLNSLREVEARYALQMEQLNRILLYLESKLAQNWAEGQRKVQEYKDLLNIRVKLEAEIATYRRLLEDSEGLNLGDALDSSNSMQTIQKTTTRQIVDSKVVSEISDTKVLRH

SEQ ID NO: 33 - DNA, 22 bp, Homo sapiens
gatggtacgtgatgctcttgtt

SEQ ID NO: 34 - DNA, 26 bp, Homo sapiens
cccttgccgataggaagttctttgct

SEQ ID NO: 35 - DNA, 22 bp, Homo sapiens
aggaagctgcacatttgttcta

SEQ ID NO: 36 - DNA, 22 bp, Homo sapiens
gggtgtttaggattccctgtag

SEQ ID NO: 37 - DNA, 26 bp, Homo sapiens
ccaattgaaagagtctccaggcatca

SEQ ID NO: 38 - DNA, 22 bp, Homo sapiens
tgcagttgattcagaacatgag

SEQ ID NO: 39 - DNA, 20 bp, Homo sapiens
actgggaacaggcactgtct

SEQ ID NO: 40 - DNA, 23 bp, Homo sapiens
cagcagaactgtccagcccatgg

SEQ ID NO: 41 - DNA, 22 bp, Homo sapiens
gggtctgtcagagcatcagtac

SEQ ID NO: 42 - DNA, 19 bp, Homo sapiens
cagtctccaggccatgaag

SEQ ID NO: 43 - DNA, 23 bp, Homo sapiens
accttgtccctggtcctgctggt

SEQ ID NO: 44 - DNA, 20 bp, Homo sapiens
agaccctgagctctctccat

SEQ ID NO: 45 - DNA, 22 bp, Homo sapiens
gtgttgctaggaagcatgttct

SEQ ID NO: 46 - DNA, 25 bp, Homo sapiens
acacgtgcagtagctgcacctgctt

SEQ ID NO: 47 - DNA, 21 bp, Homo sapiens
atgacacatgcgacgtatgag

SEQ ID NO: 48 - DNA, 22 bp, Homo sapiens
gaggtacccactgtggtacaag

SEQ ID NO: 49 - DNA, 26 bp, Homo sapiens
acatggtgtccctgtggatcctcttt

SEQ ID NO: 50 - DNA, 22 bp, Homo sapiens
gggagaggatgagtttgatgat

SEQ ID NO: 51 - DNA, 22 bp, Homo sapiens
ggctccttcttcttttctgtgt

SEQ ID NO: 52 - DNA, 26 bp, Homo sapiens
atcaccaccattggctatggcaacct

SEQ ID NO: 53 - DNA, 21 bp, Homo sapiens
ggcaaagaagatgcagaagag

SEQ ID NO: 54 - DNA, 21 bp, Homo sapiens
gctatgcaggtggtctgagtt

SEQ ID NO: 55 - DNA, 24 bp, Homo sapiens
ctcacaagccctggcctcagctat

SEQ ID NO: 56 - DNA, 20 bp, Homo sapiens
agagccaaagctggagctta

SEQ ID NO: 57 - DNA, 22 bp, Homo sapiens
cttgtgctagtgatcgacatca

SEQ ID NO: 58 - DNA, 27 bp, Homo sapiens
ccccttattctcaatcctcctactacg

SEQ ID NO: 59 - DNA, 22 bp, Homo sapiens
ccgttgttttcgtttacgtaaa

SEQ ID NO: 60 - DNA, 22 bp, Homo sapiens
ggagactgctttgagtcagttc

SEQ ID NO: 61 - DNA, 23 bp, Homo sapiens
tctgctccaagtccggacctgtg

SEQ ID NO: 62 - DNA, 22 bp, Homo sapiens
aattataggcatagggcaacgt

SEQ ID NO: 63 - DNA, 21 bp, Homo sapiens
gactgaggagagcaccacagt

SEQ ID NO: 64 - DNA, 25 bp, Homo sapiens
acactctgccaaggtcagagctgct

SEQ ID NO: 65 - DNA, 21 bp, Homo sapiens
gtctcagctccgttgtcatct

SEQ ID NO: 66 - protein, 248 aa, Homo sapiens
TGKVALVTGASSGIGLAIAKRLAEEGAKVVVVDRREEKAEAAAELKAELGDRALFIQLDVTDEESIKAAVAQAVEELGRLDVLVNNAGILGPGEPFELSEDDWERVIDVNLTGVFLLTQAVLPHMLKRSGGRIVNISSVAGLVPSPGLSAYSASKAAVVGFTRSLALELAPHGIRVNAIAPGGVDTDMTKALRSIAPADPELVERITSALVPLGRYGTPEEVANAVLFLASDGASYSVTGQTLNVDGG

SEQ ID NO: 67 - protein, 148 aa, Homo sapiens
RVALEHDPPADTSAVGLWVDAGSRYEPDDNNGLAHFLEHMAFKGTKKYPSNELEEELEKLGGSLNAYTSREHTAYYVEVLNDDLPKAVDRLADFFLNPLFSPSEVERERLVVLYEVEAVDAEPQAVLLDNLHAAAYRGTPLGRSLLGP

SEQ ID NO: 68 - protein, 175 aa, Homo sapiens
LDVVFLLDGSGSMGGNRFELAKEFVLKLVEQLDIGPDGDRVGLVTFSSDARVLFPLNDSQSKDALLEALASLSYSLGGGTNLGAALEYALENLFSESAGSRRGAPKVLILITDGESNDGGEDILKAAKELKRSGVKVFVVGVGNDVDEEELKKLASAPGGVFVVEDLPSLLDLLI

SEQ ID NO: 69 - protein, 315 aa, Homo sapiens
AFKQLGWIPGLVLLLLAGFITLYTGLLLSECYEYVPGKRNDSYLDLGRSAYGGKGLLLTSFVGQYVNLFGVNIGYLILAGDLLPKIISSFCGDNCDHLDGNSWIIIFAAIIITLSFIPNFNLLSISSLSAFSSLAYLSIISFLIIVAVIAGIFVLLGAVYGILWSPSFTKLTGLFLAIGIIVFAFEGHAVLLPIQNTMKSPSAKKFKKVLNVAIIIVTVLYILVGFFGYLTFGNNVKGNILLNLPNNPFWLIVNLNLVVAILLTFPLQAFPIVRIIENLLTKKNNFAPNKSKLLRVVIRSGLVVFTLLIAILVPF

SEQ ID NO: 70 - protein, 311 aa, Homo sapiens
NEKEQMQNLNDRLASYIDKVRFLEQQNKELEVKIEELRQKQAPSVSRLYSLYETEIEELRRQIDQLTNERARLQLEIDNLREAAEDFRKKYEDEINLRQEAENDLVGLRKDLDEATLARVDLENKVESLQEELEFLKKNHEEEVKELQAQIQDTVNVEMDAARKLDLTKALREIRAQYEEIAKKNRQEAEEWYKSKLEELQTAAARNGEALRSAKEEITELRRQIQSLEIELQSLKAQNASLERQLAELEERYELELRQYQALISQLEEELQQLREEMARQLREYQELLDVKLALDIEIATYRKLLEGEES

SEQ ID NO: 71 - protein, 310 aa, Homo sapiens
NEKEQMQNLNDRLASYIDKVRFLEQQNKELEVKIEELRQKQAPSVSRLYSLYETEIEELRRQIDQLTNERARLQLEIDNLREAAEDFRKKYEDEINLRQEAENDLVGLRKDLDEATLARVDLENKVESLQEELEFLKKNHEEEVKELQAQIQDTVNVEMDAARKLDLTKALREIRAQYEEIAKKNRQEAEEWYKSKLEELQTAAARNGEALRSAKEEITELRRQIQSLEIELQSLKAQNASLERQLAELEERYELELRQYQALISQLEEELQQLREEMARQLREYQELLDVKLALDIEIATYRKLLEGEE